(19) United States
(12) Patent Application Publication
(10) Pub. No.: US 2025/0161607 A1
(43) Pub. Date: May 22, 2025

(54) SYSTEM AND METHOD FOR DETERMINING LEAK IN RESPIRATORY FLOW THERAPY SYSTEMS

(71) Applicant: Fisher & Paykel Healthcare Limited, Auckland (NZ)

(72) Inventors: Lucy Elizabeth ELLINGHAM, Auckland (NZ); Samuel Carey Mathew SANSON, Auckland (NZ)

(21) Appl. No.: 18/563,080

(22) PCT Filed: May 25, 2022
(86) PCT No.: PCT/IB2022/054869

§ 371 Date: Nov. 21, 2023

Related U.S. Application Data
(60) Provisional application No. 63/192,964, filed on May 25, 2021.

(51) Int. Cl. A61M 16/00; A61M 16/10; G16H 40/63

(52) U.S. Cl. CPC A61M 16/024; A61M 16/0003; A61M 16/0051; A61M 16/109; G16H 40/63; A61M 2016/0027; A61M 2202/0007; A61M 2202/02; A61M 2205/10; A61M 2205/15; A61M 2205/18; A61M 2205/3327; A61M 2205/3334; A61M 2205/3365; A61M 2205/3368; A61M 2205/36; A61M 2205/502; A61M 2205/583

(57) ABSTRACT

A respiratory apparatus for providing a flow of gases to a user for respiratory therapy. The respiratory apparatus comprises a controller that is operable to execute one or more leak detection algorithms to determine leaks and/or components missing in the flow path. The leak detection algorithms compare pressure and/or flow rate signals to one or more thresholds using a two-stage evaluation process to determine leaks.

### FIELD OF THE DISCLOSURE

The present disclosure relates to methods and systems for providing a respiratory flow therapy to a patient. In particular, the present disclosure relates to determining leaks in the flow path of a respiratory apparatus that delivers a flow of gases to a patient via an unsealed interface.

### BACKGROUND

Breathing assistance apparatuses are used in various environments such as hospital, medical facility, residential care, or home environments to deliver a flow of gases to users or patients. A breathing assistance apparatus or respiratory therapy apparatus (collectively, ‘respiratory apparatus’ or ‘respiratory device’) may be used to deliver supplementary oxygen or other gases with a flow of gases, and/or a humidification apparatus to deliver heated and humidified gases. A respiratory apparatus may allow adjustment and control over characteristics of the gases flow, including flow rate, temperature, gases concentration, humidity, pressure, etc. Sensors, such as flow sensors and/or pressure sensors are used to measure characteristics of the gases flow.

### SUMMARY

The present disclosure provides methods and processes for detecting a leak or leaks in the flow path of a respiratory apparatus. In one configuration, the respiratory apparatus is an ‘unsealed system’ that provides high flow therapy. The high flow therapy may be a nasal high flow system which typically delivers a flow of gases via an unsealed interface, such as a nasal cannula, or a tracheal high flow system that delivers a flow of gases via a tracheostomy interface. In some configurations, the leak detection algorithm may operate while the patient is connected or partially connected (i.e. with interface on or partially fitted) and receiving therapy, and/or when the patient is disconnected from the system (i.e. with interface off). In an unsealed interface system, such as a nasal high flow system, leaks in the flow path of the apparatus are difficult to determine because of the open and unsealed nature of the system.

The processes disclosed herein can be used when the patient interface is a non-sealed device, such as a nasal cannula in a nasal high flow therapy, or via an unsealed tracheal interface to provide tracheal high flow therapy. Nasal high flow and tracheal high flow are collectively known as “high flow therapy”.

In an aspect, the present disclosure relates to a respiratory apparatus that is configured to provide a flow of gases to a user for respiratory therapy comprising:
a motor-driven flow generator that is operable to generate a flow of gases;
a flow path for the flow of gases through the respiratory apparatus to a gases outlet of the respiratory apparatus;
a humidifier that is operable to heat and humidify the flow of gases, the humidifier comprising a removable humidification chamber in the flow path;
a pressure sensor configured to generate a pressure variable representing a sensed pressure characteristic of the flow of gases in the flow path; and
a controller that is operatively connected to the flow generator and operable to control a flow rate of the flow of gases by controlling a motor speed of the flow generator, wherein the controller is configured during operation to execute a leak detection process for detecting leaks in the flow path by:
   initiating a first-stage leak evaluation comprising comparing the pressure variable to a first leak threshold, the first leak threshold representing a possible leak condition; and
   initiating a second-stage leak evaluation if the pressure variable is below the first leak threshold thereby indicating a possible leak, the second-stage leak evaluation comprising:
   increasing the motor speed of the flow generator from the current motor speed to a higher motor speed,
   comparing a new pressure variable at the higher motor speed to the first leak threshold and a second leak threshold, the second leak threshold representing a definite leak condition, and then:
   generating a leak alarm and exiting the second-stage leak evaluation if the new pressure variable is below the second leak threshold thereby confirming the possible leak as a definite leak; or
   exiting the second-stage leak evaluation without generating an alarm if the new pressure variable is above the first leak threshold and second leak threshold thereby resolving the possible leak as no leak; or
   repeating the second-stage leak evaluation at a further higher motor speed or speeds until the possible leak is confirmed as a leak or resolved as no leak.

In a configuration, the first-stage leak evaluation comprises comparing the pressure variable to the first leak threshold and second leak threshold, and wherein the controller is configured to initiate the second-stage leak evaluation if the pressure variable is below the first leak threshold and above the second leak threshold thereby indicating a possible leak.

In a configuration, the first-stage leak evaluation comprises comparing the pressure variable to the second leak threshold, and wherein the controller is configured to generate a leak alarm if the pressure variable is below the second leak threshold.

In a configuration, the controller is configured to repeat or continue in the first-stage leak evaluation during normal operation until an exit condition occurs.

In a configuration, the exit conditions comprise generating a leak alarm or initiating a second-stage leak evaluation to resolve a possible leak as a definite leak or no leak.

In a configuration, in the second-stage leak evaluation, increasing the motor speed of the flow generator comprises increasing the motor speed by a set increment to a higher motor speed or to the next higher motor speed from a predetermined series or array of higher motor speeds.

In a configuration, in the second-stage leak evaluation, the controller is configured to hold the flow generator at the higher motor speed for a predetermined time period while comparing the new pressure variable to the first and/or second leak thresholds to resolve the possible leak as a definite leak or no leak.

In a configuration, the controller is configured to repeat the second-stage leak evaluation at a next further higher motor speed if the possible leak is not confirmed as a leak or resolved as no leak at the present higher motor speed within a predetermined time period.

In a configuration, the controller is configured to exit the second-stage leak evaluation depending on a comparison of the current motor speed to a motor speed threshold.

In a configuration, the controller is configured to exit the second-stage leak evaluation if the current higher motor speed operating during the second-stage leak evaluation not below a motor speed threshold.

In a configuration, the controller is configured to adjust the first leak threshold if the second-stage leak evaluation is exited without generating an alarm.

In a configuration, the controller is configured to adjust the first leak threshold by reducing the first leak threshold associated with the flow rate and/or motor speed operating during the first-stage leak evaluation.

In a configuration, the values of the first and second leak thresholds are at least partly dependent on or a function of the flow rate and/or motor speed operating at the time of the comparison.

In a configuration, the first and second leak thresholds are extracted from respective pressure-flow characteristic curves and/or representative look-up tables designating the thresholds for a range of flow rates and/or motor speeds.

In a configuration, the controller is configured to determine each comparison evaluation of the pressure variable to the first and/or second leak thresholds in the leak evaluation stages based on whether the pressure variable is consistently above or below the threshold for a respective minimum evaluation time period.

In a configuration, each minimum evaluation time period is dependent on the specific comparison evaluation and/or specific leak evaluation stage.

In a configuration, the minimum evaluation time period(s) associated with comparison evaluations in the second-stage leak evaluation are shorter than the minimum evaluation time period(s) associated with comparison evaluations in the prior first-stage leak evaluation.

In a configuration, the controller is configured to initiate one or more alarm actions when generating a leak alarm.

In a configuration, the alarm action comprises freezing the flow rate and/or motor speed of the flow generator to the current operating settings.

In a configuration, the alarm action comprises generating a notification or indication of the leak on a display of the apparatus.

In a configuration, the leak alarm indicates that the removable humidification chamber has been at least partially or entirely removed or disconnected from the flow path of the apparatus.

In a configuration, the leak alarm indicates that a patient circuit has been at least partially or entirely removed or disconnected from the gases outlet of the apparatus.

In a configuration, the controller is operable to detect one or more different types of leaks in the flow path of the apparatus, each different type of leak having its own respective first and second leak thresholds.

In a configuration, after alarm is generated, the controller is configured to maintain the alarm until the pressure variable rises above the second leak threshold or an alternative resolution leak threshold for a minimum time period.

In a configuration, after an alarm is generated, the controller is configured to disable the alarm and revert to normal operation if the pressure variable rises above the second leak threshold or an alternative resolution leak threshold for a minimum time period.

In a configuration, the controller is configured to operate the leak detection process continuously without modification across an entire or substantial portion of the operational flow rate range of the apparatus. In one example, the leak detection process is operable across an entire operational flow rate range of the apparatus, which may be 0 L/min to 90 L/min, and with a range of different types and/or sizes of patient interfaces.

In a configuration, the respiratory apparatus comprises a patient circuit, including a patient interface, connected to the gases outlet and wherein the controller is configured to operate the leak detection process continuously without modification for a range of different types or sizes of patient interface.

In a configuration, the humidifier of the respiratory apparatus further comprises a heater plate that is operable to heat the humidification chamber, and wherein the controller is further configured to execute a heater plate check process to further confirm or validate a definite leak identified in the second-stage leak evaluation before generating the leak alarm.

In a configuration, the heater plate check process comprises applying a power or temperature process to the heater plate and evaluating the heating rate or and/or cooling rate of the heater plate against one or more thresholds based on a temperature sensor of or associated with the heater plate to thereby determine the presence or absence of a humidification chamber being in thermal contact with the heater plate, the absence of the humidification chamber validating a definite leak condition.

In another aspect, the present disclosure relates to a method of detecting a leak in the flow path of a respiratory apparatus that is configured to provide a flow of gases to a user at a controllable flow rate for respiratory therapy, comprising:
receiving a pressure variable from a pressure sensor of the apparatus, the pressure variable representing a sensed pressure characteristic of the flow of gases in the flow path;
initiating a first-stage leak evaluation comprising comparing the pressure variable to a first leak threshold, the first leak threshold representing a possible leak condition; and
initiating a second-stage leak evaluation if the pressure variable is below the first leak threshold thereby indicating a possible leak, the second-stage leak evaluation comprising:
   increasing the motor speed of the flow generator from the current motor speed to a higher motor speed,
   comparing a new pressure variable at the higher motor speed to the first leak threshold and a second leak threshold, the second leak threshold representing a definite leak condition, and then:
   generating a leak alarm and exiting the second-stage leak evaluation if the new pressure variable is below the second leak threshold thereby confirming the possible leak as a definite leak; or
   exiting the second-stage leak evaluation without generating an alarm if the new pressure variable is above the first leak threshold and second leak threshold thereby resolving the possible leak as no leak; or
   repeating the second-stage leak evaluation at a further higher motor speed or speeds until the possible leak is confirmed as a leak or resolved as no leak.

In a configuration, the first-stage leak evaluation comprises comparing the pressure variable to the first leak threshold and second leak threshold, and wherein the method comprises initiating the second-stage leak evaluation if the pressure variable is below the first leak threshold and above the second leak threshold thereby indicating a possible leak.

In a configuration, the first-stage leak evaluation comprises comparing the pressure variable to the second leak threshold, and generating a leak alarm if the pressure variable is below the second leak threshold.

In a configuration, the method comprises repeating the first-stage leak evaluation during normal operation until an exit condition occurs.

In a configuration, the exit conditions comprise generating a leak alarm or initiating a second-stage leak evaluation to resolve a possible leak as a definite leak or no leak.

In a configuration, in the second-stage leak evaluation, increasing the motor speed of the flow generator comprises increasing the motor speed by a set increment to a higher motor speed or to the next higher motor speed from a predetermined series or array of higher motor speeds.

In a configuration, in the second-stage leak evaluation, the method comprises holding the flow generator at the higher motor speed for a predetermined time period while comparing the new pressure variable to the first and/or second leak thresholds to resolve the possible leak as a definite leak or no leak.

In a configuration, the method comprises repeating the second-stage leak evaluation at a next further higher motor speed if the possible leak is not confirmed as a leak or resolved as no leak at the present higher motor speed within a predetermined time period.

In a configuration, the method comprises exiting the second-stage leak evaluation depending on a comparison of the current motor speed to a motor speed threshold.

In a configuration, the method comprises exiting the second-stage leak evaluation if the current higher motor speed operating during the second-stage leak evaluation is not below a motor speed threshold.

In a configuration, the method comprises adjusting the first leak threshold if the second-stage leak evaluation is exited without generating an alarm.

In a configuration, the method comprises adjusting the first leak threshold by reducing the first leak threshold associated with the flow rate and/or motor speed operating during the first-stage leak evaluation.

In a configuration, the values of the first and second leak thresholds are at least partly dependent on or a function of the flow rate and/or motor speed operating at the time of the comparison.

In a configuration, the method comprises extracting the first and second leak thresholds from respective pressure-flow characteristic curves and/or representative look-up tables designating the thresholds for a range of flow rates and/or motor speeds.

In a configuration, the method comprises determining each comparison evaluation of the pressure variable to the first and/or second leak thresholds in the leak evaluation stages based on whether the pressure variable is consistently above or below the threshold for a respective minimum evaluation time period.

In a configuration, the minimum evaluation time period is dependent on the specific comparison evaluation and/or specific leak evaluation stage.

In a configuration, the minimum evaluation time period(s) associated with comparison evaluations in the second-stage leak evaluation are shorter than the minimum evaluation time period(s) associated with comparison evaluations in the prior first-stage leak evaluation.

In a configuration, the method comprises initiating one or more alarm actions when generating a leak alarm.

In a configuration, the alarm action comprises freezing the flow rate and/or motor speed of the flow generator to the current operating settings.

In a configuration, the alarm action comprises generating a notification or indication of the leak on a display of the apparatus.

In a configuration, the leak alarm indicates that the removable humidification chamber has been at least partially or entirely removed or disconnected from the flow path of the apparatus.

In a configuration, the leak alarm indicates that a patient circuit has been at least partially or entirely removed or disconnected from the gases outlet of the apparatus.

In a configuration, the method comprises detecting one or more different types of leaks in the flow path of the apparatus, each different type of leak having its own respective first and second leak thresholds.

In a configuration, after alarm is generated, the method comprises maintaining the alarm until the pressure variable rises above the second leak threshold or an alternative resolution leak threshold for a minimum time period.

In a configuration, after an alarm is generated, the method comprises disabling the alarm and reverting to normal operation if the pressure variable rises above the second leak threshold or an alternative resolution leak threshold for a minimum time period.

In a configuration, the apparatus comprises:
a motor-driven flow generator that is operable to generate a flow of gases;
a flow path for the flow of gases through the respiratory apparatus to a gases outlet of the respiratory apparatus;
a humidifier that is operable to heat and humidify the flow of gases, the humidifier comprising a removable humidification chamber in the flow path;
a pressure sensor configured to generate a pressure variable representing a sensed pressure characteristic of the flow of gases in the flow path; and
a controller that is operatively connected to the flow generator and operable to control a flow rate of the flow of gases by controlling a motor speed of the flow generator, wherein the method is executable or implemented by the controller.

In a configuration, the method operates continuously without modification across an entire or substantial portion of the operational flow rate range of the apparatus. In some embodiments, the flow rate range may be 0 L/min to 90 L/min, for example.

In a configuration, the respiratory apparatus comprises a patient circuit, including a patient interface, connected to the gases outlet and wherein the method operates continuously without modification for a range of different types or sizes of patient interface.

In another aspect, the present disclosure relates to a respiratory apparatus that is configured to provide a flow of gases to a user for respiratory therapy comprising: a motor-driven flow generator that is operable to generate a flow of gases;
a flow path for the flow of gases through the respiratory apparatus to a gases outlet of the respiratory apparatus;
a humidifier that is operable to heat and humidify the flow of gases, the humidifier comprising a removable humidification chamber in the flow path;
a pressure sensor configured to generate a pressure variable representing a sensed pressure characteristic of the flow of gases in the flow path; and
a controller that is operatively connected to the flow generator and operable to control a flow rate of the flow of gases by controlling a motor speed of the flow generator, wherein the controller is configured during operation to:
   receive the pressure variable from the pressure sensor at a first flow rate;
   determine if a possible leak condition is satisfied based on comparing the received pressure variable to a first threshold, the first threshold representing a possible leak condition, and
if the possible leak condition is satisfied:
   increase the motor speed of the flow generator to generate a second flow rate for the flow of gases;
   receive an updated pressure variable at the second flow rate;
   determine which of a possible leak condition, no leak condition, or definite leak condition is satisfied by comparing the updated pressure variable to the first threshold and/or a second threshold, the second threshold representing a definite leak condition, and
if the possible leak condition is again satisfied at the second flow rate:
   continually increase the motor speed of the flow generator by discrete increments to increase the flow rate of the gases in discrete intervals and re-comparing the updated pressure variable to the first and/or second thresholds after each discrete increment until either a definite leak condition or no leak condition is satisfied.

In a configuration, the possible leak condition is satisfied if the pressure variable is below the first threshold.

In a configuration, the possible leak condition is satisfied if the pressure variable is below the first threshold and above the second threshold.

In a configuration, the no leak condition is satisfied if the pressure variable is above the first and second thresholds.

In a configuration, the definite leak condition is satisfied if pressure variable is below the second threshold.

In a configuration, the controller is configured to determine if a condition is satisfied based on whether the pressure variable is above or below the relevant first and/or second thresholds for a minimum evaluation time period.

In a configuration, the minimum evaluation time period is dependent on the specific condition being evaluated.

In a configuration, the humidifier of the respiratory apparatus further comprises a heater plate that is operable to heat the humidification chamber, and wherein the controller is further configured to execute a heater plate check process to further confirm or validate a definite leak identified in the second-stage leak evaluation before generating the leak alarm.

In a configuration, the heater plate check process comprises applying a power or temperature process to the heater plate and evaluating the heating rate or and/or cooling rate of the heater plate against one or more thresholds based on a temperature sensor of or associated with the heater plate to thereby determine the presence or absence of a humidification chamber being in thermal contact with the heater plate, the absence of the humidification chamber validating a definite leak condition.

In an aspect, the present disclosure relates to a method of detecting a leak in the flow path of a respiratory apparatus that is configured to provide a flow of gases to a user at a controllable flow rate for respiratory therapy, comprising:
receiving a pressure variable from a pressure sensor of the apparatus operating at a first flow rate, the pressure variable representing a sensed pressure characteristic of the flow of gases in the flow path;
determining if a possible leak condition is satisfied based on comparing the received pressure variable to a first threshold, the first threshold representing a possible leak condition, and
if the possible leak condition is satisfied:
   increasing the motor speed of the flow generator to generate a second flow rate for the flow of gases;
   receiving an updated pressure variable at the second flow rate;
   determining which of a possible leak condition, no leak condition, or definite leak condition is satisfied by comparing the updated pressure variable to the first threshold and/or a second threshold, the second threshold representing a definite leak condition, and
if the possible leak condition is again satisfied at the second flow rate:
   continually increasing the motor speed of the flow generator by discrete increments to increase the flow rate of the gases in discrete intervals and re-comparing the updated pressure variable to the first and/or second thresholds until either a definite leak condition or no leak condition is satisfied.

In a configuration, the method comprises determining that the possible leak condition is satisfied if the pressure variable is below the first threshold.

In a configuration, the method comprises determining that the possible leak condition is satisfied if the pressure variable is below the first threshold and above the second threshold.

In a configuration, the method comprises determining that the no leak condition is satisfied if the pressure variable is above the first and second thresholds.

In a configuration, the method comprises determining that the definite leak condition is satisfied if the pressure variable is below the second threshold.

In a configuration, the method comprises determining if a condition is satisfied based on whether the pressure variable is above or below the relevant first and/or second thresholds for a minimum evaluation time period.

In a configuration, the minimum evaluation time period is dependent on the specific condition being evaluated.

In another aspect, the present disclosure relates to a respiratory apparatus that is configured to provide a flow of gases to a user for respiratory therapy comprising:
a motor-driven flow generator that is operable to generate a flow of gases;
a flow path for the flow of gases through the respiratory apparatus to a gases outlet of the respiratory apparatus;
a humidifier that is operable to heat and humidify the flow of gases, the humidifier comprising a removable humidification chamber in the flow path;
a pressure sensor configured to generate a pressure variable representing a sensed pressure characteristic of the flow of gases in the flow path; and
a controller that is operatively connected to the flow generator and operable to control a flow rate of the flow of gases by controlling a motor speed of the flow generator, wherein the controller is configured during operation to:
   control the flow generator or motor speed of the flow generator to deliver the flow of gases at a configurable user set flow rate in normal operation;
   receive the pressure variable from the pressure sensor and either:
      continue normal operation if the pressure variable is above a leak threshold thereby indicating no leak detected; or
      increase the motor speed or user set flow rate of the flow generator by a predetermined or dynamically determined increment if the pressure variable is below the leak threshold indicating a possible leak detected, and receive an updated pressure variable from the pressure sensor at the new increased motor speed or user set flow rate, and either:
      generate an alarm and/or freeze the motor speed if the updated pressure variable is below the leak threshold thereby confirming the possible leak as a leak; or
      revert to normal operation of the flow generator at the original user set flow rate if the pressure variable is above the leak threshold, thereby resolving the possible leak is not a leak.

In another aspect, the present disclosure relates to a method of detecting a leak in the flow path of a respiratory apparatus that is configured to provide a flow of gases to a user at a controllable flow rate for respiratory therapy, comprising:
controlling a flow generator or a motor speed of the flow generator to deliver the flow of gases at a configurable user set flow rate in normal operation;
receiving a pressure variable from a pressure sensor of the apparatus operating at the user set flow rate, the pressure variable representing a sensed pressure characteristic of the flow of gases in the flow path and either:
   continuing normal operation if the pressure variable is above a leak threshold thereby indicating no leak detected; or
   increasing the motor speed or user set flow rate of the flow generator by a predetermined or dynamically determined increment if the pressure variable is below the leak threshold indicating a possible leak detected, and receiving an updated pressure variable from the pressure sensor at the new increased motor speed or user set flow rate, and either:
      generating an alarm and/or freezing the motor speed if the updated pressure variable is below the leak threshold thereby confirming the possible leak as a leak; or
      reverting to normal operation of the flow generator at the original user set flow rate if the pressure variable is above the leak threshold, thereby resolving the possible leak as not a leak.

In another aspect, the present disclosure relates to a respiratory apparatus that is configured to provide a flow of gases to a user for respiratory therapy comprising:
a housing with a receptacle to receive a removable humidification chamber;
a motor-driven flow generator in the housing that is operable to generate a flow of gases;
a gases inlet and gases outlet in the housing;
a flow path for the flow of gases through the respiratory apparatus from the gases inlet through the flow generator and humidification chamber, in use, to the gases outlet, the humidification chamber being in fluid communication with the flow generator and gases outlet when positioned in an operative position in the receptacle of the housing;
a pressure sensor configured to generate a pressure variable representing a sensed pressure characteristic of the flow of gases in the flow path; and
a controller configured to:
   control the flow generator to generate a flow of gases at a configurable user set flow rate;
   receive a pressure variable from the pressure sensor;
   initiate a first-stage leak evaluation comprising comparing the pressure variable to a first leak threshold, the first leak threshold representing a possible leak condition and being a function of the current flow rate and/or motor speed; and
   initiate a second-stage leak evaluation if the pressure variable is below the first leak threshold thereby indicating a possible leak, the second-stage leak evaluation comprising:
   increasing the motor speed of the flow generator from the current motor speed to a higher motor speed,
   comparing a new pressure variable at the higher motor speed to the first leak threshold and a second leak threshold, the second leak threshold representing a definite leak condition and being a function of the current flow rate and/or motor speed, and then:
   generating a leak alarm and exiting the second-stage leak evaluation if the new pressure variable is below the second leak threshold thereby confirming the possible leak as a definite leak; or
   exiting the second-stage leak evaluation if the new pressure variable is above the first leak threshold and second leak threshold thereby resolving the possible leak as no leak; or
   repeating the second-stage leak evaluation at a further higher motor speed or speeds until an alarm is generated or exiting of the second-stage leak evaluation occurs.

In another aspect, the present disclosure relates to a method of determining if a removable humidification chamber has been removed from a flow path of a respiratory apparatus, wherein the respiratory apparatus comprises: a housing with a receptacle to receive the removable humidification chamber; a motor-driven flow generator in the housing that is operable to generate a flow of gases; a gases inlet and gases outlet in the housing; a flow path for the flow of gases through the respiratory apparatus from the gases inlet through the flow generator and humidification chamber, in use, to the gases outlet, the humidification chamber being in fluid communication with the flow generator and gases outlet when positioned in an operative position in the receptacle of the housing; a pressure sensor configured to generate a pressure variable representing a sensed pressure characteristic of the flow of gases in the flow path; and a controller, the method comprising:
controlling the flow generator to generate a flow of gases at a configurable user set flow rate;
receiving a pressure variable from the pressure sensor;
initiating a first-stage leak evaluation comprising comparing the pressure variable to a first leak threshold, the first leak threshold representing a possible leak condition and being a function of the current flow rate and/or motor speed; and
initiating a second-stage leak evaluation if the pressure variable is below the first leak threshold thereby indicating a possible leak, the second-stage leak evaluation comprising:
increasing the motor speed of the flow generator from the current motor speed to a higher motor speed,
comparing a new pressure variable at the higher motor speed to the first leak threshold and a second leak threshold, the second leak threshold representing a definite leak condition and being a function of the current flow rate and/or motor speed, and then:
generating a leak alarm and exiting the second-stage leak evaluation if the new pressure variable is below the second leak threshold thereby confirming the possible leak as a definite leak; or
exiting the second-stage leak evaluation if the new pressure variable is above the first leak threshold and second leak threshold thereby resolving the possible leak as no leak; or
repeating the second-stage leak evaluation at a further higher motor speed or speeds until an alarm is generated or exiting of the second-stage leak evaluation occurs.

In another aspect, the present disclosure relates to a respiratory apparatus that is configured to provide a flow of gases to a user for respiratory therapy comprising:
a motor-driven flow generator that is operable to generate a flow of gases;
a flow path for the flow of gases through the respiratory apparatus to a gases outlet of the respiratory apparatus;
a humidifier that is operable to heat and humidify the flow of gases, the humidifier comprising a removable humidification chamber in the flow path;
a pressure sensor configured to generate a pressure variable representing a sensed pressure characteristic of the flow of gases in the flow path; and
a controller that is operatively connected to the flow generator and operable to control a flow rate of the flow of gases by controlling a motor speed of the flow generator, wherein the controller is configured during operation to:
   control the flow generator or a motor speed of a flow generator to deliver the flow of gases at a configurable user set flow rate;
   if the user set flow rate is below a first flow rate threshold or in a first flow rate range, check for a leak in the flow path by:
   determining if there is a leak based on comparing the pressure variable to one or more leak thresholds, and if a leak or no leak cannot be definitively determined then:
   increasing the user set flow rate or a motor speed of the flow generator by a set increment and re-determining if there is a leak or no leak based on the one or more leak thresholds, and
   if a leak or no leak again cannot be definitively determined, then continually increasing the user set flow rate or motor speed by further set increments, and re-determining if there is a leak or no leak after each increment until either a leak or no leak is definitely determined.

In a configuration, if the user set flow rate is above the first flow rate threshold, the controller is configured to check for a leak in the flow path by:
comparing the pressure variable to a leak threshold at the current user set flow rate and/or motor speed of the flow generator, and determining a leak if the pressure variable ifs below the leak threshold or determining no leak if the pressure variable is above the leak threshold.

In a configuration, the humidifier of the respiratory apparatus further comprises a heater plate that is operable to heat the humidification chamber, and wherein the controller is further configured to execute a heater plate check process to further confirm or validate a definite leak determined.

In a configuration, the heater plate check process comprises applying a power or temperature process to the heater plate and evaluating the heating rate or and/or cooling rate of the heater plate against one or more thresholds based on a temperature sensor of or associated with the heater plate to thereby determine the presence or absence of a humidification chamber being in thermal contact with the heater plate, the absence of the humidification chamber validating the definite leak determined.

In another aspect, the present disclosure relates to a method of determining a leak in the flow path of a respiratory apparatus comprising:
controlling a flow generator or a motor speed of the flow generator to deliver a flow of gases at a configurable user set flow rate;
if the user set flow rate is below a first flow rate threshold or in a first flow rate range, checking for a leak in the flow path by:
   determining if there is a leak based on comparing a pressure variable of the flow of gases to one or more leak thresholds, and if a leak or no leak cannot be definitively determined then:
   increasing the user set flow rate or a motor speed of the flow generator by a set increment and re-determining if there is a leak or no leak based on the one or more leak thresholds, and
   if a leak or no leak again cannot be definitively determined, then continually increasing the user set flow rate or motor speed by further set increments, and re-determining if there is a leak or no leak after each increment until either a leak or no leak is definitely determined.

In a configuration, if the user set flow rate is above the first flow rate threshold, checking for a leak in the flow path by:
comparing the pressure variable to a leak threshold at the current user set flow rate and/or motor speed of the flow generator, and determining a leak if the pressure variable ifs below the leak threshold or determining no leak if the pressure variable is above the leak threshold.

In another aspect, the present disclosure relates to a method of determining a humidification chamber leak condition in a respiratory apparatus, wherein a controller of the respiratory apparatus performs of executes any one of the methods above or below.

In another aspect, the present disclosure relates to a controller of a respiratory apparatus comprising a processor that is configured to perform or execute any one of the methods above or below.

In another aspect, the present disclosure relates to a method of determining a leak in the flow path of a respiratory apparatus according to any one of the methods above, but where the apparatus is operating in a non-therapy mode, such as drying mode and/or disinfection mode.

In another aspect, the present disclosure relates to a method of determining a leak in the flow path of a respiratory apparatus that is operating in a non-therapy mode, comprising:
controlling a flow generator or a motor speed of the flow generator in accordance with the non-therapy mode settings;
determining if there is a leak based on comparing a pressure variable of the flow of gases to one or more leak thresholds, and if a leak or no leak cannot be definitively determined then:
increasing the flow rate or a motor speed of the flow generator by a set increment and re-determining if there is a leak or no leak based on the one or more leak thresholds, and
if a leak or no leak again cannot be definitively determined, then continually increasing the user set flow rate or motor speed by further set increments, and re-determining if there is a leak or no leak after each increment until either a leak or no leak is definitely determined.

In a configuration, the non-therapy mode is a drying mode and/or disinfection mode.

In a configuration, the leak threshold or thresholds are at least partly based on or a function of the flow path characteristics and/or operating settings associated with the drying mode and/or disinfection mode.

In another aspect, the present disclosure relates to respiratory apparatus that is configured to provide a flow of heated and humidified gases to a patient. The apparatus may provide high flow therapy to a patient i.e. user. The apparatus comprising:
a motor driven flow generator that is operable to generate a flow of gases
a flow path for the flow of gases through the respiratory apparatus to a gases outlet of the respiratory apparatus,
a humidifier that is operable to heat and humidify the flow of gases, the humidifier comprising a removable humidification chamber wherein the removable chamber is removable from the flow path such that when the chamber is an operable position the flow path extends through the chamber to the outlet,
a pressure sensor configured to generate a signal indicative of the pressure of the flow of gases,
a flow sensor configured to generate a signal indicative of the flow of the flow of gases,
a motor speed sensor associated with the motor of the flow generator, wherein the motor speed sensor configured to generate a signal indicative of the motor speed of the motor,
a controller operatively coupled to the flow generator to control the flow rate of the flow of gases, the controller in electronic communication with the pressure sensor, flow sensor and motor speed sensor, the controller further configured to:
   perform a first leak detection method using one or more of the pressure sensor, flow sensor and motor speed sensor,
   generate an alarm if the first leak detection method generates a confirmation of a leak,
   perform a second leak detection method if the first leak detection method outputs a possible leak condition, wherein the second leak detection method incrementing the motor speed in predetermined increments until the controller determines a definite leak or definite no leak condition,
   wherein the first leak detection method comprises assessing the measured gases pressure with one of two thresholds for the current measured flow by the flow sensor, and the second leak detection method comparing the measured pressure at a measured flow rate with a leak threshold to confirm a definite leak condition or definite no leak condition, and;
   the controller configured to increment the motor speed by set increments by measuring the motor speed using the motor speed sensor.

In some embodiments, the apparatus is advantageous because it performs a two-stage leak detection to determine a leak in the flow path. The leak in the flow path is a large leak caused by the removal of the humidification chamber and/or conduit coupled to the outlet.

In some embodiments, the apparatus comprises a housing wherein the flow generator and humidifier are located within a common housing. The housing may comprise a receptable to receive the humidification chamber. The controller is configured to perform an action if a definite leak is determined.

In another aspect, the present disclosure relates to a respiratory apparatus that is operable in a disinfection mode to disinfect one or more components in a flow path of the apparatus, comprising:
a motor-driven flow generator that is operable to generate a flow of gases;
a flow path for the flow of gases through the respiratory apparatus;
a removable disinfection kit or assembly forming a part of the flow path;
a pressure sensor configured to generate a pressure variable representing a sensed pressure characteristic of the flow of gases in the flow path; and
a controller that is operatively connected to the flow generator and operable to control a flow rate of the flow of gases by controlling a motor speed of the flow generator, wherein the controller is configured during operation to:
   receive the pressure variable from the pressure sensor at a first flow rate;
   determine if a possible leak condition is satisfied based on comparing the received pressure variable to a first threshold, the first threshold representing a possible leak condition, and
if the possible leak condition is satisfied:
   increase the motor speed of the flow generator to generate a second flow rate for the flow of gases;
   receive an updated pressure variable at the second flow rate;
   determine which of a possible leak condition, no leak condition, or definite leak condition is satisfied by comparing the updated pressure variable to the first threshold and/or a second threshold, the second threshold representing a definite leak condition, and
if the possible leak condition is again satisfied at the second flow rate:
   continually increase the motor speed of the flow generator by discrete increments to increase the flow rate of the gases in discrete intervals and re-comparing the updated pressure variable to the first and/or second thresholds after each discrete increment until either a definite leak condition or no leak condition is satisfied.

In a configuration, the disinfection kit or assembly comprises a disinfection tube and a filter, the disinfection tube being removably connected into the flow path in place of a removed humidification chamber to thereby fluidly couple a gases outlet associated with the flow generator to a main gases outlet component of the apparatus that connects to a patient circuit, and the filter being removably connected to an open port of the main gases outlet. By way of example, the filter may be a filter component. In one example, the filter component may be a filter cap or a cap with a filter, wherein the filter cap or cap engages with, in or over an open port of the main gases outlet.

In a configuration, the main gases outlet component of the apparatus comprises an elbow conduit, with the disinfection tube connected to first end of the elbow conduit, and the filter connected to the second end of the elbow conduit, such that the flow of gases from the flow generator flows through the disinfection tube, elbow conduit, and out the filter into atmosphere, during disinfection mode operation.

In a configuration, the first and/or second thresholds are configured for detecting a leak condition in the flow path that is indicative or representative of the filter of the disinfection kit or assembly being removed, not present, or at least partially dislodged from the main gases outlet during disinfection mode operation.

In a configuration, the leak condition represents a component of the disinfection kit or assembly is removed or disconnected from the flow path.

In a configuration, the disinfection kit or assembly comprises an Ozone module that may be coupled to the respiratory apparatus. The leak detection method may be configured to detect a dislodgement or misconnection of the ozone module that is part of the disinfection kit. The ozone module may be configured to pump ozone gas through the flow path of the respiratory apparatus in order to disinfect the flow path. The ozone module may be required to be used for a set period of time. The described leak detection method is configured to detect removal of the disinfection module (i.e. Ozone module) prior to the required time, and thereby cause an appropriate alarm.

In an aspect, the present disclosure relates to a respiratory apparatus that is configured to provide a flow of gases to a user for respiratory therapy comprising:
a motor-driven flow generator that is operable to generate a flow of gases;
a flow path for the flow of gases through the respiratory apparatus to a gases outlet of the respiratory apparatus;
a humidifier that is operable to heat and humidify the flow of gases, the humidifier comprising a removable humidification chamber in the flow path;
a flow rate sensor configured to sense the flow rate of the flow of gases in the flow path; and
a controller that is operatively connected to the flow generator and operable to control a flow rate of the flow of gases by controlling a motor speed of the flow generator, wherein the controller is configured during operation to execute a leak detection process for detecting leaks in the flow path by:
   receiving or calculating a flow rate variable indicative or representative of the flow rate of the flow of gases sensed by the flow rate sensor:
   initiating a first-stage leak evaluation comprising comparing the flow rate variable to a first leak threshold, the first leak threshold representing a possible leak condition; and
   initiating a second-stage leak evaluation if the flow rate variable is above the first leak threshold thereby indicating a possible leak, the second-stage leak evaluation comprising:
   increasing the motor speed of the flow generator from the current motor speed to a higher motor speed,
   comparing a new flow rate variable at the higher motor speed to the first leak threshold and a second leak threshold, the second leak threshold representing a definite leak condition, and then:
   generating a leak alarm and exiting the second-stage leak evaluation if the new flow rate variable is above the second leak threshold thereby confirming the possible leak as a definite leak; or
   exiting the second-stage leak evaluation without generating an alarm if the new flow rate variable is below the first leak threshold and second leak threshold thereby resolving the possible leak as no leak; or
   repeating the second-stage leak evaluation at a further higher motor speed or speeds until the possible leak is confirmed as a leak or resolved as no leak.

In another aspect, the present disclosure relates to a respiratory apparatus that is configured to provide a flow of gases to a user for respiratory therapy comprising:
a motor-driven flow generator that is operable to generate a flow of gases;
a flow path for the flow of gases through the respiratory apparatus to a gases outlet of the respiratory apparatus;
a humidifier that is operable to heat and humidify the flow of gases, the humidifier comprising a removable humidification chamber in the flow path;
a flow rate sensor configured to sense the flow rate of the flow of gases in the flow path; and
a controller that is operatively connected to the flow generator and operable to control a flow rate of the flow of gases by controlling a motor speed of the flow generator, wherein the controller is configured during operation to:
   receive or calculate a flow rate variable indicative or representative of the flow rate of the flow of gases sensed by the flow rate sensor at a first flow rate;
   determine if a possible leak condition is satisfied based on comparing the received flow rate variable to a first threshold, the first threshold representing a possible leak condition, and
if the possible leak condition is satisfied:
   increase the motor speed of the flow generator to generate a second flow rate for the flow of gases;
   receive an updated flow rate variable at the second flow rate;
   determine which of a possible leak condition, no leak condition, or definite leak condition is satisfied by comparing the updated flow rate variable to the first threshold and/or a second threshold, the second threshold representing a definite leak condition, and
if the possible leak condition is again satisfied at the second flow rate:
   continually increase the motor speed of the flow generator by discrete increments to increase the flow rate of the gases in discrete intervals and re-comparing the updated flow rate variable to the first and/or second thresholds after each discrete increment until either a definite leak condition or no leak condition is satisfied.

In another aspect, the present disclosure relates to a respiratory apparatus that is configured to provide a flow of gases to a user for respiratory therapy comprising:
a motor-driven flow generator that is operable to generate a flow of gases;
a flow path for the flow of gases through the respiratory apparatus to a gases outlet of the respiratory apparatus;
a humidifier that is operable to heat and humidify the flow of gases, the humidifier comprising a removable humidification chamber in the flow path;
a flow rate sensor configured to sense the flow rate of the flow of gases in the flow path; and
a controller that is operatively connected to the flow generator and operable to control a flow rate of the flow of gases by controlling a motor speed of the flow generator, wherein the controller is configured during operation to:
control the flow generator or motor speed of the flow generator to deliver the flow of gases at a configurable user set or preset flow rate in normal operation;
receive a flow rate variable representing or indicative of the flow rate of the flow of gases and either:
   continue normal operation if the flow rate variable is below a leak threshold thereby indicating no leak detected; or
   increase the motor speed or set flow rate of the flow generator by a predetermined or dynamically determined increment if the flow rate variable is above the leak threshold indicating a possible leak detected, and receive an updated flow rate variable at the new increased motor speed or set flow rate, and either:
      generate an alarm and/or freeze the motor speed if the updated flow rate variable is above the leak threshold thereby confirming the possible leak as a leak; or
      revert to normal operation of the flow generator at the original set flow rate if the flow rate variable is below the leak threshold, thereby resolving the possible leak is not a leak.

In an aspect, the present disclosure relates to a respiratory apparatus that is configured to provide a flow of gases to a user for respiratory therapy comprising:
a motor-driven flow generator that is operable to generate a flow of gases;
a flow path for the flow of gases through the respiratory apparatus to a gases outlet of the respiratory apparatus;
a humidifier that is operable to heat and humidify the flow of gases, the humidifier comprising a removable humidification chamber in the flow path and a heater plate that is operable to heat the humidification chamber;
a pressure sensor configured to generate a pressure variable representing a sensed pressure characteristic of the flow of gases in the flow path; and
a controller that is operatively connected to the flow generator and operable to control a flow rate of the flow of gases by controlling a motor speed of the flow generator, wherein the controller is configured during operation to execute a leak detection process for detecting leaks in the flow path by:
   initiating a first-stage leak evaluation comprising comparing the pressure variable to a leak threshold, the leak threshold representing a possible leak condition; and
   initiating a second-stage leak evaluation if the pressure variable is below the leak threshold thereby indicating a possible leak, the second-stage leak evaluation comprising:
   initiating a heater plate check process by applying a power or temperature process to the heater plate of the humidifier and evaluating the heating rate and/or cooling rate of the heater plate against on or more thresholds based on a temperature sensor of or associated with the heater plate to thereby determine the presence or absence of a humidification chamber being in thermal contact with the heater plate,
   generating a leak alarm and exiting the second-stage leak evaluation if the humidification chamber is determined by the heater plate check as being absent, thereby confirming the possible leak as a definite leak; or
   exiting the second-stage leak evaluation without generating an alarm if the humidification chamber is determined by the heater plate check as being present, thereby resolving the possible leak as no leak.

In an aspect, the present disclosure relates to a respiratory apparatus that is configured to provide a flow of gases to a user for respiratory therapy comprising:
a motor-driven flow generator that is operable to generate a flow of gases;
a flow path for the flow of gases through the respiratory apparatus to a gases outlet of the respiratory apparatus;
a humidifier that is operable to heat and humidify the flow of gases, the humidifier comprising a removable humidification chamber in the flow path and a heater plate that is operable to heat the humidification chamber;
a flow rate sensor configured to sense the flow rate of the flow of gases in the flow path; and
a controller that is operatively connected to the flow generator and operable to control a flow rate of the flow of gases by controlling a motor speed of the flow generator, wherein the controller is configured during operation to execute a leak detection process for detecting leaks in the flow path by:
   initiating a first-stage leak evaluation comprising comparing a flow rate variable indicative or representative of the flow rate of the flow of gases to a leak threshold, the leak threshold representing a possible leak condition; and
initiating a second-stage leak evaluation if the flow rate variable is above the leak threshold thereby indicating a possible leak, the second-stage leak evaluation comprising:
   initiating a heater plate check process by applying a power or temperature process to the heater plate of the humidifier and evaluating the heating rate and/or cooling rate of the heater plate against on or more thresholds based on a temperature sensor of or associated with the heater plate to thereby determine the presence or absence of a humidification chamber being in thermal contact with the heater plate,
   generating a leak alarm and exiting the second-stage leak evaluation if the humidification chamber is determined by the heater plate check as being absent, thereby confirming the possible leak as a definite leak; or
   exiting the second-stage leak evaluation without generating an alarm if the humidification chamber is determined by the heater plate check as being present, thereby resolving the possible leak as no leak.

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure.

FIG. 1 shows schematically a respiratory system configured to provide a respiratory therapy to a patient.

FIG. 2 is a front view of an example respiratory apparatus with a humidification chamber in position and a raised handle/lever.

FIG. 3 is a top view corresponding to FIG. 2.

FIG. 4 is a right-side view corresponding to FIG. 2.

FIG. 5 is a left side view corresponding to FIG. 2.

FIG. 6 is a rear view corresponding to FIG. 2.

FIG. 7 is a front left perspective view corresponding to FIG. 2.

FIG. 8 is a front right perspective view corresponding to FIG. 2.

FIG. 9 is a bottom view corresponding to FIG. 2.

FIG. 10 shows an example configuration of an air and oxygen inlet arrangement of a respiratory apparatus.

FIG. 11 shows another example configuration of an air and oxygen inlet arrangement of the respiratory apparatus.

FIG. 12 is a transverse sectional view showing further detail of the air and oxygen inlet arrangement of FIG. 11.

FIG. 13 is another transverse sectional view showing further detail of the air and oxygen inlet arrangement of FIG. 11.

FIG. 14 is a longitudinal sectional view showing further detail of the air and oxygen inlet arrangement of FIG. 11.

FIG. 15 is an exploded view of upper and lower chassis components of a main housing of the respiratory apparatus.

FIG. 16 is a front left side perspective view of the lower chassis of the main housing showing a housing for receipt of a motor/sensor module sub-assembly.

FIG. 17 is a first underside perspective view of the main housing of the respiratory apparatus showing a recess inside the housing for the motor/sensor module sub-assembly.

FIG. 18 is a second underside perspective view of the main housing of the respiratory apparatus showing the recess for the motor/sensor module sub-assembly.

FIG. 19A illustrates a block diagram of a control system interacting with and/or providing control and direction to components of a respiratory system.

FIG. 19B illustrates a block diagram of an example controller.

FIG. 20 illustrates a block diagram of a motor and sensor module.

FIG. 21 illustrates a sensing chamber of an example motor and sensor module.

FIG. 22 shows a summary flow diagram of a leak detection algorithm in accordance with an embodiment.

FIG. 23 shows a detailed flow diagram of an embodiment of a leak detection algorithm during a therapy mode or drying mode of the respiratory apparatus, and which is based on leak pressure thresholds.

FIG. 24A shows a graph of pressure versus flow rate, and illustrates examples of the leak thresholds associated with the leak detection algorithm of FIG. 23 in accordance with an embodiment and data relating to normal operation and leaks.

FIG. 24B shows another version of the graph of FIG. 24A that shows the leak thresholds without the data relating to normal operation and leaks.

FIG. 24C shows a close-up of the graph of FIG. 24A and shows particular leak threshold lines associated with implementation of the leak detection algorithm in a scenario of multiple iterations (A-C) of the algorithm in a scenario where the apparatus is operating normally with no leak detected.

FIG. 24D shows the graph of FIG. 24C and demonstrates an update or modification to a possible leak threshold line following the determination of normal operation with no leak detected.

FIG. 24E shows a close-up of the graph of FIG. 24a and shows particular leak threshold lines associated with the implementation of the leak detection algorithm in a scenario of multiple iterations (A, B) of the algorithm in a scenario where the apparatus is operating with a leak (e.g. chamber off), and follow-on iterations (C, D, E) once the leak has been resolved.

FIG. 25A shows a schematic example of the respiratory apparatus of the type shown in FIG. 2 operating in a disinfection mode with a disinfection kit installed in accordance with an embodiment.

FIG. 25B shows a detailed flow diagram of another embodiment of a leak detection algorithm for a disinfection mode of the respiratory apparatus and which is based on leak pressure thresholds.

FIG. 25C shows a graph of pressure versus flow rate, and illustrates examples of the leak thresholds associated with the leak detection algorithm of FIG. 25A for the disinfection mode in accordance with an embodiment and data relating to normal operation and leaks for the disinfection mode.

FIG. 26 shows a flow diagram of an embodiment of a block detection algorithm for a disinfection mode of the respiratory apparatus in accordance with an embodiment.

FIG. 27A shows a detailed flow diagram of another embodiment of a leak detection algorithm for the respiratory apparatus and which is based on leak flow rate thresholds.

FIG. 27B shows a graph of motor speed versus flow rate, and illustrates examples of leak thresholds associated with the leak detection algorithm of FIG. 27A in accordance with an embodiment and data relating to normal operation and leaks.

### DETAILED DESCRIPTION

Although certain examples are described below, those of skill in the art will appreciate that the disclosure extends beyond the specifically disclosed examples and/or uses and obvious modifications and equivalents thereof. Thus, it is intended that the scope of the disclosure herein disclosed should not be limited by any particular examples described below

### 1. Determining Leaks in the Flow Path of a Respiratory Apparatus Configured as an Unsealed System

The leak detection algorithm of this disclosure is applicable to a range of respiratory apparatus, and particularly unsealed systems that are operable or configurable to provide high flow therapy via an unsealed interface, such as a nasal cannula. High flow systems are inherently leaky i.e. due to the unsealed patient interface.

In an embodiment, the leak detection algorithm is configured to detect one or more specific types of leaks in the flow path.

In one configuration, the leak detection algorithm is configured to detect a leak in the flow path caused by the removal or disconnection (whether complete or partial) of a humidification chamber from the flow path.

In other configurations, the leak detection algorithm may be configured to detect any other significant leaks along the flow path, such as coupling or connection leaks between components in or along the flow path of the respiratory apparatus.

In other configurations, the leak detection algorithm may be configured to detect leaks at the outlet of the apparatus, for example detecting leaks caused by removal or disconnection (whether complete or partial) of a patient circuit (e.g. conduit or tubing) from the gases outlet of the apparatus.

In some configurations, the leak detection algorithm can be configured to monitor for one type of leak, or can be configured to monitor or sense for one or more different types of leaks, concurrently or one at a time periodically or serially.

In some configurations, the leak detection algorithm can be configured to monitor for particular or specific types of leaks during one or more different operating modes of the respiratory apparatus. In one example, the leak detection algorithm can be configured to operate during therapy modes, when a flow of gases is being delivered to a user or patient for respiratory therapy. In another example, the leak detection algorithm can be configured to operate during non-therapy modes such as, but not limited to, drying modes and disinfection modes. The types of leaks being detected may depend on the operating mode of the respiratory apparatus.

In an embodiment, the leak detection algorithm is configured to detect the removal or disconnection of the humidification chamber from the humidifier of the respiratory apparatus. The humidifier is an important feature and required when providing nasal high flow. Humidity assists in providing patient comfort and maintaining airway health of the patient. Humidification can also help in improving patient compliance to high flow therapy as it can improve patient comfort.

In one configuration, the leak detection algorithm is operable continuously across the full range of therapy operating flow rates of the apparatus and with any size patient interface, such as with both adult and neonatal or junior interfaces.

In unsealed systems, such as respiratory apparatus delivering nasal high flow, it can be difficult at low flow rate ranges for the system to accurately or easily distinguish when the humidification chamber has been removed from the humidifier or is not present in the flow path based on monitoring the characteristics of the flow of gases. There are chances of false positives when monitoring characteristics of the flow of gases to determine leaks such as humidification chamber disconnection or removal. At low flow rates, humidity is still important for patient therapy, especially for paediatric patients or neonatal patients, and therefore an accurate or reliable detection of humidification chamber removal is important.

In an embodiment, the respiratory apparatus may have a continuous control across its operating flow range of 0 L/min to 80 L/min. In an embodiment, the respiratory apparatus can operate selectively with multiple interfaces such as, but not limited to, small, medium, large adult cannula and small, medium, large junior cannula. In an embodiment, the respiratory apparatus is operable to provide flow therapy to a range of patients, e.g. adults, pediatric patients, or neonatal patients, in a single mode of operation, with the flow rate settings being adjusted for the specific patient. In an embodiment, the leak detection algorithm is provided with leak thresholds that are operable across the entire operating flow rate range, such that the same leak detection algorithm is continuously operable, regardless of the type of patient using the apparatus, and/or target flow rate setting, and/or type or size of patient interface (e.g. cannula) being used. For example, the leak detection algorithm, in some embodiments, is able to work across multiple flow rate ranges and with multiple different sized or types of patient interface, without modification of the leak thresholds or leak threshold functions.

As explained above, nasal high flow therapy delivered by a respiratory apparatus is very leaky. A nasal high flow system is unsealed and a low-pressure system. Leak detection, such as chamber disconnection detection, needs to be more sensitive for small changes in flow rate and pressure in the flow of gases generated by the system in the flow path. Typically, accurate leak detection is difficult because unsealed system alarm conditions and normal operation conditions converge, especially at lower flow rate ranges. For example, there is a higher chance of false positives when detecting leaks at low flow rates such as, but not limited to, at therapy flow rates for pediatric patients (e.g. below 15 L/min, and more particularly below 10 L/min). At these lower flow rate ranges, the flow rate and pressure conditions between normal operation and operation with a leak (e.g. humidification chamber removed) converge. This makes it difficult to discriminate between normal operation and when a leak has occurred.

There is a need for a leak detection method that is robust, fast acting and can determine a leak condition at low flow conditions with minimal or reduced false conditions.

### 2. Overview of Example Respiratory Apparatus

The methods and processes of determining leak will be described in the context of an example respiratory apparatus 10 that is configured or operable to provide nasal high flow therapy via an unsealed patient interface. This is intended as a non-limiting example. It will be appreciated that the leak detection methods and processes may be applied to other respiratory apparatus and/or to other modes of operation and/or modes of therapy delivered by such apparatus.

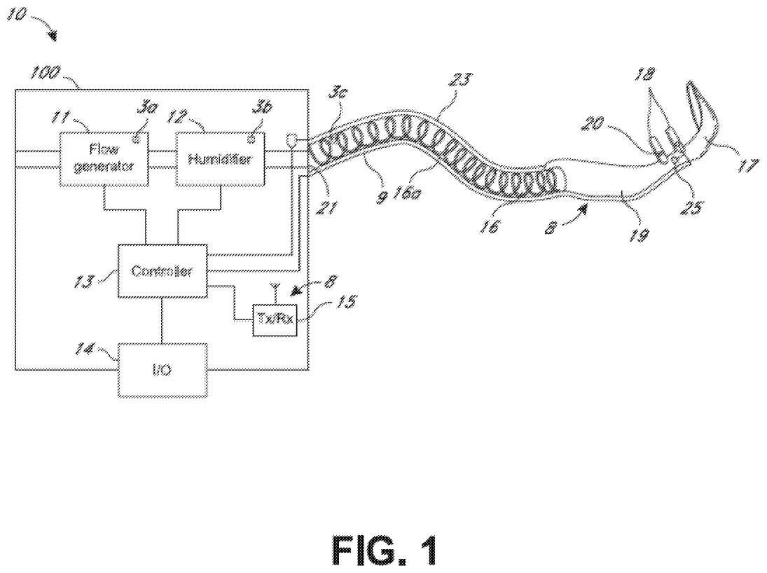

A schematic representation of the example respiratory apparatus 10 is provided in FIG. 1. The respiratory apparatus 10 can include a main device housing 100. The main device housing 100 can contain a flow generator 11 that can be in the form of a motor/impeller arrangement, a humidifier 12, a controller 13, and a user interface 14. The user interface 14 can include a display and input device(s) such as button(s), a touch screen, a combination of a touch screen and button(s), or the like. The controller 13 can include one or more hardware and/or software processors and can be configured or programmed to control the components of the system, including but not limited to operating the flow generator 11 to create a flow of gases for delivery to a patient, operating the humidifier 12 to humidify and/or heat the gases flow, receiving user input from the user interface 14 for reconfiguration and/or user-defined operation of the respiratory apparatus 10, and outputting information (for example on the display) to the user. The user can be a patient, healthcare professional, or others.

With continued reference to FIG. 1, a patient breathing conduit 16 can be coupled to a gases flow outlet 21 in the main device housing 100 of the respiratory apparatus 10, and be coupled to a patient interface 17, such as a non-sealing interface like a nasal cannula with a manifold 19 and nasal prongs 18. The patient breathing conduit 16 can also be a tracheostomy interface, or other unsealed interfaces.

The gases flow can be generated by the flow generator 11, and may be humidified, before being delivered to the patient via the patient breathing conduit 16 through the patient interface 17. The controller 13 can control the flow generator 11 to generate a gases flow of a desired flow rate, and/or one or more valves to control mixing of air and oxygen or other breathable gas. The controller 13 can control a heating element in or associated with the humidification chamber 12, to heat the gases to a desired temperature that achieves a desired level of temperature and/or humidity for delivery to the patient. The patient breathing conduit 16 can have a heating element 16a, such as a heater wire, to heat gases flow passing through to the patient. The heating element 16a can also be under the control of the controller 13.

The humidifier 12 of the apparatus is configured to combine or introduce humidity with or into the gases flow. Various humidifier 12 configurations may be employed. In one configuration, the humidifier 12 can comprise a humidification chamber that is removable. For example, the humidification chamber may be partially or entirely removed or disconnected from the flow path and/or apparatus. By way of example, the humidification chamber may be removed for refilling, cleaning, replacement and/or repair for example. In one configuration, the humidification chamber may be received and retained by or within a humidification compartment or bay of the apparatus, or may otherwise couple onto or within the housing of the apparatus.

The humidification chamber of the humidifier 12 may comprise a gases inlet and a gases outlet to enable connection into the gases flow path of the apparatus. For example, the flow of gases from the flow generator 11 is received into the humidification chamber via its gases inlet and exits the chamber via its gases outlet, after being heated and/or humidified.

The humidification chamber contains a volume of liquid, typically water or similar. In operation, the liquid in the humidification chamber is controllably heated by one or more heaters or heating elements associated with the chamber to generate water vapour or steam to increase the humidity of the gases flowing through the chamber.

In one configuration, the humidifier is a Passover humidifier. In another configuration, the humidifier may be a non-Passover humidifier. The leak detection algorithm may be configured to detect humidification chamber removal or disconnection from the flow path of the apparatus, whether the humidifier of the apparatus is a Passover or non-Passover humidifier.

In one configuration, the humidifier may comprise a heater plate, for example associated or within a humidification bay that the chamber sits on for heating. The chamber may be provided with a heat transfer surface, e.g. a metal insert, plate or similar, in the base or other surface of the chamber that interfaces or engages with the heater plate of the humidifier.

In another configuration, the humidification chamber may comprise an internal heater or heater elements inside or within the chamber. The internal heater or heater elements may be integrally mounted or provided inside the chamber, or may be removable from the chamber.

The humidification chamber may be any suitable shape and/or size. The location, number, size, and/or shape of the gases inlet and gases outlet of the chamber may be varied as required. In one configuration, the humidification chamber may have a base surface, one or more side walls extending up from the base surface, and an upper or top surface. In one configuration, the gases inlet and gases outlet may be position on the same side of the chamber. In another configuration, the gases inlet and gases outlet may be on different surfaces of the chamber, such as on opposite sides or locations, or other different locations.

In some configurations, the gases inlet and gases outlet may have parallel flow axes. In some configurations, the gases inlet and gases outlet may be positioned at the same height on the chamber.

The system 10 can use ultrasonic transducer(s), flow sensor(s) such as a thermistor flow sensor, pressure sensor(s), temperature sensor(s), humidity sensor(s), or other sensors, in communication with the controller 13, to monitor characteristics of the gases flow and/or operate the system 10 in a manner that provides suitable therapy. The gases flow characteristics can include gases concentration, flow rate, pressure, temperature, humidity, or others. The sensors 3a, 3b, 3c, 20, 25, such as pressure, temperature, humidity, and/or flow sensors, can be placed in various locations in the main device housing 100, the patient conduit 16, and/or the patient interface 17. The controller 13 can receive output from the sensors to assist it in operating the respiratory apparatus 10 in a manner that provides suitable therapy, such as to determine a suitable target temperature, flow rate, and/or pressure of the gases flow. Providing suitable therapy can include meeting a patient's inspiratory demand. In the illustrated embodiment the sensors are positioned in the housing of the apparatus.

The apparatus 10 can include a wireless data transmitter and/or receiver, or a transceiver 15 to enable the controller 13 to receive data signals 8 in a wireless manner from the operation sensors and/or to control the various components of the system 10. Additionally, or alternatively, the data transmitter and/or receiver 15 can deliver data to a remote server or enable remote control of the system 10. The system 10 can include a wired connection, for example, using cables or wires, to enable the controller 13 to receive data signals 8 from the operation sensors and/or to control the various components of the apparatus 10. The apparatus 10 may comprise one or more wireless communication modules. For example, the apparatus may comprise a cellular communication module such as for example a 3G, 4G or 5G module. The module 15 may be a modem that enables the apparatus to communicate with a remote server using an appropriate communication network. The communication may be two-way communication between the apparatus and a server or other remote system. The apparatus 10 may also comprise other wireless communication modules such as for example a Bluetooth module and/or a Wi-Fi module. The Bluetooth and/or WiFi module allow the apparatus to wirelessly send information to another device such as for example a smartphone or tablet or operate over a LAN (local area network).

The respiratory apparatus 10 may comprise a high flow therapy apparatus. High flow therapy as discussed herein is intended to be given its typical ordinary meaning, as understood by a person of skill in the art, which generally refers to a respiratory system delivering a targeted flow of humidified respiratory gases via an intentionally unsealed patient interface with flow rates generally intended to meet or exceed inspiratory flow of a user. Typical patient interfaces include, but are not limited to, a nasal or tracheal patient interface. Typical flow rates for adults often range from, but are not limited to, about fifteen litres per minute to about sixty litres per minute or greater. Typical flow rates for pediatric users (such as neonates, infants and children) often range from, but are not limited to, about one litre per minute per kilogram of user weight to about three litres per minute per kilogram of user weight or greater.

High flow therapy can also optionally include gas mixture compositions including supplemental oxygen and/or administration of therapeutic medicaments.

High flow therapy is often referred to as nasal high flow (NHF), humidified high flow nasal cannula (HHFNC), high flow nasal oxygen (HFNO), high flow therapy (HFT), or tracheal high flow (THF), among other common names. For example, in some configurations, for an adult patient ‘high flow therapy’ may refer to the delivery of gases to a patient at a flow rate of greater than or equal to about 10 litres per minute (10 LPM), such as between about 10 LPM and about 100 LPM, or between about 15 LPM and about 95 LPM, or between about 20 LPM and about 90 LPM, or between about 25 LPM and about 85 LPM, or between about 30 LPM and about 80 LPM, or between about 35 LPM and about 75 LPM, or between about 40 LPM and about 70 LPM, or between about 45 LPM and about 65 LPM, or between about 50 LPM and about 60 LPM. In some configurations, for a neonatal, infant, or child patient ‘high flow therapy’ may refer to the delivery of gases to a patient at a flow rate of greater than 1 LPM, such as between about 1 LPM and about 25 LPM, or between about 2 LPM and about 25 LPM, or between about 2 LPM and about 5 LPM, or between about 5 LPM and about 25 LPM, or between about LPM and about 10 LPM, or between about 10 LPM and about 25 LPM, or between 5 about 10 LPM and about 20 LPM, or between about 10 LPM and 15 LPM, or between about 20 LPM and 25 LPM. A high flow therapy apparatus with an adult patient, a neonatal, infant, or child patient, may deliver gases to the patient at a flow rate of between about 1 LPM and about 100 LPM, or at a flow rate in any of the sub-ranges outlined above.

High flow therapy can be effective in meeting or exceeding the patient's inspiratory demand, increasing oxygenation of the patient and/or reducing the work of breathing. Additionally, high flow therapy may generate a flushing effect in the nasopharynx such that the anatomical dead space of the upper airways is flushed by the high incoming gases flow. The flushing effect can create a reservoir of fresh gas available of each and every breath, while minimizing re-breathing of carbon dioxide, nitrogen, etc. High flow therapy can also increase expiratory time of the patient due to pressure during expiration. This in turn reduces the respiratory rate of the patient.

The patient interface for use in a high flow therapy can be a non-sealing interface to prevent barotrauma, which can include tissue damage to the lungs or other organs of the patients respiratory system due to difference in pressure relative to the atmosphere. The patient interface can be a nasal cannula with a manifold and nasal prongs, and/or an unsealed tracheostomy interface, or any other suitable type of patient interface.

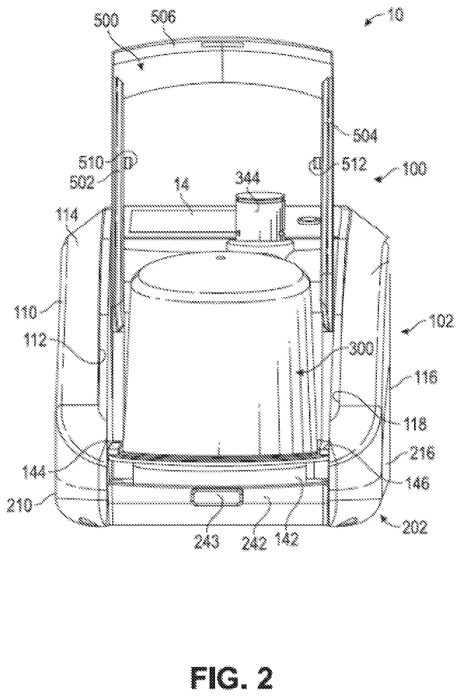

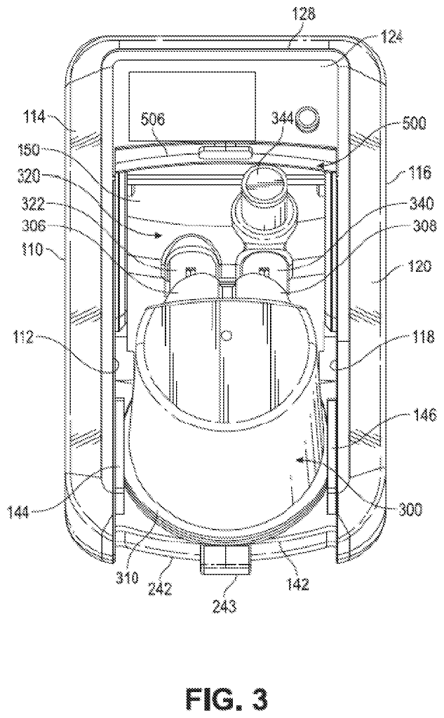

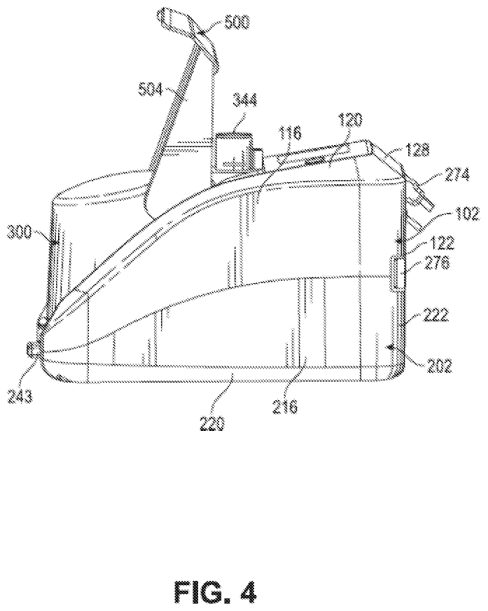

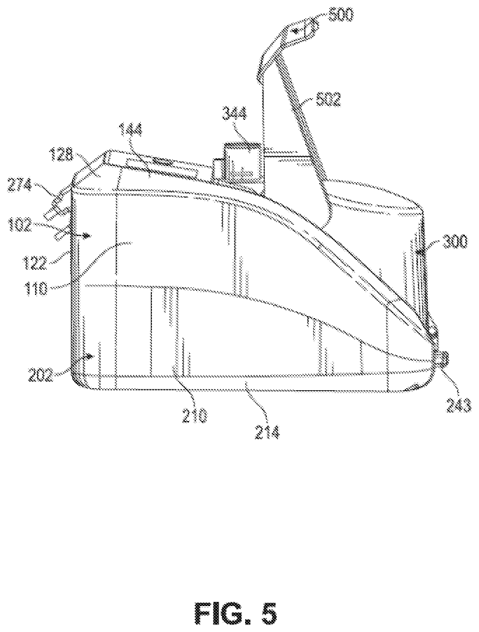

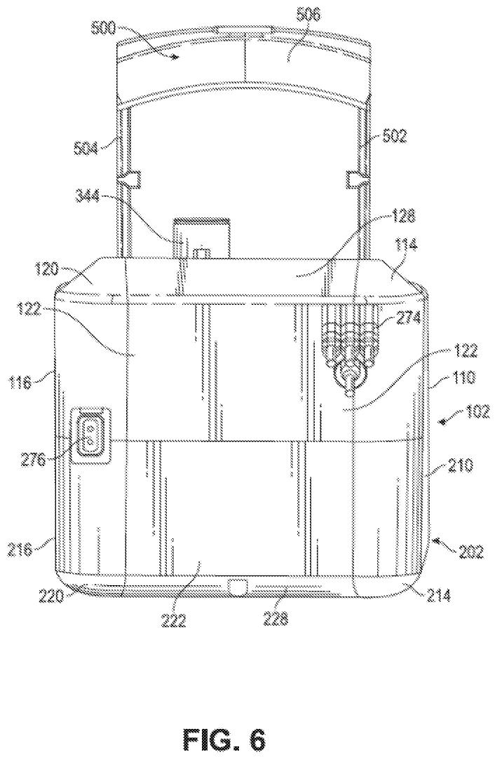

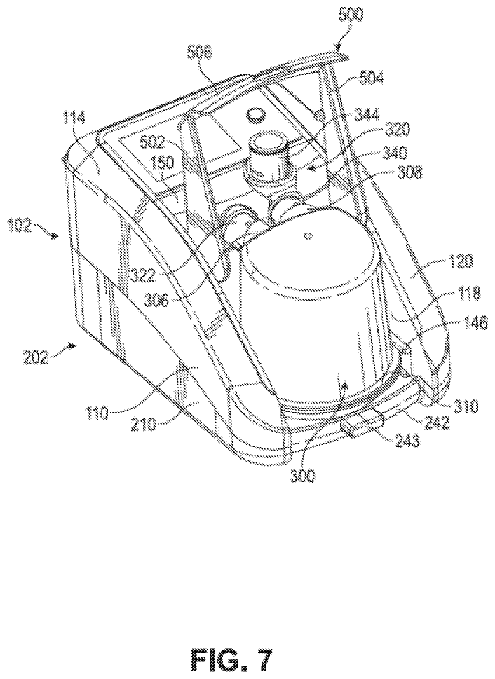

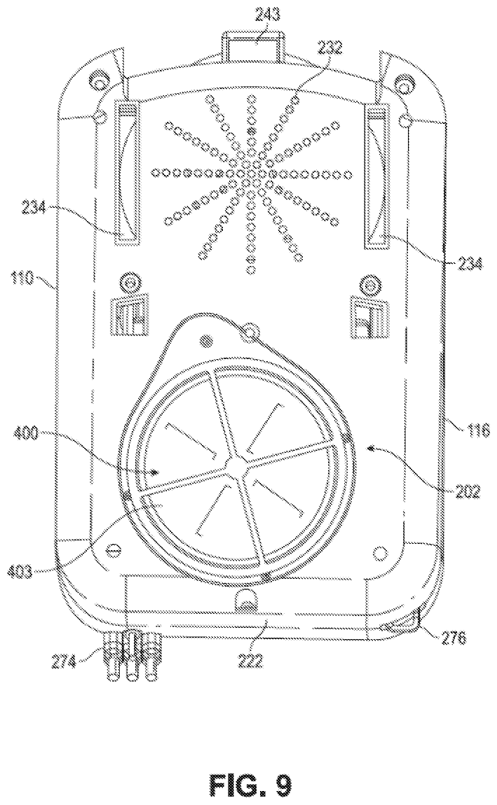

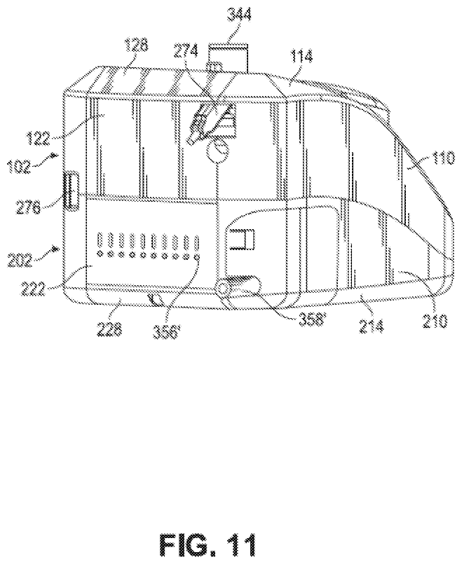

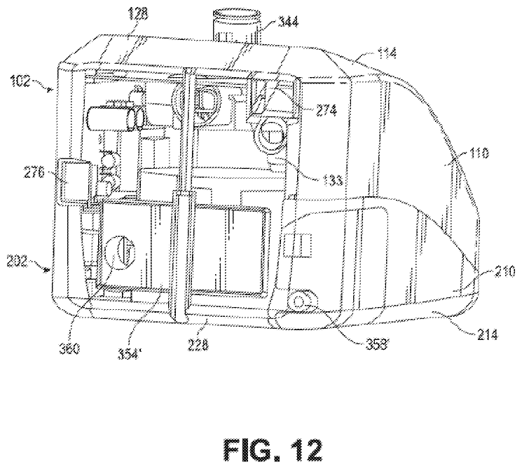

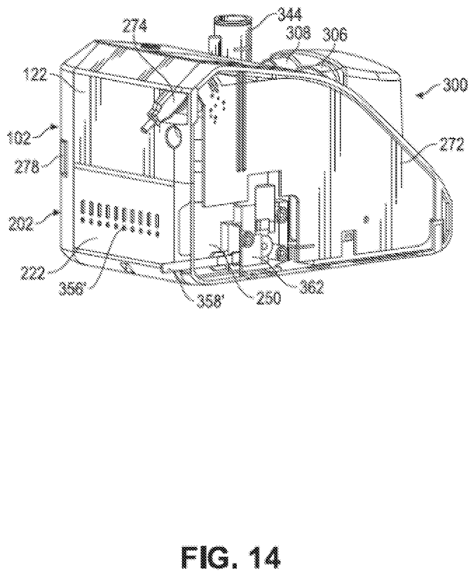

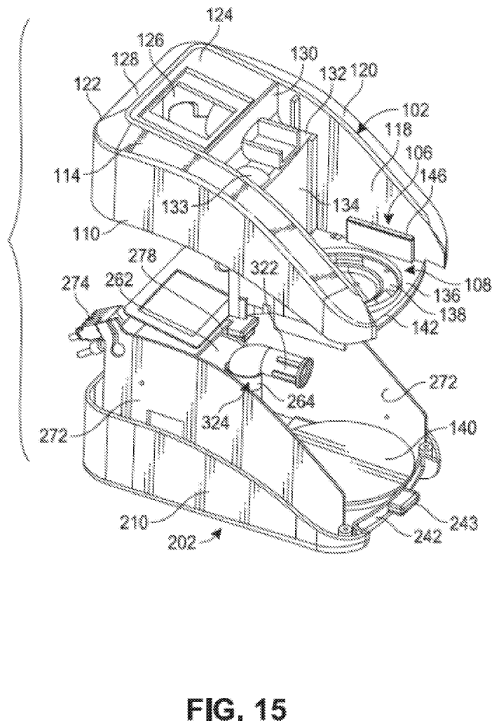

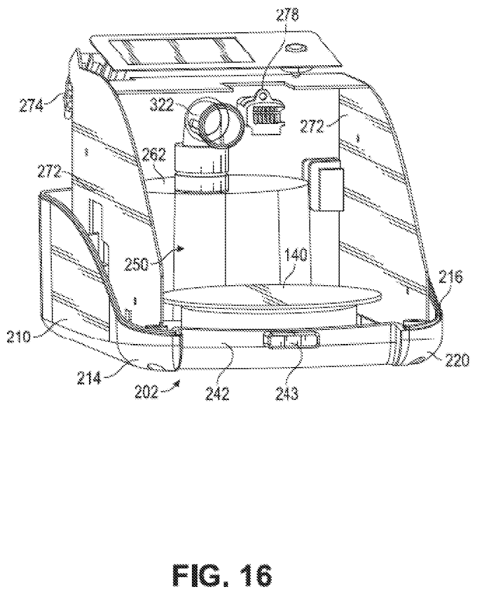

FIGS. 2 to 18 show an example respiratory apparatus of the respiratory apparatus 10 having a main housing 100. The main housing 100 has a main housing upper chassis 102 and a main housing lower chassis 202. The main housing upper chassis 102 has a peripheral wall arrangement 106 (see FIG. 15). The peripheral wall arrangement defines a humidifier or humidification chamber bay 108 for receipt of a removable humidification chamber 300. The removable humidification chamber 300 contains a suitable liquid such as water for humidifying gases that can be delivered to a patient.

In the form shown, the peripheral wall arrangement 106 of the main housing upper chassis 102 can include a substantially vertical left side outer wall 110 that is oriented in a front-to-rear direction of the main housing 100, a substantially vertical left side inner wall 112 that is oriented in a front-to-rear direction of the main housing 100, and an interconnecting wall 114 that extends between and interconnects the upper ends of the left side inner and outer walls 110, 112. The main housing upper chassis 102 can further include a substantially vertical right side outer wall 116 that is oriented in a front-to-rear direction of the main housing 100, a substantially vertical right side inner wall 118 that is oriented in a front-to-rear direction of the main housing 100, and an interconnecting wall 120 that extends between and interconnects the upper ends of the right side inner and outer walls 116, 118. The interconnecting walls 114, 120 are angled towards respective outer edges of the main housing 100, but can alternatively be substantially horizontal or inwardly angled.

The main housing upper chassis 102 can further include a substantially vertical rear outer wall 122. An upper part of the main housing upper chassis 102 can include a forwardly angled surface 124. The surface 124 can have a recess 126 for receipt of a display and user interface module 14. The display can be configured to display characteristics of sensed gas(es) in real time. The system can display the patient detection status of the patient interface. If the patient is not detected, the controller may not output or can stop outputting the respiratory rate value(s) and/or other parameters for display. The controller can also optionally output a message for display that no patient is detected at block 2708. An example of the message can be a “--” icon. An interconnecting wall 128 can extend between and interconnect the upper end of the rear outer wall 122 and the rear edge of the surface 124.

A substantially vertical wall portion 130 can extend downwardly from a front end of the surface 124. A substantially horizontal wall portion 132 can extend forwardly from a lower end of the wall portion 130 to form a ledge. A substantially vertical wall portion 134 can extend downwardly from a front end of the wall portion 132 and terminate at a substantially horizontal floor portion 136 of the humidification chamber bay 108. The left side inner wall 112, right side inner wall 118, wall portion 134, and floor portion 136 together can define the humidification chamber bay 108. The floor portion 136 of the humidification chamber bay 108 can have a recess 138 to receive a heater arrangement such as a heater plate 140 or other suitable heating element(s) for heating liquid in the humidification chamber 300 for use during a humidification process.

The main housing lower chassis 202 can be attachable to the upper chassis 102, either by suitable fasteners or integrated attachment features such as clips for example. The main housing lower chassis 202 can include a substantially vertical left side outer wall 210 that is oriented in a front-to-rear direction of the main housing 100 and is contiguous with the left side outer wall 110 of the upper chassis 102, and a substantially vertical right side outer wall 216 that is oriented in a front-to-rear direction of the main housing 100 and is contiguous with the right side outer wall 116 of the upper chassis 102. The main housing lower chassis 202 can further include a substantially vertical rear outer wall 222 that is contiguous with the rear outer wall 122 of the upper chassis 102.

The lower housing chassis 202 can have a lip 242 that is contiguous with the lip 142 of the upper housing chassis 102, and also forms part of the recess for receiving the handle portion 506 of the lever 500. The lower lip 242 can include a forwardly directed protrusion 243 that acts as a retainer for the handle portion 506 of the lever 500. Instead of the lever 500, the system can have a spring-loaded guard to retain the humidification chamber 300 in the humidification chamber bay 108.

An underside of the lower housing chassis 202 can include a bottom wall 230. Respective interconnecting walls 214, 220, 228 can extend between and interconnect the substantially vertical walls 210, 216, 222 and the bottom wall 230. The bottom wall 230 can include a grill 232 comprising a plurality of apertures to enable drainage of liquid in case of leakage from the humidification chamber 300 (e.g. from spills). The bottom wall 230 additionally can include elongated forward-rearward oriented slots 234. The slots 234 can additionally enable drainage of liquid in case of leakage from the humidification chamber 300, without the liquid entering the electronics housing. In the illustrated configuration, the slots 234 can be wide and elongate relative to the apertures of the grill 232 to maximize the drainage of liquid.

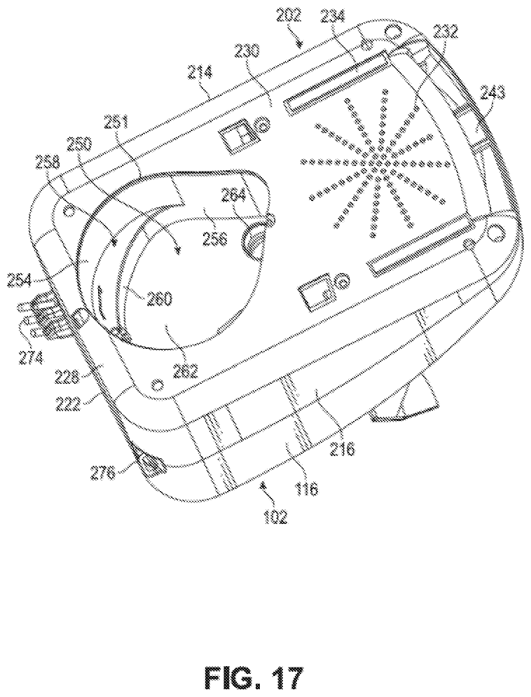

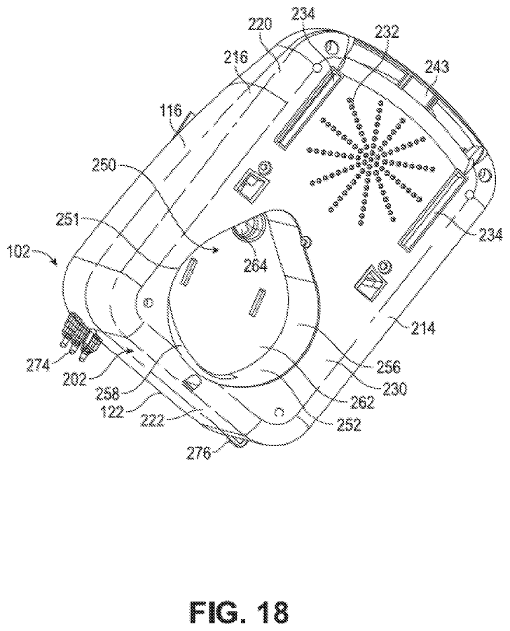

As shown in FIGS. 17 to 18, the lower chassis 202 can have a motor recess 250 for receipt of a motor and sensor module. The motor and sensor module may be non-removable from the main housing 100. The motor and sensor module can be removable from the main housing 100, as illustrated in FIGS. 17-18. A recess opening 251 can be provided in the bottom wall 230 adjacent a rear edge thereof, for receipt of a motor/sensor module. A continuous, gas impermeable, unbroken peripheral wall 252 can be integrally formed with the bottom wall 230 of the lower chassis 202 and extend upwardly from the periphery of the opening 251. A rearward portion 254 of the peripheral wall 252 has a first height, and a forward portion 256 of the peripheral wall 252 has a second height that is greater than the first height. The rearward portion 254 of the peripheral wall 252 terminates at a substantially horizontal step 258, which in turn terminates at an upper auxiliary rearward portion 260 of the peripheral wall 252. The forward portion 256 and upper auxiliary rearward portion 260 of the peripheral wall 252 terminate at a ceiling 262. All of the walls and the ceiling 262 can be continuous, gas impermeable, and unbroken other than the gases flow passage. Therefore, the entire motor recess 250 can be gas impermeable and unbroken, other than the gases flow passage.

The motor and sensor module can be insertable into the recess 250 and attachable to the lower chassis 202. Upon insertion of the motor and sensor module into the lower chassis 202, the gases flow passage tube 264 can extend through the downward extension tube 133 and be sealed by the soft seal.

The humidification chamber 300 can be fluidly coupled to the apparatus 10 in a linear slide-on motion in a rearward direction of the humidification chamber 300 into the chamber bay 108, from a position at the front of the housing 100 in a direction toward the rear of the housing 100. A gases outlet port 322 can be in fluid communication with the motor.

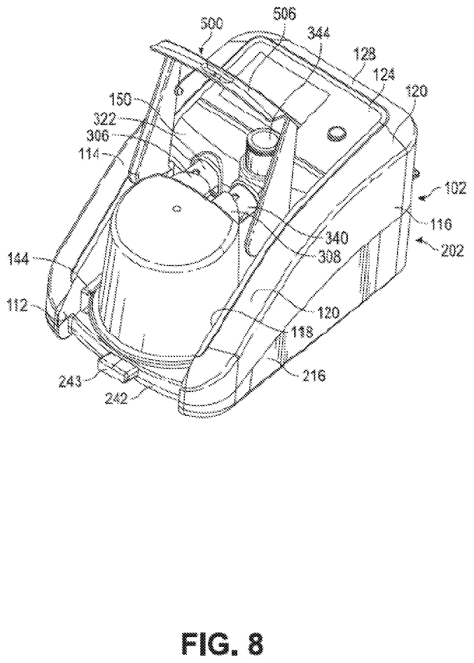

A gases inlet port 340 (humidified gases return) as shown in FIG. 8 can include a removable L-shaped elbow or ‘elbow conduit’ generally indicated at 320. The removable elbow can further include a patient outlet port 344 for coupling to the patient conduit 16 to deliver gases to the patient interface. The gases outlet port 322, gases inlet port 340, and patient outlet port 344 each can have soft seals such as O-ring seals or T-seals to provide a sealed gases passageway between the apparatus 10, the humidification chamber 300, and the patient conduit 16.

The humidification chamber gases inlet port 306 can be complementary with the gases outlet port 322, and the humidification chamber gases outlet port 308 can be complementary with the gases inlet port 340. The axes of those ports can be parallel to each other to enable the humidification chamber 300 to be inserted into the chamber bay 108 in a linear movement.

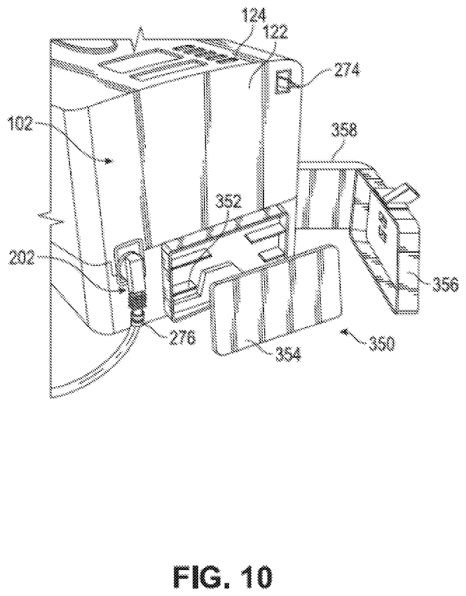

The respiratory apparatus can have air and oxygen (or alternative auxiliary gas) inlets in fluid communication with the motor to enable the motor to deliver air, oxygen (or alternative auxiliary gas), or a mixture thereof to the humidification chamber 300 and thereby to the patient. As shown in FIG. 10, the device can have a combined air/oxygen (or alternative auxiliary gas) inlet arrangement 350. This arrangement can include a combined air/oxygen port 352 into the housing 100, a filter 354, and a cover 356 with a hinge 358. A gases tube can also optionally extend laterally or in another appropriate direction and be in fluid communication with an oxygen (or alternative auxiliary gas) source. The port 352 can be fluidly coupled with the motor 402. For example, the port 352 may be coupled with the motor/sensor module 400 via a gases flow passage between the port 352 and an inlet aperture or port in the motor and sensor module 400, which in turn would lead to the motor.

The device can have the arrangement shown in FIGS. 11 to 14 to enable the blower to deliver air, oxygen (or alternative auxiliary gas), or a suitable mixture thereof to the humidification chamber 300 and thereby to the patient. This arrangement can include an air inlet 356′ in the rear wall 222 of the lower chassis 202 of the housing 100. The air inlet 356′ comprises a rigid plate with a suitable grill arrangement of apertures and/or slots. Sound dampening foam may be provided adjacent the plate on the interior side of the plate. An air filter box 354′ can be positioned adjacent the air inlet 356′ internally in the main housing 100, and include an air outlet port 360 to deliver filtered air to the motor via an air inlet port 404 in the motor/sensor module 400. The air filter box 354′ may include a filter configured to remove particulates (e.g. dust) and/or pathogens (e.g. viruses or bacteria) from the gases flow. A soft seal such as an O-ring seal can be provided between the air outlet port 360 and air inlet port 404 to seal between the components. The device can include a separate oxygen inlet port 358′ positioned adjacent one side of the housing 100 at a rear end thereof, the oxygen port 358′ for receipt of oxygen from an oxygen source such as a tank or source of piped oxygen. The oxygen inlet port 358′ is in fluid communication with a valve 362. The valve 362 can suitably be a solenoid valve that enables the control of the amount of oxygen that is added to the gases flow that is delivered to the humidification chamber 300. The oxygen port 358′ and valve 362 may be used with other auxiliary gases to control the addition of other auxiliary gases to the gases flow. The other auxiliary gases can include any one or more of a number of gases useful for gas therapy, including but not limited to heliox and nitric oxide.

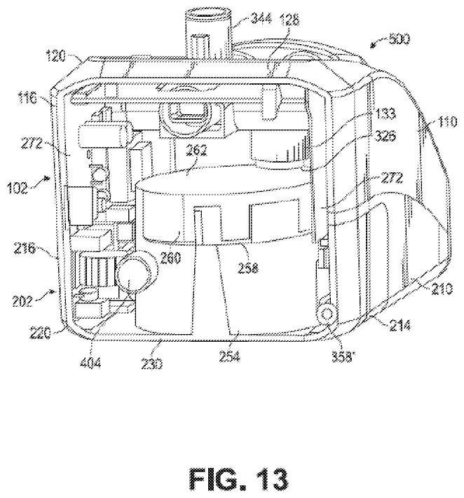

As shown in FIGS. 13 to 16, the lower housing chassis 202 can include suitable electronics boards, such as sensing circuit boards. The electronics boards can be positioned adjacent respective outer side walls 210, 216 of the lower housing chassis 202. The electronics boards can contain, or can be in electrical communication with, suitable electrical or electronics components, such as but not limited to microprocessors, capacitors, resistors, diodes, operational amplifiers, comparators, and switches. Sensors can be used with the electronic boards. Components of the electronics boards (such as but not limited to one or more microprocessors) can act as the controller 13 of the apparatus.

One or both of the electronics boards can be in electrical communication with the electrical components of the apparatus 10, including the display unit and user interface 14, motor, valve 362, and the heater plate 140 to operate the motor to provide the desired flow rate of gases, operate the humidification chamber 300 to humidify and heat the gases flow to an appropriate level, and supply appropriate quantities of oxygen (or quantities of an alternative auxiliary gas) to the gases flow.

The electronics boards can be in electrical communication with a connector arrangement 274 projecting from the rear wall 122 of the upper housing chassis 102. The connector arrangement 274 may be coupled to an alarm, pulse oximetry port, and/or other suitable accessories. The electronics boards can also be in electrical communication with an electrical connector 276 that can also be provided in the rear wall 122 of the upper housing chassis 102 to provide mains or battery power to the components of the device.

As mentioned above, operation sensors, such as flow, temperature, humidity, and/or pressure sensors can be placed in various locations in the respiratory apparatus, the patient breathing conduit 16, and/or cannula 17 such as shown in FIG. 1. The electronics boards can be in electrical communication with those sensors. Output from the sensors can be received by the controller 13, to assist the controller 13 to operate the respiratory apparatus 10 in a manner that provides optimal therapy, for example including meeting inspiratory demand. In the illustrated embodiment the sensors are positioned on electronic boards that are positioned within the housing. The sensors are encapsulated within the housing.

As outlined above, the electronics boards and other electrical and electronic components can be pneumatically isolated from the gases flow path to improve safety. The sealing also prevents water ingress.

### 2.1 Control System

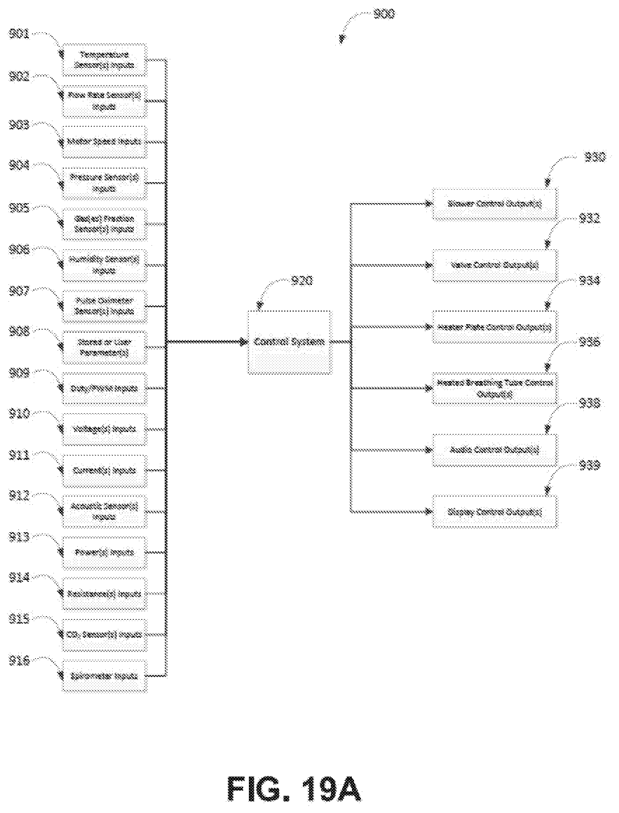

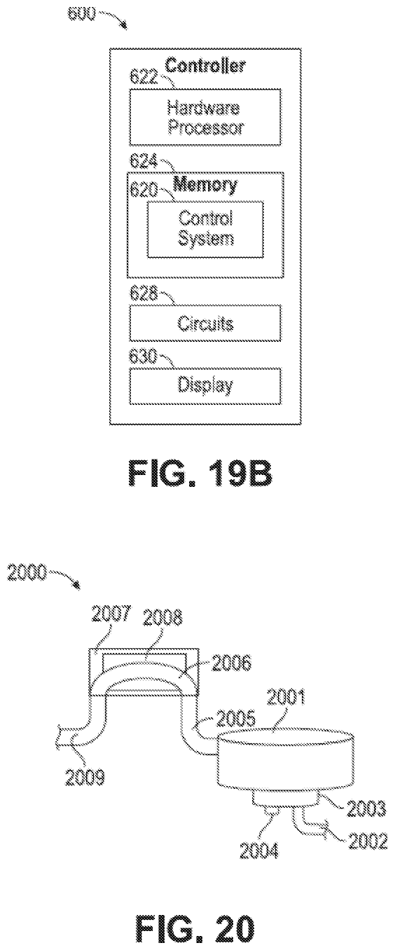

FIG. 19A illustrates a block diagram 900 of an example control system 920 (which can be the controller 13 in FIG. 1) that can detect patient conditions and control operation of the respiratory system including the gases source. The control system 920 can manage a flow rate of the gases flowing through the respiratory system as is the gases are delivered to a patient. For example, the control system 920 can increase or decrease the flow rate by controlling an output of a motor speed of the blower (hereinafter also referred to as a “blower motor”) 930 or an output of a valve 932 in a blender. The control system 920 can automatically determine a set value or a personalized value of the flow rate for a particular patient as discussed below. The flow rate can be optimized by the control system 920 to improve patient comfort and therapy.

The control system 920 can also generate audio and/or display/visual outputs 938, 939. For example, the flow therapy apparatus can include a display and/or a speaker. The display can indicate to the physicians any warnings or alarms generated by the control system 920. The display can also indicate control parameters that can be adjusted by the physicians. For example, the control system 920 can automatically recommend a flow rate for a particular patient. The control system 920 can also determine a respiratory state of the patient, including but not limited to generating a respiratory rate of the patient, and send it to the display, which will be described in greater detail below.

The control system 920 can change heater control outputs to control one or more of the heating elements (for example, to maintain a temperature set point of the gases delivered to the patient). The control system 920 can also change the operation or duty cycle of the heating elements. The heater control outputs can include heater plate control output(s) 934 and heated breathing tube control output(s) 936.

The control system 920 can determine the outputs 930-939 based on one or more received inputs 901-916. The inputs 901-916 can correspond to sensor measurements received automatically by the controller 600 (shown in FIG. 19B). The control system 920 can receive sensor inputs including but not limited to temperature sensor(s) inputs 901, flow rate sensor(s) inputs 902, motor speed inputs 903, pressure sensor(s) inputs 904, gas(s) fraction sensor(s) inputs 905, humidity sensor(s) inputs 906, pulse oximeter (for example, SpO₂) sensor(s) inputs 907, stored or user parameter(s) 908, duty cycle or pulse width modulation (PWM) inputs 909, voltage(s) inputs 910, current(s) inputs 911, acoustic sensor(s) inputs 912, power(s) inputs 913, resistance(s) inputs 914, CO₂ sensor(s) inputs 915, and/or spirometer inputs 916. The control system 920 can receive inputs from the user or stored parameter values in a memory 624 (shown in FIG. 19B). The control system 920 can dynamically adjust flow rate for a patient over the time of their therapy. The control system 920 can continuously detect system parameters and patient parameters. A person of ordinary skill in the art will appreciate based on the disclosure herein that any other suitable inputs and/or outputs can be used with the control system 920.

### Pressure Sensor or Sensors

In one configuration, the apparatus can have one or more pressure sensors. One or more pressure sensors may be provided to sense or measure a pressure characteristic of the flow of gases in the flow path of the apparatus and generated respective pressure variables, such as pressure sensor signals or data. The pressure sensors may include any type of suitable pressure sensor including, but not limited to, a gauge pressure sensor and/or an absolute pressure sensor.

A gauge pressure sensor may be configured to sense the gauge pressure of the flow of gases and generates a representative gauge pressure variable, such as a gauge pressure signal or pressure data. The gauge pressure may represent the pressure of the flow of gases in the flow path with reference or relative to atmospheric pressure. For example, the gauge pressure may represent the difference between the absolute pressure inside the flow path and the absolute pressure inside the housing (i.e. atmospheric or ambient pressure).

An absolute pressure sensor may be configured to sense the absolute pressure of the flow of gases and generates a representative absolute pressure variable, such as an absolute pressure signal or pressure data. The absolute pressure may represent the pressure of the flow of gases in the flow path with reference or relative to a vacuum.

As will be appreciated by a skilled person, the one or more pressure sensors that are configured to sense or measure a pressure characteristic of the flow of gases may be directly in or at least partially immersed in the main or bulk flow path of the flow of gases (e.g. the sensors may be part of or exposed to a sensor passage or sensor chamber that forms part of the main or bulk flow path), or directly in or at least partially immersed in a secondary or sample flow path that is operatively or fluidly connected to the main or bulk flow path, or otherwise operatively or fluidly couple or connected to the flow of gases in the flow path.

The pressure sensors for sensing pressure characteristics of the flow of gases may be independently mounted within the housing of the apparatus and electrically connected or otherwise in data communication with the controller or control system, or may be mounted or coupled to a sensor circuit board or other circuit board associated with the flow path of the flow of gases. In one configuration, the pressure sensors may be located or configured to sense the pressure of the flow of gases at a location along the flow path before (e.g. upstream of) the humidifier or humidification chamber. In another configuration, the pressure sensor(s) may be located or configured to sense the pressure of the flow of gases at a location along the flow path between the flow generator, e.g. blower, and humidifier chamber, for example at a location along the flow path between the blower outlet and humidifier chamber inlet (i.e. downstream of the blower and upstream of the humidifier chamber).

One or more pressures may also be provided for sensing other pressures associated with the apparatus, such as the ambient environment in which it is located. In one configuration, the apparatus may be provided with an ambient pressure sensor that is configured to sense or measure the ambient or atmospheric pressure of the local ambient environment in which the apparatus is located and generates a representative ambient pressure variable, such as an ambient pressure signal or pressure data. In one configuration, the ambient pressure sensor may be an absolute pressure located or positioned on or in the housing and which is configured to sense the ambient or atmospheric pressure of the environment the apparatus is located in.

In one configuration, the leak detection algorithm may be configured to receive a gauge pressure signal or data from a gauge pressure sensor representing the gauge pressure associated with the flow of gases in the flow path.

In another configuration, the leak detection algorithm may be configured to receive the gauge pressure signal or data from the gauge pressure sensor sensing the flow of gases, and an ambient pressure signal or data from an ambient pressure sensor. In such a configuration, the leak detection algorithm can be configured to utilise the ambient pressure data as an input to a correction algorithm or factor or function applied to the sensed gauge pressure signal or data. For example, the correction algorithm, factor or function may be configured to correct the sensed gauge pressure signal or data to take into account the impact of changing air density on the sensed gauge pressure signal or data. The leak detection algorithm may be configured to apply the correction algorithm, factor or function to the incoming pressure sensor data and then use the corrected gauge pressure signal or data in the remaining leak detection algorithm steps. In an alternative configuration, the gauge pressure signal or data may be pre-processed with the correction algorithm, factor or function prior to being input to the leak detection algorithm, and the leak detection algorithm may receive the corrected gauge pressure signal or data.

### 2.2 Controller

FIG. 19B illustrates a block diagram of an embodiment of a controller 600 (which can be the controller 13 in FIG. 1). The controller 600 can include programming instructions for detection of input conditions and control of output conditions. The programming instructions can be stored in the memory 624 of the controller 600. The programming instructions can correspond to the methods, processes and functions described herein. The programming instructions can be executed by one or more hardware processors 622 of the controller 600. The programming instructions can be implemented in C, C++, JAVA, or any other suitable programming languages. Some or all of the portions of the programming instructions can be implemented in application specific circuitry 628 such as ASICs and FPGAs.

The controller 600 can also include circuits 628 for receiving sensor signals. The controller 600 can further include a display 630 for transmitting status of the patient and the respiratory assistance system. The display 630 can also show warnings and/or other alerts. The display 630 can be configured to display characteristics of sensed gas(es) in real time or otherwise. The controller 600 can also receive user inputs via the user interface such as display 630. The user interface can include button(s) and/or dial(s). The user interface can comprise a touch screen.

### 2.3 Motor and Sensor Module

Any of the features of the respiratory system described herein, including but not limited to the humidification chamber, the flow generator, the user interface, the controller, and the patient breathing conduit configured to couple the gases flow outlet of the respiratory system to the patient interface, can be combined with any of the sensor modules described herein.

FIG. 20 illustrates a block diagram of the motor and sensor module 2000, which can be received by the recess 250 in the respiratory apparatus (shown in FIGS. 17 and 18). The motor and sensor module can include a blower 2001, which entrains room air to deliver to a patient. The blower 2001 can be a centrifugal blower.

One or more sensors (for example, Hall-effect sensors) may be used to measure a motor speed of the blower motor. The blower motor may comprise a brushless DC motor, from which motor speed can be measured without the use of separate sensors. For example, during operation of a brushless DC motor, back-EMF can be measured from the non-energized windings of the motor, from which a motor position can be determined, which can in turn be used to calculate a motor speed. In addition, a motor driver may be used to measure motor current, which can be used with the measured motor speed to calculate a motor torque. The blower motor may comprise a low inertia motor.

Room air can enter a room air inlet 2002, which enters the blower 2001 through an inlet port 2003. The inlet port 2003 can include a valve 2004 through which a pressurized gas may enter the blower 2001. The valve 2004 can control a flow of oxygen into the blower 2001. The valve 2004 can be any type of valve, including a proportional valve or a binary valve. In some embodiments, the inlet port does not include a valve.

The blower 2001 can operate at a motor speed of greater than 1,000 RPM and less than 30,000 RPM, greater than 2,000 RPM and less than 21,000 RPM, or between any of the foregoing values. Operation of the blower 2001 mixes the gases entering the blower 2001 through the inlet port 2003. Using the blower 2001 as the mixer can decrease the pressure drop that would otherwise occur in a system with a separate mixer, such as a static mixer comprising baffles, because mixing requires energy.

The mixed air can exit the blower 2001 through a conduit 2005 and enters the flow path 2006 in the sensor chamber 2007. A sensing circuit board with sensors 2008 can positioned in the sensor chamber 2007 such that the sensing circuit board is at least partially immersed in the gases flow. At least some of the sensors 2008 on the sensing circuit board can be positioned within the gases flow to measure gases properties within the flow. After passing through the flow path 2006 in the sensor chamber 2007, the gases can exit 2009 to the humidification chamber.

Positioning sensors 2008 downstream of the combined blower and mixer 2001 can increase accuracy of measurements, such as the measurement of gases fraction concentration, including oxygen concentration, over systems that position the sensors upstream of the blower and/or the mixer. Such a positioning can give a repeatable flow profile. Further, positioning the sensors downstream of the combined blower and mixer avoids the pressure drop that would otherwise occur, as where sensing occurs prior to the blower, a separate mixer, such as a static mixer with baffles, is required between the inlet and the sensing system. The mixer can introduce a pressure drop across the mixer. Positioning the sensing after the blower can allow the blower to be a mixer, and while a static mixer would lower pressure, in contrast, a blower increases pressure. Also, immersing at least part of the sensing circuit board and sensors 2008 in the flow path can increase the accuracy of measurements because the sensors being immersed in the flow means they are more likely to be subject to the same conditions, such as temperature and pressure, as the gases flow and therefore provide a better representation of the gases flow characteristics.

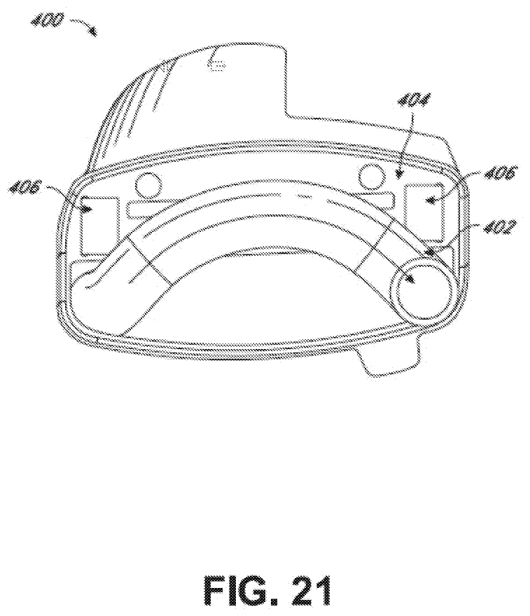

Referring to FIG. 21, the gases exiting the blower can enter a flow path 402 in a sensor chamber 400, which can be positioned within the motor and sensor module and can be the sensor chamber 2007 of FIG. 20. The flow path 402 can have a curved shape. The flow path 402 can be configured to have a curved shape with no sharp turns. The flow path 402 can have curved ends with a straighter section between the curved ends. A curved flow path shape can reduce pressure drop in a gases flow without reducing the sensitivity of flow measurements by partially coinciding a measuring region with the flow path to form a measurement portion of the flow path.

A sensing circuit board 404 with sensors, such as acoustic transmitters and/or receivers, humidity sensor, temperature sensor, pressure sensor(s), thermistor, and the like, can be positioned in the sensor chamber 400 such that the sensing circuit board 404 is at least partially immersed in the flow path 402. Immersing at least part of the sensing circuit board and sensors in the flow path can increase the accuracy of measurements because the sensors immersed in the flow are more likely to be subject to the same conditions, such as temperature and pressure, as the gases flow, and therefore provide a better representation of the characteristics of the gases flow. After passing through the flow path 402 in the sensor chamber 400, the gases can exit to the humidification chamber. Alternatively, one or more of the pressure sensor(s) may be provided on one or more separate circuit boards that are positioned or located so as to enable the pressure sensor(s) to measure or sense pressure characteristics associated with the flow of gases and/or ambient pressure.

The gases flow rate may be measured using at least two different types of sensors. The first type of sensor can comprise a thermistor, which can determine a flow rate by monitoring heat transfer between the gases flow and the thermistor. The thermistor flow sensor can run the thermistor at a constant target temperature within the flow when the gases flow around and past the thermistor. The sensor can measure an amount of power required to maintain the thermistor at the target temperature. The target temperature can be configured to be higher than a temperature of the gases flow, such that more power is required to maintain the thermistor at the target temperature at a higher flow rate.

The thermistor flow rate sensor can also maintain a plurality of (for example, two, three, or more) constant temperatures on a thermistor to avoid the difference between the target temperature and the gases flow temperature from being too small or too large. The plurality of different target temperatures can allow the thermistor flow rate sensor to be accurate across a large temperature range of the gases. For example, the thermistor circuit can be configured to be able to switch between two different target temperatures, such that the temperature of the gases flow will always fall within a certain range relative to one of the two target temperatures (for example, not too close but not too far). The thermistor circuit can be configured to operate at a first target temperature of about 50° C. to about 70° C., or about 66° C. The first target temperature can be associated with a desirable flow temperature range of between about 0° C. to about 60° C., or about 0° C. and about 40° C. The thermistor circuit can be configured to operate at a second target temperature of about 90° C. to about 110° C., or about 100° C. The second target temperature can be associated with a desirable flow temperature range of between about 20° C. to about 100° C., or about 30° C. and about 70° C.

The controller can be configured to adjust the thermistor circuit to change between at least the first and second target temperature modes by connecting or bypassing a resistor within the thermistor circuit. The thermistor circuit can be arranged as a Wheatstone bridge configuration comprising a first voltage divider arm and a second voltage divider arm. The thermistor can be located on one of the voltage divider arms. More details of a thermistor flow rate sensor are described in PCT Application Publication No. WO2018/052320, filed Sep. 3, 2017, which is incorporated by reference herein in its entirety.

The second type of sensor can comprise an acoustic sensor assembly. Acoustic sensors including acoustic transmitters and/or receivers can be used to measure a time of flight of acoustic signals to determine gases velocity and/or composition, which can be used in flow therapy apparatuses. In one ultrasonic sensing (including ultrasonic transmitters and/or receivers) topology, a driver causes a first sensor, such as an ultrasonic transducer, to produce an ultrasonic pulse in a first direction. A second sensor, such as a second ultrasonic transducer, receives this pulse and provides a measurement of the time of flight of the pulse between the first and second ultrasonic transducers. Using this time-of-flight measurement, the speed of sound of the gases flow between the ultrasonic transducers can be calculated by a processor or controller of the respiratory system. The second sensor can transmit and the first sensor can receive a pulse in a second direction opposite the first direction to provide a second measurement of the time of flight, allowing characteristics of the gases flow, such as a flow rate or velocity, to be determined. In another acoustic sensing topology, acoustic pulses transmitted by an acoustic transmitter, such as an ultrasonic transducer, can be received by acoustic receivers, such as microphones. More details of an acoustic flow rate sensor are described in PCT Application Publication WO2017/095241, filed Dec. 2, 2016, which is incorporated by reference herein in its entirety.

Readings from both the first and second types of sensors can be combined to determine a more accurate flow measurement. For example, a previously determined flow rate and one or more outputs from one of the types of sensor can be used to determine a predicted current flow rate. The predicted current flow rate can then be updated using one or more outputs from the other one of the first and second types of sensor, in order to calculate a final flow rate.

### 3. Example Embodiments of Two-Stage Leak Detection Process

The methods and processes of determining a leak in the flow path will be described in the context of the example respiratory apparatus 10 described above, which is configured or operable as a flow therapy apparatus to provide nasal high flow therapy via an unsealed patient interface. As explained earlier, the methods and processes may also be applied to other respiratory apparatus and/or to other modes of operation and/or modes of therapy delivered by such apparatus.

### 3.1 Overview of a Leak Detection Process

Referring to FIG. 22, an overview flow diagram of an embodiment of the leak detection process or algorithm 700 is shown, by way of example only. The leak detection process or method 700 in this example is implemented as an algorithm executed by a processor or controller of the apparatus. The process or method is referred to as the leak detection algorithm 700 in the following example.

The algorithm 700 operates or executes during operation of the respiratory apparatus 10, i.e. when it is generating a flow of gases. In some embodiments, the algorithm may operate when the patient is connected to the apparatus with their patient interface on or partially on to receive high flow therapy, or when the patient is disconnected from the apparatus with their patient interface off.

In one configuration, the algorithm 700 operates or executes during therapy modes and/or drying modes of the respiratory apparatus, when the apparatus is generating a flow of gases. In one embodiment, in these modes of operation, the leak detection algorithm is configured to detect leaks representing disconnection or removal of the humidification chamber from the flow path, but the algorithms may also be configured to detect other types of leaks or breaks in the flow path.

In another configuration, the algorithm 700 operates or executes during disinfection modes of the respiratory apparatus. As will be explained, during disinfection modes, the respiratory apparatus may be configured to generate a flow of gases that flows through a disinfection kit or assembly that is connected into the flow path of the apparatus. In one embodiment, in these disinfection modes of operation, the leak detection algorithm is configured to detect leaks representing disconnection or removal of one or more components of the disinfection kit or assembly, for example removal, disconnection, or dislodgement of the disinfection tube or conduit or a filter component of the disinfection kit or assembly from the flow path.

As explained, the leak detection algorithm 700 is configured to detect a leak in the flow path of the respiratory apparatus. In this example overview, the leak detection algorithm is explained with reference to detecting a leak in the flow path caused by partial or complete removal or disconnection of the humidification chamber of the humidifier from the flow path (i.e. a ‘chamber off’ or ‘chamber disconnect’ condition) during therapy or drying mode of the apparatus. However, as discussed above, it will be appreciated that the principles of operation of the leak detection algorithm may be applied to detect other types of leaks along the flow path, or leaks at the outlet of the flow path of the apparatus such as those caused by partial or complete disconnection of the patient conduit or tubing of a patient circuit from the gases outlet of the apparatus (i.e. a ‘tube off’ or ‘tube disconnect’ condition) during therapy or drying mode. As described, the patient circuit typically comprises a conduit or tubing connected at one end to the gases outlet of the apparatus and which delivers the flow of gases to the patients airways via a patient interface, such as a nasal cannula or the like, connected to the other end of the conduit or tubing. Other leaks that may be detected could include partial or complete disconnection of connections or couplings or conduits forming or defining the flow path within the apparatus, or leaks caused by holes or ruptures in along the flow path of the apparatus or patient circuit conduit or tubing, including holes or ruptures in the humidification chamber, for example. Typically, the leak detection algorithm is configured to detect substantial or large leaks in the flow path of the apparatus or patient circuit, i.e. leaks that are significantly or much greater than the leak at the unsealed patient interface of the patient circuit. Additionally or alternatively, as discussed above, the principles of operation of the leak detection algorithm may be applied to detect other leaks during disinfection mode of the respiratory apparatus, e.g. disconnection or dislodgement of a disinfection tube or filter component (e.g. filter cap or cap with a filter) of a disinfection kit or assembly from the flow path of the apparatus.

In one configuration, the leak detection algorithm 700 can detect leaks based on a known or measured relationship or model between the pressure of the flow of gases and the flow rate of the flow of gases. In particular, the pressure-flow rate characteristic curves or functions or models for the apparatus operating normally and with a leak (e.g. due to the humidification chamber being off or some other type of leak) are used to generate one or more leak thresholds or limits used in the algorithm. In this configuration, the leak thresholds are leak pressure thresholds or limits. As will be explained further later, the thresholds or limits may be a function of or based at least partly on flow rate and/or motor speed, and optionally one or more other operating characteristics or settings related to the apparatus or flow of gases.

In another configuration, the leak detection algorithm 700 can detect leaks based on a known or measured relationship or model between the flow rate of the flow of gases and the motor speed of the flow generator of the respiratory apparatus. In particular, the flow rate-motor speed characteristic curves or functions or models for the apparatus operating normally and with a leak (e.g. due to the humidification chamber being off or some other type of leak) are used to generate one or more leak thresholds or limits used in the algorithm. In this configuration, the leak thresholds are leak flow rate thresholds or limits. As will be explained further later, the thresholds or limits may be a function of or based at least partly on motor speed, and optionally one or more other operating characteristics or settings related to the apparatus or flow of gases.

In this example overview, the leak detection algorithm is explained with reference to detecting a leak in the flow path based on comparing sensed pressure in the flow path to leak pressure thresholds. The same principles of operation in this example overview of the leak detection algorithm may also apply to embodiments that detect leaks based on comparing sensed flow rates in the flow path to leak flow rate thresholds.

In this overview example, the leak detection algorithm is configured to compare a pressure variable (e.g. pressure signal or pressure data) from a pressure sensor or sensors that are configured to sense a pressure characteristic of the flow of gases in the flow path of the apparatus. In this example, the algorithm then compares the pressure variable to two leak pressure thresholds. A first threshold is a ‘possible leak threshold’ that is indicative of a possible or potential leak. A second threshold is a ‘definite leak threshold’ that is indicative of a definitive leak. As will be explained in further detail later, the algorithm employs these two leak thresholds, and a dual-stage approach to leak evaluation (e.g. no leak=‘chamber on’ or leak=‘chamber off’), to minimise or reduce the chances of false positives (false leak alarms) arising during operation. This is because at lower flow rates or in lower flow rate ranges of the operational flow rate range of the apparatus, the pressure-flow rate characteristic curves or data between normal operation with no leak (e.g. chamber on) and a leak (e.g. chamber off) begin to at least partially overlap or converge, such that it is difficult to discriminate accurately between a leak condition (e.g. chamber off) and no leak condition (e.g. chamber on).

In one configuration, the definitive leak threshold has a lower probability of false positives across at least a portion of its flow rate range, e.g. in at least a lower flow rate region. The possible leak threshold has a higher probability of false positives across at least a portion of its flow rate range (e.g. in at least a lower flow rate range), i.e. it is a more conservative threshold or limit that signifies a possible or potential leak condition or situation that requires further verification or determination.

In this example, if a potential or possible leak is determined in a first-stage leak evaluation of the leak detection algorithm, the algorithm moves to a second-stage leak evaluation to either confirm or definitively determine the leak (e.g. chamber is off), or definitely determine that there is no leak (i.e. chamber is on).

### First-Stage Leak Evaluation

Referring to the flow diagram of FIG. 22, the apparatus may commence or be operating in normal operation delivering a flow of gases to a user or patient at a configurable user set flow rate. The leak detection algorithm may start 701 operation immediately upon commencement of normal operation of the apparatus or after a preconfigured delay period to allow the operating characteristics time to settle. After starting, the leak detection algorithm continuously operates at a first-stage leak evaluation 702 during normal operation of the apparatus. In this first-stage leak evaluation 702, the pressure variable from a pressure sensor sensing the flow of gases in the flow path is compared against the definitive leak threshold and possible leak threshold to determine if one or more conditions are satisfied. While operating in the first-stage leak evaluation 702, the apparatus is considered as being in normal operation with no leak detected, i.e. a no leak condition or state is satisfied.

If the pressure variable is below the definitive leak threshold at the first-stage leak evaluation 702, a leak detected condition is satisfied and it is considered that a leak has been detected 703. In response to the leak detection, the process moves from the first-stage leak evaluation 702 to a leak detected state or stage 704 in which a leak alarm is generated.

One or more alarm actions may be executed in response to the generation of a leak alarm state 704. The alarm actions may include any one or more of the following:
generating an audible, visual, and/or tactile warning, alert, or notification indicating the leak has been detected. For example, a visual notification may be displayed on the display screen of the apparatus indicating that a leak has been detected and the nature of the leak and/or options to resolve the leak (e.g. ‘chamber off detected’).
freezing the motor speed of the flow generator at the current motor speed at which the leak was detected, or at the original motor speed associated with the original user set flow rate.
Controlling, via the flow generator, the flow of gases to a specific flow rate or pressure.
halting operation of the apparatus and/or flow generator, i.e. to halt the flow of gases entirely.
halting operation of the humidifier, i.e. turning off the heater of the humidifier.
reducing power applied to the heater of the humidifier. In one example, this may be achieved by changing the current, voltage, pulse width modulation (PWM) frequency, or other control or power signal applied to the heater of the humidifier.
reducing the power applied to the heater of the humidifier and heater wire of the tubing or conduit of the patient circuit. In one example, this may be achieved by reducing the patient dew point setting to a predefined lower setting, e.g. reducing to a dew point setting less than or equal to 31 degrees Celsius.
halting operation of the heater wire of the tubing or conduit of the patient circuit, i.e. turning off the heater wire.
reducing power applied to the heater wire of the tubing or conduit of the patient circuit. In one example, this may be achieved by changing the current, voltage, pulse width modulation (PWM) frequency, or other control or power signal applied to the heater wire of the tubing or conduit.
sending or transmitting data indicative of the leak alarm or leak alarm information to an external system or server or device over a data communications link or links via a communications module or network interface (e.g. modem) of the apparatus. In one example, the leak alarm information may be sent as part of therapy data, a therapy report, diagnostic report, or other data set or report sent or transmitted from the apparatus. Additionally or alternatively messages or alarms may be sent to a local device e.g. a smartphone or tablet. For example, the local device may be associated with a clinician or caregiver.
any combination of the above alarm actions.

In one configuration, if the pressure variable is below the possible leak threshold in the first-stage leak evaluation 702, a possible leak condition or state is satisfied, and the process moves to a second-stage leak evaluation 707 as shown at 706. In another configuration, if the pressure variable is below the possible leak threshold and above the definite leak threshold, the possible leak condition or state is satisfied, and the process moves to the second-stage leak evaluation 707.

The purpose of the second-stage leak evaluation 707 is to either confirm the possible leak condition as a definite leak, or discard the possible leak condition as being no leak. In one configuration, the second-stage leak evaluation 707 can repeat or loop until it either confirms that a leak is detected or no leak is detected, or until one or more other optional exit conditions are satisfied. The second-state leak evaluation 707 is discussed further below.

If neither of the above leak or possible leak conditions are satisfied, based on the evaluations of comparing the pressure variable to the definite and possible leak thresholds, the first-stage leak evaluation 702 considers that no leak is detected, i.e. the no leak condition or state remains satisfied. As shown at 705, the algorithm remains in the first-stage leak evaluation, continually evaluating the pressure variable against the leak thresholds for a definite leak or possible leak condition, as above.

### Second-Stage Leak Evaluation

In this configuration, the second-stage leak evaluation 707 comprises increasing the current operating motor speed by a dynamic or predetermined increment to a higher motor speed, and then re-assessing the updated pressure variable at the higher motor speed to the definite leak threshold and possible leak threshold to resolve the possible leak condition as either a definite leak or no leak.

If the re-assessment or evaluation at the higher motor speed does not resolve the possible leak condition in the second-stage leak evaluation 707, the motor speed is incremented again to yet another higher motor speed, and the updated pressure variable is again compared to the thresholds to determine a leak or no leak condition. This process of increasing the motor speed in increments, and re-assessing the pressure variable against the thresholds, repeats until the leak or no leak condition is satisfied, or until another optional exit condition is satisfied.

For example, in one configuration, the algorithm may exit the second-stage leak evaluation 707 if the motor speed is increased to a configurable or pre-set maximum motor speed, or the algorithm will only loop in the second-stage leak evaluation 707 while the motor speed is below a maximum motor speed threshold. Once that motor speed threshold is exceeded or the motor speed condition is not met, the algorithm may exit back to the first-stage leak evaluation 702 and normal operation, indicating that no leak has been detected.

In the second-stage leak evaluation 707, the possible leak condition is resolved as a definite leak if the updated pressure variable at the higher motor speed is below the definite leak threshold. If a definite leak is detected, the algorithm exits the second-stage leak evaluation 707 to the leak detected state 704 as shown at 708, and initiates one or more alarm actions, as described above.

In the second-stage leak evaluation 709, the possible leak condition is resolved as no leak if the updated pressure variable at the higher motor speed is above the definite leak threshold and above the possible leak threshold. If a no leak condition is satisfied, the algorithm exits to the first-stage leak evaluation 702 and normal operation as shown at 709.

Optionally, upon exiting the second-stage leak evaluation 707 with no leak detected 709, the algorithm may be configured to update or adjust one or more of the threshold limits as shown at 712. For example, in one configuration, the threshold adjustment 712 may comprise reducing or decrementing the possible leak detection threshold by a dynamic or predetermined amount to reduce or minimise the algorithm 700 repeating in an endless loop. In one configuration, the threshold adjustment 712 step may comprise reducing the possible leak threshold at or in the region of the original motor speed and/or measured flow rate associated with the normal operation prior to entering the second-stage leak evaluation 7097 In other configurations, the threshold adjustment 712 step may comprise reducing the possible leak threshold across the entire operating motor speed and/or flow rate range of the apparatus, or across a specific region of the operating motor speed and/or flow rate range of the apparatus, or at the current or latest set or target motor speed and/or flow rate operating upon exiting the second-stage leak evaluation 707.

In the second-stage leak evaluation 707, the possible leak condition is considered not resolved if neither of the leak detected 708 or no leak detection conditions 709 are satisfied based on the evaluations above. In one configuration, if neither of the leak 708 or no leak 709 conditions are satisfied within a predetermined time period, the second-stage leak evaluation 707 remains in the possible leak condition state and repeats as shown at 710 by again increasing the motor speed by an increment to yet another higher motor speed and then re-evaluating the new pressure variable against the thresholds again in an attempt to resolve the possible leak condition as either a leak condition 708 or no leak condition 709, as described above. This process repeats until the second-stage leak evaluation resolves the possible leak condition as a leak condition or no leak condition, or until another optional exit condition is satisfied (such as the maximum motor speed condition being reached, by way of example only).

### Leak Detected State—Resolving Alarm

If the algorithm 700 enters the leak detected state 704, a leak alarm and/or alarm actions may be implemented, as described above. During this alarm state 704, the algorithm can be configured to continually or periodically monitor the pressure variable of the flow of gases to check for whether the leak (e.g. chamber off) has been resolved. For example, the algorithm may be configured to continuously or periodically compare the pressure variable to the definite leak threshold or another threshold representing a leak resolved condition. If the pressure variable is above the threshold, a leak resolved condition is satisfied and the algorithm exits the leak detected state 704 back to the first-stage leak evaluation 702 and normal operation as shown at 711.

Upon resolution of the leak alarm (e.g. when the chamber has been re-installed or re-connected into the flow path of the apparatus), any alarm actions may be halted and the apparatus can return to normal operation at the previous user set flow rate settings. For example, if the motor speed was frozen as an alarm action, the motor speed is unfrozen and normal flow rate control is resumed via motor-speed control of the flow generator. If alarm notifications were initiated, whether audible and/or visual, such alarms are cleared or halted.

### General Features of the Leak Detection Algorithm

One or more of the following features or aspects of the leak detection algorithm 700 may optionally also apply during operation of the algorithm.

The leak detection algorithm 700 can operate continuously to check for leaks based on the real-time pressure variable data or signals received from a pressure sensor sensing a pressure characteristic of the flow of gases in the flow path during operation of the apparatus, including when it is operating to deliver flow therapy to a patient or user, e.g. in therapy mode. It will be appreciated that the leak detection algorithm may alternatively be configured to operate periodically or on an adhoc basis or at predetermined time periods based on the mode of operation or during particular time periods of a flow therapy session, e.g. during commencement or on start-up or other periods, for example.

In some configurations, the leak detection algorithm 700 can also be implemented or executed during non-therapy modes of the apparatus. By way of example, the leak detection algorithm may operate while the apparatus is operating in non-therapy modes, such as during drying modes and/or disinfection modes. In such modes, the apparatus is configured to generate flow of gases through the apparatus and/or connected patient circuit and/or disinfection kit assembly for the purpose of drying and/or disinfecting one or more components in the flow path of the apparatus and/or connected patient circuit, for example. In such non-therapy modes, depending on the type of mode, the leak detection algorithm may function to alarm or alert upon detection of a leak, such as chamber off or tube off or dislodgement of a component of the disinfection kit assembly, as if these components are removed or disconnected from the flow path, they will not be dried and/or disinfected and/or the drying mode and/or disinfection mode may not operate correctly.

The leak thresholds used in the leak detection algorithm for non-therapy modes may be the same or different to those used in therapy mode. In some non-therapy modes, such as drying and/or disinfection modes, the leak thresholds and/or functions may be calibrated and/or adjusted to account for changes or differences in the flow path of the apparatus compared to when it is operating in therapy mode. In one example, in some such non-therapy modes, the entire patient circuit (e.g. conduit and patient interface) or components of the patient circuit (e.g. conduit and interface, or interface only) may not be connected to the apparatus or be otherwise absent from the flow path. In another example, in some such non-therapy modes, the humidification chamber may be removed and a disinfection kit assembly installed into the flow path. In such situations, the leak thresholds and/or functions may be adjusted accordingly, as the absence of one or more such components or addition of new components may change the flow path characteristics.

In some configurations, the leak detection algorithm may be restricted to operating only during particular user set flow rate ranges and/or target motor speed ranges. For example, in one configuration, the algorithm may automatically operate for lower or low flow rate ranges and/or lower or low target motor speed ranges, where discriminating between a leak and no leak is more difficult for the reasons previously explained in unsealed systems. Such lower flow rate ranges may include below 30 L/min, or below 15 L/min, or below 10 L/min, by way of example only. The configuration of the dual-stage leak detection algorithm allows the algorithm to detect possible leaks when the apparatus is operating in the lower flow rate range and/or lower target motor speed ranges, and then incrementally increase the flow rate and/or motor speed until it is confirmed whether there is a leak or no leak, which is easier to discriminate as the flow rate and/or motor speed increases. By way of example only, the lower flow rate ranges may correspond to those used with a neonatal patient or patient's using a neonatal interface, e.g. a cannula that is smaller than an adult cannula. Typically, lower flow rates or flow rate ranges are used for neonatal patients, children, or other patients that need a lower flow rate for therapy, as compared to the higher flow rates typically used for adults.

In some configurations, the dual-stage leak detection algorithm comprising stages 702 and 707 may operate during lower flow rate ranges and/or motor speeds, and an alternative single-stage leak evaluation using a single leak threshold may operate at the remaining higher flow rate range and/or motor speed range of the operating flow rate range. For example, during lower flow rate settings and/or lower target motor speeds, the dual-stage leak detection approach 700 comprising checking the pressure variable of the flow of gases against the definite and possible leak thresholds may be implemented to reduce or minimise false positives where determination of an actual leak condition is more difficult. At the remaining higher flow rate settings, where the chances of false positives in leak detection are less likely, a single-stage leak evaluation based on comparing the pressure variable to a single leak threshold may be employed to determine a leak or no leak condition during operation without using second-stage assessment involving increasing the flow rate and/or motor speed. If the pressure variable is above the single leak threshold, normal operation continues, and if the pressure variable is below the single leak threshold, a leak detection condition is satisfied, and leak alarm generated.

In one configuration, the leak thresholds utilised in the leak detection algorithm 700 may be extracted based on measured, known or modelled relationships between pressure and flow rate for the apparatus in normal operation and with a leak (e.g. with chamber off, or tube off-depending on which type of leak the algorithm is configured to detect).

In one configuration, the threshold relationship may be represented as a function, equation, threshold curve, or look-up table, in which the pressure leak thresholds used in the leak evaluation is dependent on at least the flow rate of the flow generator at the time of the evaluation. For example, the specific possible and definite leak thresholds used in the various leak determination evaluations at the different stages of the algorithm are dependent or a function of the particular flow rate operating at the time of the evaluation.

In another configuration, the leak thresholds may be dependent on and/or a function of the particular flow rate and/or motor speed operating at the time of the threshold evaluation, and also one or more other inputs or sensed variables or characteristics associated with the apparatus or the flow of gases at the time of the evaluation. By way of example, the one or more other inputs or sensed variables or characteristics may include, but are not limited to, absolute pressure of the flow of gases, temperature of the flow of gases, oxygen concentration of the flow of gases, ambient pressure of environment.

In some configuration, the leak detection algorithm 700 may be operable over or across the entire operating flow rate range and/or any operating modes of the apparatus without modification. For example, the leak detection algorithm may operate continuously without modification of the threshold functions and/or process, regardless of the flow rate setting of the apparatus or changes in flow rate settings during a flow therapy session or between sessions.

In some configurations, the leak detection algorithm 700 may be configured to be agnostic of the mode of operation of the apparatus.

In some configurations, the leak detection algorithm 700 may be operable with multiple different types and/or sizes of patient interfaces without modification. For example, the leak detection algorithm may be configured to operate continuously without modification of the threshold functions and/or process, regardless of changes of the type or size of patient interfaces used in the patient circuit connected to the gases outlet of the apparatus. For example, the leak detection algorithm may be configured to be agnostic to the type or size (e.g. adult, junior, or paediatric cannula for example) of patient interface used in the flow therapy delivered by the apparatus.

In one example use case, the leak detection algorithm may be beneficial to an operating situation in which the apparatus is operating with a junior or paediatric cannula at low flow rates for a junior or neonatal patient. In such operational cases, where the flow rates are in the lower end of the operational range, the ability to distinguish or discriminate leaks is difficult, as previously explained. The leak detection algorithm provides a means to reliably and robustly determine leaks (e.g. chamber off or tube off conditions) in such operational conditions or settings.

In some configurations, the leak detection algorithm 700 may be configured to generate a preliminary notification or warning when detecting a possible leak condition at 706 as it moves to the second-stage leak evaluation 707. For example, the possible leak warning may be visual, audible, and/or tactile and may comprise generating or presenting a notification or indication on the display screen of the apparatus. By way of example, such a preliminary notification may provide information to the user as to the operation of the device, as it may noticeably loop in the second-stage evaluation incrementing in motor speed and/or flow rates in discrete intervals as it attempts to resolve the possible leak as a definite leak condition 708 or a no leak condition 709.

In some configurations, the leak detection algorithm 700 is configured to make comparison or evaluation decisions based on whether the pressure variable is above or below the relevant threshold(s) for specific or predetermined minimum evaluation time periods. For example, an evaluation decision is made based on whether the pressure variable is consistently above or below the threshold for a minimum evaluation time period to determine whether a particular condition is satisfied or not, and to decide which state or stage in the leak detection algorithm to progress to. This configuration can assist in avoiding noise or spikes in the pressure variable data or signal from unduly affecting the reliability of the decisions made by the algorithm.

In some configurations, the leak detection algorithm comprises specific or unique minimum evaluation time periods for each particular threshold comparison in the process flow. For example, the respective minimum evaluation times may be dependent on the specific threshold comparison or stage or state of the algorithm when conducting the comparison. In one configuration, the minimum evaluation times for threshold comparisons in the first-stage leak evaluations 702 are longer than those in the later second-stage leak evaluation 707. In such configurations, the shorter minimum evaluation times in the second-stage leak evaluation 707 can be beneficial as the second-stage leak evaluation 707 involves suspending normal operation of the apparatus and incrementing the motor speed in discrete intervals to resolve the possible leak condition as either a definite leak or no leak condition, and so shorter comparison evaluation times may reduce any prolonged interruption to normal operation of the apparatus.

In some configurations, the minimum evaluation time periods utilised in the leak detection algorithm may be uniform or the same for all or at least some threshold comparisons. In one example, the minimum evaluation times may be dependent on the particular stage or state of the algorithm. For example, the minimum evaluation times associated with the first-stage leak evaluations may be substantially the same, and those associated within the second-stage leak evaluations may be substantially the same.

The leak detection algorithm can be configured to detect one or more different types of leaks in or associated with the flow path of the apparatus or peripheral components connected to the flow path such as, but not limited to, a patient circuit (e.g. conduit and patient interface) connected to the gases outlet of the apparatus. In one configuration, the leak thresholds or leak threshold characteristics are tailored to the particular leak for detection. In other configurations, general leak thresholds or leak threshold characteristics may be utilised to detect one or more different types of leaks in the flow path.

In one configuration, the leak detection algorithm is configured to detect leaks in the form of or representing a humidification chamber being removed or disconnected from the flow path, whether partially or completely (e.g. ‘chamber off’). For example, in this configuration, the leak detection algorithm functions or operates as a humidification chamber connection status detector, i.e. whether the chamber is connected into the flow path of apparatus (i.e. ‘chamber on’), or the chamber is removed or disconnected from the apparatus (i.e. ‘chamber off’).

In another configuration, the leak detection algorithm is configured to detect leaks in the form of or representing disconnection of the patient circuit from the gases outlet of the apparatus (e.g. ‘tube off’). In this configuration, the leak detection algorithm functions or operates as a patient circuit or conduit connection status detector, i.e. whether the patient circuit or conduit is connected (i.e. ‘tube on’) or disconnected (i.e. ‘tube off”) from the gases outlet of the apparatus.

In another configuration, the leak detection algorithm is configured to detect, during operation of a disinfection mode, leaks in the form of or representing disconnection or dislodgement of one or more components of a disinfection kit assembly installed into the flow path of the respiratory apparatus.

In one configuration, the apparatus may be configured to operate a plurality of versions of the leak detection algorithm in parallel or interchangeably or alternatively or sequentially, each version of the leak detection algorithm being configured to detect a different type or nature of leak.

In the following sections 3.2-3.4 further example implementations and configurations of the two-stage leak detection algorithm described in the overview example above. Any one or more of the general features above may apply to any one or more of the examples below.

### 3.2 Detailed First Example of a Leak Detection Process-Leak Pressure Thresholds

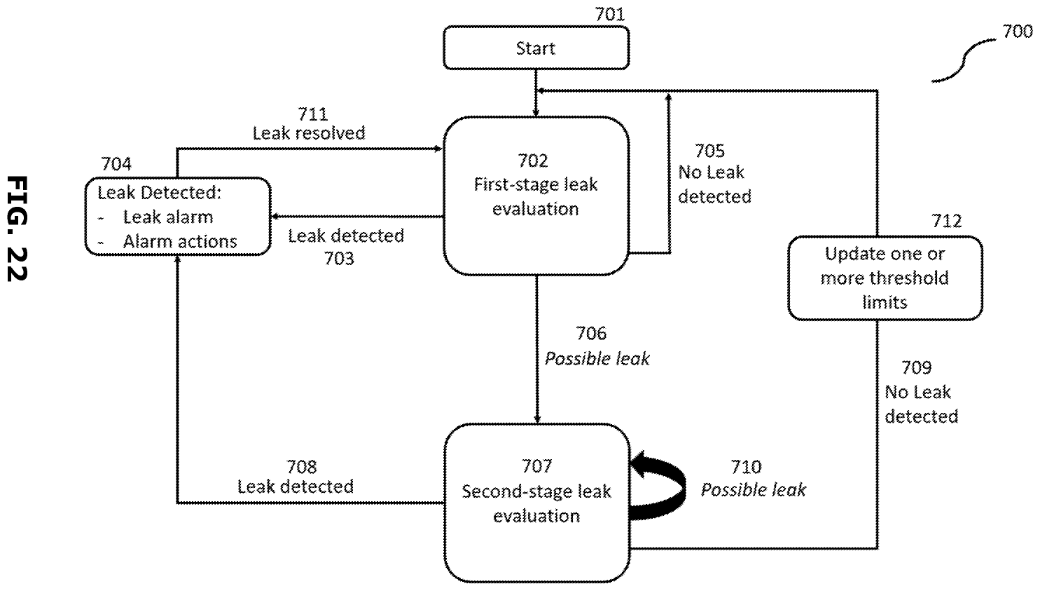

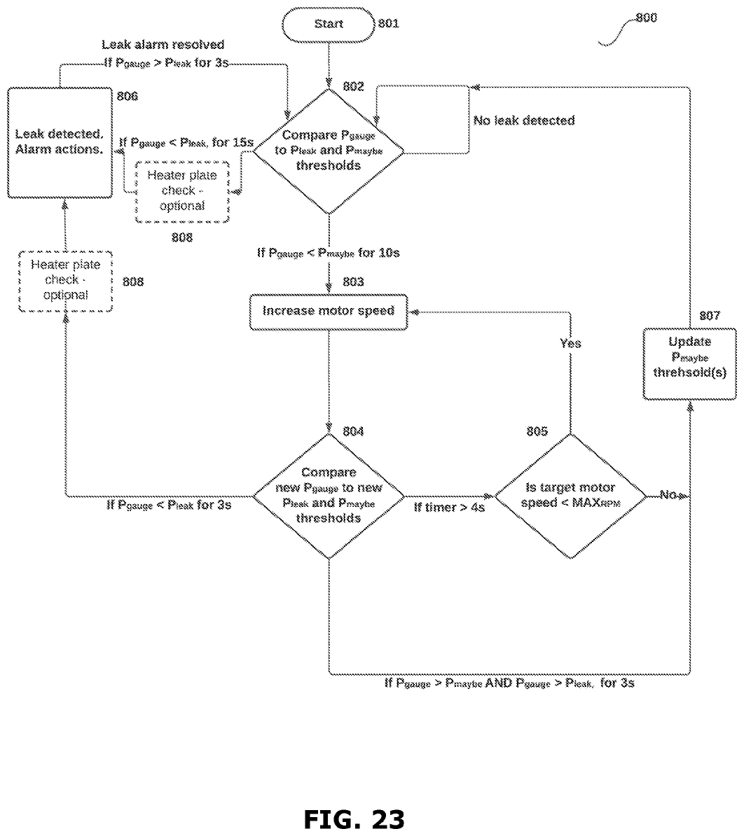

Referring to FIG. 23, a detailed flow diagram of an embodiment of the leak detection process or algorithm 800 is shown, by way of example only. This example algorithm 800 utilises the similar principles of operation of the summary algorithm 700 previously described and it will be appreciated that the features described in regard to either algorithm may also apply to the other algorithm.

This embodiment of the leak detection algorithm 800 is configured to detect leaks representing or caused by the humidification chamber being removed or disconnected (i.e. ‘chamber off’) from the flow path of the apparatus, such as when the chamber being removed from the humidifier chamber bay or compartment of the housing of the apparatus for cleaning, refilling, repair or replacement, or if the chamber is accidentally dislodged or disconnected from the flow path, for example.

In this embodiment, the leak detection algorithm 800 commences 801 during normal operation of the apparatus, for example when the apparatus is delivering a flow of gases at a configurable user set flow rate during a flow therapy session, e.g. while operating in a therapy mode. The configurable user set flow rate for the flow therapy session may depend on the patient and/or their prescribed therapy parameters or settings. As will be appreciated, a range of flow rate settings may be used for flow therapy, for adult and paediatric or neonatal patients. In some embodiments, the leak detection algorithm for determining the chamber connection status (e.g. chamber off) is operable across a full range of the operable flow rates, or in other embodiments is operable across a specific subset or subsets of the full flow rate range.

In this embodiment, the algorithm 800 receives a pressure variable representing the pressure signal or data sensed by a gauge pressure sensor that is configured to sense the gauge pressure of the flow of gases in the flow path. However, as will be appreciated, the pressure variable used by the algorithm may be from one or more other types of pressure sensors associated with the flow of gases in the flow path.

In this embodiment, the algorithm primarily has a first-stage leak evaluation 802, and a second-stage leak evaluation which comprises the steps and evaluations or determinations at 803, 804, and 805. The first-stage leak evaluation 802 is able to determine if there is a definite leak, possible leak, or no leak. The second-stage leak evaluation is configured to resolve a possible leak determination from the first-stage evaluation as either a definite leak or no leak.

In one embodiment, the algorithm 800 is suspended or delayed from operating until a predetermined delay period has expired from one or more particular events. These events may include any one or more of the following: commencement of a therapy session, commencement of normal operation or flow therapy control, resolution of a leak alarm or other alarms. For example, the algorithm 800 does not operate and cannot generate or trigger a leak alarm during the delay period after one or more selected or configured such events. In other embodiments, the algorithm 800 may be operable continuously and immediately at the start of a therapy session or when normal flow control resumes after a leak alarm has been resolved.

### First-Stage Leak Evaluation

In this embodiment, after any required delay period has expired, the algorithm 800 starts 801 by proceeding to the first-stage leak evaluation at step 802. In this first-stage leak evaluation 802, the gauge pressure variable is compared to definite and possible leak thresholds to determine the leak state of the apparatus, e.g. leak detected, possible leak, or no leak. In this embodiment, one or each condition may be considered as being satisfied or detected based on respective evaluation criteria being satisfied, as will be explained below. In this embodiment, the various evaluations based on comparing the gauge pressure to the one or more threshold(s), may occur concurrently or in parallel. In other configurations, the evaluations of which leak condition is satisfied may be configured to occur sequentially, or in a particular conditional order.

While operating in the first-stage leak evaluation 802, the apparatus can be considered as being in normal operation with no leak detected, i.e. in the no leak state. In this state or stage, the respiratory apparatus continues with normal operation and normal flow control in accordance with the set flow rate.

In this embodiment, the algorithm retrieves and/or receives input data representing the measured or sensed flow rate of the flow of gases, the measured or sensed gauge pressure variable, and the leak thresholds, for evaluation. The input data may be retrieved in accordance with a particular sampling frequency or continuously as the data becomes available from the sensors and/or main controller and/or memory of the apparatus. In one configuration, the input data may be a moving average based on a moving window of data. The window of data may be determined based on a configuration time period or number of data samples, for example. In one example, the input data may be a 10 second moving average of the sensed flow rate, sensed gauge pressure, and leak thresholds. In another configuration, the input data used by the leak detection algorithm may be the latest instantaneous data for the sensed flow rate, sensed gauge pressure, and leak thresholds.

In this embodiment, at step 802, one of the evaluations undertaken by the algorithm is to determine a definite leak condition. In this embodiment, to determine a definite leak condition, the algorithm compares the gauge pressure variable to a definite leak threshold Pₗₑₐₖ (which is a pressure threshold in this embodiment) representing a definite leak condition. If the gauge pressure variable P_{gauge} is less than the definite leak threshold Pₗₑₐₖ for a minimum evaluation time, then a leak condition is satisfied and the algorithm raises or generates a leak alarm at 806. In one configuration, the gauge pressure variable P_{gauge} must be below the definite leak threshold Pₗₑₐₖ for a minimum evaluation time period of 15 seconds, although it will be appreciated this time period may be adjusted or varied in other embodiments.

If the leak detection condition is satisfied based on the evaluation at step 802, the algorithm moves to the leak alarm or leak detected state or stage at 806. In the leak detected state 806, the algorithm may trigger one or more alarm actions, such as generating an audible, visual or tactile leak detection alarm, indicating the chamber is off or disconnected, and/or controlling or halting the flow rate or motor speed, or other control actions, as previously explained in regard to algorithm 700. In this embodiment, the algorithm 800 in the leak detected state 806 may be configured to execute one or more alarm actions. In this example embodiment, the algorithm 800 may execute any one or more of the following example alarm actions:
freezing the current target motor speed at which the leak was detected, such that the motor speed does not change from its present value while in the leak alarm state 806, until the leak is resolved.
raise or trigger an audible, visual and/or tactile alarm or notification to the user (e.g. message or notification on display of the respiratory apparatus and/or audible alarm via speaker or audio device of the respiratory apparatus).
Turn off or reduce the power applied to the humidifier, such as by turning off or reducing power applied to the heater plate of the humidifier. This can be advantageous in the leak condition, as there may be an increased safety risk to the user when the chamber is off, as the heater plate of the humidifier may be exposed, depending on the configuration of the humidifier of the apparatus.
Turn off or reduce the power applied to the heater wire of the patient circuit conduit connected to the apparatus.

Once in the leak detected state 806, the algorithm 800 may also be configured to determine or evaluate whether the leak alarm has been resolved, for example whether the chamber has been reconnected or installed back into the apparatus and/or flow path. In this embodiment, the algorithm at 806 continually evaluates or determines whether the leak has been resolved by comparing the pressure variable P_{gauge} to the definite leak threshold Pₗₑₐₖ or another alternative threshold specific to confirm leak resolution. In this example embodiment, if the gauge pressure variable P_{gauge} is over the Pₗₑₐₖ threshold for a minimum evaluation time period, which in this embodiment is 3 seconds but this could be adjusted in other embodiments, the leak alarm is considered as resolved. If the leak alarm is resolved, the algorithm returns to the first-stage leak evaluation 802 and normal operation or normal flow control in accordance with the set flow rate. If the gauge pressure variable does not exceed the Pₗₑₐₖ threshold for more than the minimum evaluation time period, then the algorithm remains in the leak detected state 806 and continues to check for resolution of the leak alarm.

In this embodiment, at step 802, one of the other evaluations undertaken by the algorithm is to determine a possible leak condition. In this embodiment, to determine a possible leak condition, the algorithm compares the gauge pressure variable P_{gauge} to a possible leak threshold P_{maybe} (which is a pressure threshold in this embodiment) representing a possible or potential leak condition. In this embodiment, if the gauge pressure variable P_{gauge} is below the possible leak threshold P_{maybe} for a minimum evaluation time period, a possible leak condition is satisfied and the algorithm moves to the second-stage leak evaluation (which commences at 803) to resolve the identified possible leak as a definite leak or no leak. In this embodiment, the minimum evaluation time period is 10 seconds, but this may be varied in different embodiments.

If neither of the definite leak or possible leak conditions are met based on the evaluations described above, the algorithm considers no leak is detected, and remains in the first-stage leak evaluation 802 in normal operation, i.e. no leak condition remains satisfied, and the apparatus continues with normal flow control.

### Second-Stage Leak Evaluation

If the algorithm detects a possible leak condition at 802, the algorithm moves to the second-stage leak evaluation, commencing at step 803. In this embodiment, before entering the second-stage leak evaluation to resolve the possible leak as a definite leak or no leak, the algorithm 800 is configured to store the measured flow rate (e.g. sensed by a flow rate sensor in the flow path or other sensing configuration) and the sensed pressure variable occurring before entering the second-stage leak evaluation at step 803. In one configuration, the stored values of the measured flow rate and stored sensed pressure variable may represent or be extracted from moving averages of these measured or sensed variables, as explained above. For example, in one configuration, the algorithm either receives during operation or calculates during operation moving averages of the measured flow rate and sensed pressure variable, from which it can extract and store values prior to entering the second-stage leak evaluation. By way of example, the stored values of measured flow rate and sensed pressure variable can be used in later algorithm steps involving updating and/or adjusting one or more of the leak threshold limits.

In this embodiment, the second-stage leak evaluation comprises increasing the current target motor speed by a predetermined increment or to a first or next pre-set higher motor speed at 803. In this embodiment, the algorithm is configured to increase and lock the motor speed at the first higher motor speed for a predetermined or maximum time period. In one configuration, the time period may be 4 seconds, but this may be varied in alternative embodiments. As will be explained below, during the or each iteration to a next higher motor speed, the higher motor speed is maintained for the configured time period (e.g. 4 seconds in this example) while the second-stage leak evaluations 804 are undertaken to attempt to resolve the possible leak as a definite leak condition or a no leak condition.

Once at the higher motor speed after the increase at 803, the algorithm 800 commences with one or more second-stage leak evaluations at 804. For example, the updated or new sensed pressure variable P_{gauge} at the higher motor speed is compared to new or updated definite Pₗₑₐₖ and possible P_{maybe} thresholds to determine whether the possible leak condition can be resolved as a definite leak condition or a no leak condition. Each condition may again have its own evaluation criteria to satisfy, based on comparing the sensed gauge pressure variable P_{gauge} to one or more of the leak thresholds, as will be described below.

In this embodiment, at step 804, one of the evaluations undertaken by the algorithm is to resolve the possible leak condition as a definite leak condition. In this embodiment, to determine a possible leak condition, the algorithm compares the updated sensed pressure variable P_{gauge} to an updated definite leak threshold Pₗₑₐₖ. In this embodiment, the new Pₗₑₐₖ threshold is updated or determined based at least partly on the new higher motor speed and/or flow rate of the flow of gases generated at the first higher motor speed. If the gauge pressure variable P_{gauge} is below the Pₗₑₐₖ threshold for a minimum evaluation time period (e.g. 3 seconds or another configurable time period), the possible leak condition is confirmed or resolved as a definite leak condition, and the algorithm progresses to the leak detected state 806 and generates a leak alarm or initiates one or more alarm actions as described previously.

In this embodiment, one of the other evaluations undertaken by the algorithm is to resolve the possible leak condition as a no leak condition. In this embodiment, to determine a no leak condition, the algorithm compares the updated sensed pressure variable P_{gauge} to updated possible leak threshold P_{maybe} and updated definite leak threshold Pₗₑₐₖ. As explained above, the updated thresholds may be extracted or determined based at least partly on the new higher motor speed and/or flow rate of the flow of gases generated at the first higher motor speed. If the updated sensed gauge pressure variable P_{gauge} is above the P_{maybe} threshold and above the Pₗₑₐₖ threshold for a minimum evaluation time (e.g. 3 seconds or another configurable time period), then the possible leak is resolved as no leak detected, and the algorithm reverts to the first-stage evaluation 802 and normal operation and/or flow control.

In this embodiment, upon exiting the second-stage leak evaluation at 804 (after resolving the possible leak is not a leak) and prior to reverting to step 802 and normal operation 801, the algorithm can optionally execute a threshold adjustment process at 807. In this embodiment, the threshold adjustment process 807 can be configured to adjust or reduce the P_{maybe} threshold to prevent the algorithm endlessly looping in the same scenario in future checks. In one configuration, the P_{maybe} threshold may be adjusted based on an adjustment or correction function 807 having one or more variables. By way of example, the variables of the adjustment or correction function may comprise the measured flow rate and sensed pressure variable values stored prior to entering the second-stage leak evaluation at step 803, as previously discussed. Additionally, the adjustment or correction function may include one or more further variables or constants. In one configuration, the adjustment or correction function may have a constant variable that is dependent on the stored measured flow rate, such that the magnitude of adjustment may vary depending on the stored measured flow rate. In one configuration, the constant variable may be configured to cause the adjustment or correction function to make smaller adjustments to the P_{maybe} threshold at lower flow rates or lower flow rate ranges.

In one embodiment, the threshold adjustment function 807 may comprise updating the P_{maybe} threshold based at least partly on a buffer value P_{buffer}. In one configuration, the P_{buffer} value may be at least partly dependent on the measured flow rate stored after exiting the first-stage 802. In another configuration, the P_{buffer} value may be a constant. In one configuration, the P_{maybe} threshold is extracted from a threshold function that is dependent on the measured flow rate and one or more constants. In this example configuration, the threshold adjustment function is configured to adjust the one or more constants of the threshold function based at least partly on the P_{buffer} value. In another example, the threshold adjustment function is configured to adjust the one or more constants of the threshold function based at least partly on the P_{buffer} value, and the P_{maybe} threshold value and measured pressure value stored after existing the first-stage 802.

In one embodiment, the threshold adjustment function 807 may be configured to adjust the P_{maybe} threshold based at least partly on a buffer value P_{buffer}. The P_{buffer} may be a variable that is configured to adjust the P_{maybe} threshold to prevent an infinite loop occurring in the leak detection algorithm. For example, the P_{buffer} is a value that is derived from a function or preconfigured to adjust the P_{maybe} threshold so as to prevent the leak detection algorithm from looping through the process of: (1) detecting a possible leak in the first-stage evaluation 802, (2) incrementing the motor speed (once or more) and determining the possible leak is not a leak in the second-stage evaluation 803-805, (3) exiting to the first-stage evaluation with the same P_{maybe} threshold to detect a possible leak again, and repeating (1)-(3) in an infinite loop without the flow path characteristics changing. In this embodiment, the P_{maybe} threshold is progressively adjusted or tuned as the leak detection algorithm operates.

In some configurations of the adjustment or correction function 807, the P_{maybe} threshold at the discrete measured flow rate sensed prior to entering the second-stage leak evaluation at 803 may be reduced or decremented by a calculated or predetermined amount. In other configurations, the entire P_{maybe} threshold function or threshold curve may be reduced or decremented by a calculated or predetermined amount across the entire operating flow rate range.

Reverting to the second-stage leak evaluations at 804, if neither of the definite leak or no leak conditions are satisfied based on the evaluations described above, the possible leak condition is considered as unresolved. In this embodiment, if the possible leak condition is unresolved for a predetermined time period based on a timer (e.g. 4 seconds in this example, but could be another configurable time period), then the second-stage leak evaluation process repeats, but at a further higher motor speed.

In this embodiment, if the timer expires and the possible leak condition is unresolved, the algorithm exits 804 and initiates a motor speed check 805. In this embodiment, the motor speed check 805 comprises comparing the current target motor speed against a pre-set or configurable motor speed threshold (MAX_{RPM}), before repeating or looping back to the start 803 of the second-stage leak evaluation.

In this embodiment, if the current target motor speed is equal to or above the motor speed threshold MAX_{RPM} at the motor speed check 805, the algorithm exits the second-stage leak evaluation and resolves the possible leak as a no leak condition, and reverts to the first-stage 802 and normal operation via the threshold adjustment process 807 previously explained. The motor speed check 805 provides an additional exit condition for the algorithm, and prevents the algorithm from endlessly looping in the second-stage evaluation and/or from increasing the motor speed beyond safety or component thresholds.

If the current target motor speed is below the motor speed threshold MAX_{RPM} at the motor speed check 805, the algorithm loops back to step 803, and again increases the first higher motor speed to a second or next higher motor speed, and repeats the evaluations at 804 again, in an attempt to resolve the possible leak as a definite leak or no leak condition.

As shown, the second-stage comprises continuously or repeatedly incrementing the motor speed by a predetermined amount or to a next predetermined higher motor speed, and then undertaking evaluations at 804 until the leak condition is satisfied, the no leak condition is satisfied, or the motor speed check fails at 805. As will be appreciated, depending on the evaluations, the second stage of the algorithm may complete once, twice or multiple times, prior to exiting back to 802 (no leak) or 806 (leak detected).

At step 803, it will be appreciated that the increments in motor speed may be pre-set or configurable increments or may increment to the next highest motor speed in a pre-set or configurable series or array of preselected discrete higher motor speeds. By way of example only, in one configuration, the motor speed increase at step 806 is based on incrementing the current motor speed to the next highest motor speed in the series or array comprising motor speeds of 5000 rpm, 6750 rpm, 8500 rpm, 10250 rpm, and 12000 rpm, such that the motor speed increases by 1750 rpm each cycle or loop of the second-stage leak evaluation. In this example, there are five possible discrete motor speed stages in which the pressure variable is compared against the P_{maybe} and Pₗₑₐₖ thresholds, but this may be varied to more or less stages as desired

In this embodiment, the leak detection algorithm 800 repeats once it returns to the first-stage leak evaluation and normal operation at 802. In one configuration, the leak detection algorithm continually operates as it receives updated real-time sensed pressure variable data from the pressure sensor(s). In other configurations, the leak detection algorithm may be configured to operate periodically. As previously discussed, in some configurations, upon the apparatus commencing or re-commencing normal operation, the leak evaluation comparisons against the leak thresholds may be suspended or delayed for a predetermined delay period after specific events. Such events may include, but are not limited to, commencement of a new therapy session or new user set flow rate, resolution of a prior leak alarm at 806, or otherwise when normal operation or flow control is resumed at 802, for example after a leak alarm has been resolved or after exiting the second-stage leak evaluation with no leak detected.

### Leak Thresholds

An example of the leak thresholds associated with the leak detection algorithm 800 will now be further explained. As discussed, the definite leak threshold Pₗₑₐₖ and possible leak threshold P_{maybe} for each leak evaluation comparison may be based on the measured flow rate and/or motor speed operating at the time of the evaluation comparison. In one configuration, the Pₗₑₐₖ and P_{maybe}, thresholds may be dependent on or a function of at least the sensed flow rate of the flow of gases and/or motor speed of the flow generator, and optionally one or more other variables or operating characteristics of the apparatus and/or flow of gases. The Pₗₑₐₖ and P_{maybe} thresholds may be functions, threshold curves or lines, or provided in the form of a look-up table. The thresholds may be extracted from the function, threshold curves or lines, or look-up tables based on at least the current sensed flow rate and/or motor speed of the flow generator, and any other optional variables, at each stage of the leak detection algorithm.

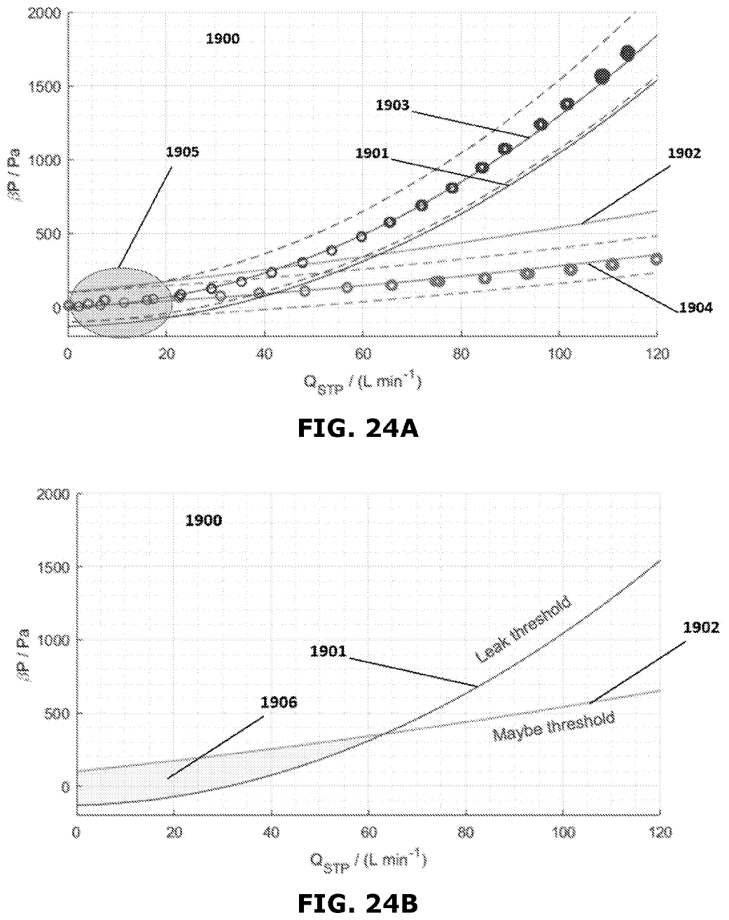

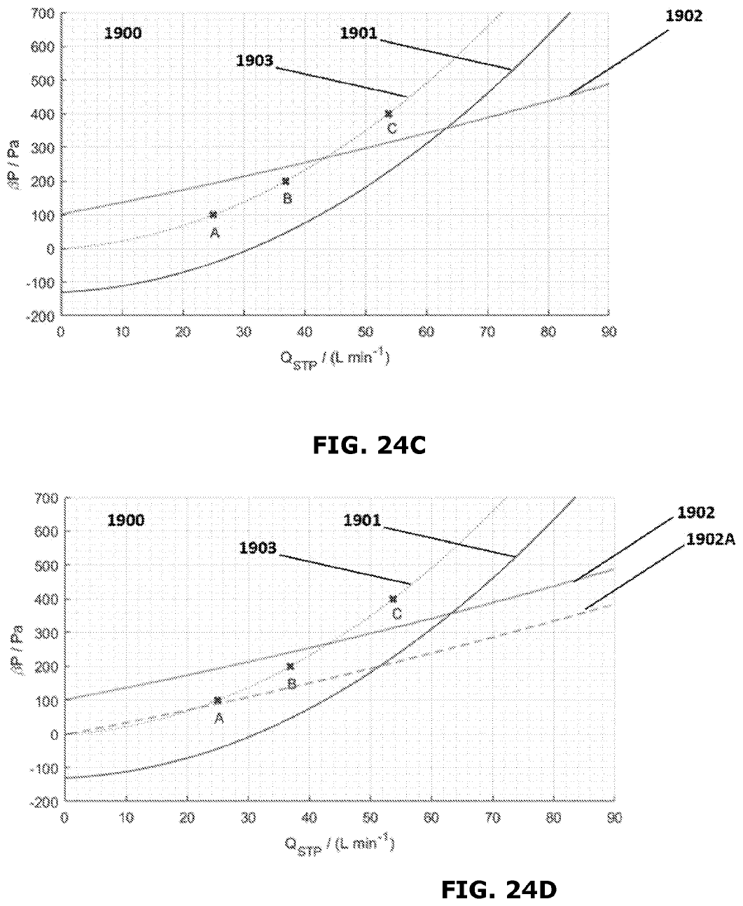

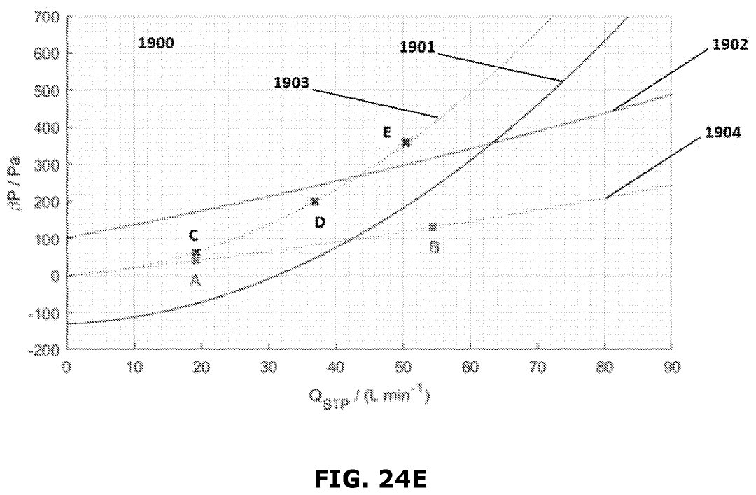

Referring to FIG. 24A, a pressure vs flow rate plot 1900 is shown, to demonstrate the nature of the leak thresholds used in the leak detection algorithm. As previously explained, the leak being detected in this embodiment relates to the humidification chamber being partially or completely removed or disconnected from the flow path of the apparatus, and the leak thresholds are derived based on the pressure versus flow rate characteristic associated with the flow of gases in the apparatus operating normally with the chamber on, and the apparatus operating with a leak with the chamber off.

An example Pₗₑₐₖ threshold function is depicted as a threshold curve or line 1901 on pressure vs flow rate graph at 1900. An example P_{maybe} threshold function is depicted as a threshold curve or line 1902 on pressure vs flow rate graph at 1900.

The function or curve or data represented by 1903 is the sensed pressure characteristic of the flow of gases against the operational flow rate range, for a normally operating apparatus with chamber on. The function or curve or data represented by 1904 is the sensed pressure characteristic of the flow of gases against the operational flow rate range, for an apparatus operating with a leak due to the chamber being off. As shown in the lower flow rate region highlighted at 1905, the pressure vs flow rate curves 1903 and 1904 for normal and leaky operation converge in this region, thereby making it difficult to discriminate a genuine or definite chamber off leak at lower flow rates. The leak detection algorithm of this disclosure provides a means of addressing this issue, by incrementally increasing the motor speed to provide clearer discrimination between the curves for definitively determining a leak (e.g. chamber off) or no leak (e.g. chamber on) condition.

By way of further explanation, referring to FIG. 24B, the definite leak Pₗₑₐₖ threshold function, curve or line 1901 and possible leak P_{maybe} threshold function, curve or line 1902 are shown in isolation on the pressure versus flow rate graph. The region indicated generally at 1906 is a ‘maybe region’ or ‘possible leak region’. As shown, the possible leak region 1906 is a region at the lower end of the flow rate range, above the Pₗₑₐₖ threshold 1901 and below the P_{maybe} threshold 1902. By way of further explanation, measured or sensed pressure values of the flow of gases that fall in this possible leak region 1906 for the associated measured flow rates are considered by the first-stage evaluation 802 of the leak detection algorithm to be possible leaks. These possible leak scenarios are then progressed to the second stage (803-805) of the leak detection algorithm until the condition is resolved as a definite leak condition or no leak condition. By way of example, the second-stage evaluation repeats at successive increased motor speeds until the measured pressure and measured flow rate combination fall outside the possible leak region 1906, such that the possible leak condition is resolved as either a definite leak or no leak.

Referring to FIGS. 24C and 24D, graphical examples of the leak detection algorithm in operation and determining normal operation, i.e. no leak, is shown. In particular, the data points A-C represent the successive sensed pressure and flow rate pairs evaluated in the leak detection algorithm. Data point A is the operating condition of the respiratory apparatus in normal operation. The first-stage evaluation of the leak detection algorithm determines this is in the possible leak region 1906, and advances to the second-stage evaluation by increasing the motor speed of the flow generator of the apparatus. Data point B represents the sensed pressure and flow rate at the higher motor speed. Data point B is still in the possible leak region 1906, so the second-stage evaluation repeats by increasing the motor speed again. Data point C represents the sensed pressure and flow rate at the further increased motor speed and this is outside the possible leak region 1906, i.e. it is above both the Pₗₑₐₖ threshold 1901 and the P_{maybe} threshold 1902. As such, the leak detection algorithm resolves the possible leak as a no leak condition, and reverts to back to normal flow control at the original set flow rate, e.g. reverting back to operating at data point A.

As discussed above, in some embodiments, the leak detection algorithm optionally comprises a threshold adjustment process 807 that executes in scenarios where possible leaks are resolved as a no leak condition. Referring to FIG. 24D, the application and result of this threshold adjustment process 807 is shown. In particular, the threshold adjustment process 807 is configured to adjust the possible leak threshold to avoid the leak detection algorithm progressing in an infinite loop upon return to normal flow control. As shown, the threshold adjustment process 807 modifies or adjusts the P_{maybe} threshold 1902 such that the original data point A is no longer considered a possible leak. In particular, the P_{maybe} threshold 1902 is adjusted or modified or translated downward to an updated or new P_{maybe} threshold 1902A. This adjusted (lower) new P_{maybe} threshold 1902A effectively reduces the possible leak region 1906, and as such data point A is no longer considered by the first-stage evaluation as a possible leak.

Referring to FIG. 24E, a graphical example of the leak detection algorithm in operation and determining a leak, is shown. In this scenario, the measured pressure and flow rate combination during normal operation at data point A is considered a possible leak, as it is located in the possible leak region 1906. The first-stage evaluation identifies the possible leak condition, and progresses to the second-stage evaluation by increasing the motor speed. In this example, the motor speed is increased the new measured pressure and flow rate at the higher motor speed represented at data point B is evaluated by the second-stage evaluation. As data point B falls below the definite leak threshold (Pₗₑₐₖ threshold 1901), the possible leak is confirmed as a definite leak, and the leak detection algorithm triggers an alarm as per stage 806 previously described. Once the leak is resolved (e.g. the chamber is reconnected or installed into the flow path of the respiratory apparatus), the leak detection algorithm allows the respiratory apparatus to revert back to normal flow control at the original set flow rate. As shown, once the leak is resolved and normal flow control resumes, the leak detection algorithm reverts to the first-stage leak evaluation. Data point C represents the new measured pressure and flow rate at the original set flow rate, after the leak is resolved. The leak detection algorithm then progresses as per the process described in regard to FIGS. 24C and 24D, i.e. data point C is considered a possible leak in the first-stage evaluation. The motor speed is increased to data point D in the second-stage evaluation, which is still a possible leak condition. The motor speed is increased against to data point E in the second-stage evaluation and which is resolved as a no leak condition, i.e. normal operation. As per the description above, the P_{maybe} threshold 1902 is then adjusted and the flow control returns to normal and the leak detection algorithm reverts to the first-stage evaluation to continue processing incoming data as the apparatus continues to operate.

As discussed above, the Pₗₑₐₖ threshold 1901 and the P_{maybe} threshold 1902 curves or lines may be derived from functions, in some embodiments. By way of example only, and for one particular respiratory apparatus configuration, the equations or functions may be in the form of the following:

$${P}_{l e a k} = \frac{1}{β} ( {k}_{1} {Q}_{stpd , filt}^{2} + {k}_{2} {Q}_{stpd , filt} + {k}_{3} \qquad ( 1 )$$

$${P}_{maybe} = \frac{1}{β} ( {k}_{4} {Q}_{stpd , filt}^{2} + {k}_{5} {Q}_{stpd , filt} + {k}_{ó} ) \qquad ( 2 )$$

Where Q_{stpd,filt} is the filtered STPD flow rate, k₁-k₆ are constants that are specific to the apparatus and/or flow path characteristics and/or the mode of operation, and the value β is a function based at least partly on the sensed ambient pressure the apparatus is operating in and the sensed temperature of the flow of gases (e.g. the temperature of the flow of gases at or near the outlet of the humidification chamber or some other suitable location in the flow path). In this example, equations (1) and (2) may be quadratic functions.

In some embodiments, one or more of the constants k₁-k₆ defining the leak threshold functions may depend on the mode of operation of the respiratory apparatus and/or specific types of leak being detected. By way of example, in one embodiment, the constants k₁-k₆ used for the leak detection algorithm in therapy mode and drying mode may be the same, such that the thresholds are the same in both these modes. However, different constants k₁-k₆ may be employed to define different threshold curves when the leak detection algorithm is operating in disinfection mode, as the flow path characteristics are different with the disinfection kit assembly installed.

Equations or functions (1) and (2) are provided by way of example only. It will be appreciated that the leak thresholds or functions may be customised based on a number of factors or characteristics relating to the specific respiratory apparatus and/or its mode(s) of operation, for example.

It will be appreciated that the leak detection algorithm may employ or apply different leak threshold functions or curves, depending on the nature or type of leak being detected.

### Drying Mode

As discussed above, the leak detection algorithm may be configured to also operate in one or more non-therapy modes. On such non-therapy mode is ‘drying mode’. Drying mode is typically performed at the end of a therapy session with the respiratory apparatus to dry out the flow path and/or components in the flow path.

Drying mode is typically initiated after the user or patient has removed the patient interface (e.g. nasal cannula), i.e. at some stage after the therapy session has ended or if the therapy session has been otherwise terminated via an ‘off’ or ‘stop’ input to a user interface (e.g. touchscreen interface) of the respiratory apparatus. Drying mode causes the flow generator to operate at a constant flow rate, e.g. between 15 L/min to 30 L/min, preferably 25 L/min, or some other suitable flow rate. Alternatively, the flow generator may be controlled to a specific or configurable constant motor speed. Typically, during drying mode, the heater plate of the humidifier is switched off or reduced to a low power output. Typically, the drying mode operates for a configurable or pre-set time period, in order to dry out the flow path and/or flow path components.

The leak detection algorithm 800 may operate in the drying mode in accordance with the configuration described above with respect to FIGS. 23-24E. The main difference is that the respiratory apparatus is operating at a pre-set drying mode flow rate or motor speed in accordance with the drying mode settings, rather than a set therapy flow rate that is specific to a user or patient during a therapy session in therapy mode. As such, the leak detection algorithm in drying mode tends to start or operate the first-stage evaluation at the same pre-set drying mode flow rate. As with the therapy mode operation, the leak detection algorithm is configured to detect a leak during drying mode, such as a ‘chamber off’ condition (e.g. chamber removed or disconnected from the flow path) and raises an alarm if such a condition is detected.

As with therapy mode, in this example embodiment, the humidification chamber is also an essential part of the flow path of the apparatus for drying mode. If the humidification chamber is removed or disconnected from the apparatus, then the flow path is broken and the drying mode won't be able to dry the patient circuit (e.g. patient conduit and/or patient interface) as the flow of gases will leak into the ambient environment rather than flow into the patient circuit. Therefore, the leak detection algorithm is configured to determine the chamber connection status, and alert the user to a fault in the drying mode operation if the chamber is off or disconnected.

In this example, the pre-set drying mode flow rate tends to operate in a lower flow rate region where it is difficult to discriminate between a leak condition (e.g. chamber is off) and normal operation for the reasons previously explained. Hence the leak detection algorithm is also able to assist in robustly determining a leak condition in drying mode.

In this example embodiment, the flow path characteristics in the therapy mode and drying mode are similar, and therefore the same leak threshold functions may be employed in the leak detection algorithm.

### Heater Plate Check to Confirm a Chamber Off Leak Condition

In some embodiments, the leak detection algorithm may be augmented or modified to optionally include a heater plate check to confirm or determine if the humidification chamber has been removed from the flow path and/or humidification compartment of the respiratory apparatus.

In a first configuration of the heater plate check, the leak detection algorithm is configured to energise the heater plate of the humidifier to a set power level (e.g. 100% power or 50% power) for a set period of time (e.g. apply a power process or procedure to the heater plate). The algorithm monitors the temperature increase of the heater plate based on sensed temperature data or signals generated by a temperature sensor or sensors configured to sense the temperature of the heater plate (e.g. a temperature sensor in or associated with the heater plate). The algorithm may be configured to calculate a rate of change of temperature based on the temperature sensor data as the heater plate is energised to the set power level (i.e. the heating rate of the heater plate). The rate of change of temperature is then compared to a rate of change threshold. If the rate of change of temperature is greater than the rate of change threshold, then a chamber off (e.g. leak condition) is confirmed or indicated. Alternatively, the algorithm may calculate or determine the time taken for the temperature of the heater plate to exceed a set temperature threshold. The time taken is then compared to a time threshold. If the time taken is below the time threshold, then a chamber off (e.g. leak condition) is confirmed or indicated.

In a second configuration of the heater plate check, the leak detection algorithm is configured to energise or heat the heater plate of the humidifier to a pre-set temperature (e.g. apply a temperature process or procedure to the heater plate). Once at the pre-set temperature, the power to the heater plate is then switched off, and the cooling rate of the heater plate is monitored based on the temperature data or signals from the heater plate temperature sensor or sensors. The cooling rate is less than a cooling rate threshold, then a chamber off (e.g. leak condition) is confirmed or indicated. Alternatively, the algorithm may calculate or determine the time taken for the temperature of the heater plate to reduce or cool back to a pre-set lower temperature, e.g. ambient temperature or some other suitable lower temperature. The time taken is then compared to a time threshold. If the time taken is above the time threshold, then a chamber off (e.g. leak condition) is confirmed or indicated.

Both of the first and second configuration heater plate checks are based on the missing thermal mass of the water in the humidification chamber when the chamber is removed from the humidification unit. In particular, without the water-filled humidification chamber being in thermal contact with the heater plate, the heater plate heats up more quickly or cools more slowly.

Either or both of the above first and second heater plate checks may be implemented or executed by the controller of the respiratory apparatus as part of the leak detection algorithm or as a separate heater plate check algorithm. The output of the heater plate checks may be an output indicator or data indicative of a leak or no leak condition (e.g. chamber on or chamber off). Examples are provided below of augmenting or modifying the leak detection algorithm with a heater plate check are described below.

In one first example embodiment, the leak detection algorithms 700, 800 described above can be optionally augmented with either of the first or second configuration heater plate checks, as part of the leak detection algorithm. Referring to FIG. 23, in this first example embodiment, the heater plate check 808 is executed as an additional or supplementary process or leak confirmation stage, before entering the leak detected state (704, 806). In particular, if the leak detection process determines a definite leak after either the first-stage (702, 802) or second-stage (707, 803-805) leak evaluations, a heater plate check or checks 808 may be initiated in accordance with either or both of the first or second configurations described above. If the heater plate check or checks confirm the leak condition (e.g. chamber off), then the leak detection algorithm proceeds to the leak detected state (704, 806) and triggers the one or more alarm actions. In this example, the heater plate check or checks 808 further confirm the leak condition, before the algorithm moves to the leak detected stage in which alarm actions are initiated.

In a second example embodiment, the leak detection algorithms 700, 800 described above may be modified such that the second-stage leak evaluation (706, 803-805) is replaced with one or more heater plate checks, rather than stepping through increased motor speeds and re-checking the sensed pressure against the leak thresholds until a possible leak condition is resolved as either a leak or no leak condition. In this second example embodiment, the leak detection algorithms 700, 800 are modified such that the one or more of the heater plate checks is initiated after a possible leak condition is detected in the first-stage leak evaluation (702, 802). The one or more heater plate checks are implemented following the possible leak condition being detected in order to resolve the possible leak condition as a definite leak condition or a no leak condition. If the heater plate check or checks confirm the possible leak condition as a definite leak (e.g. chamber is off), then the algorithm progresses to the leak detected state (704, 806). If the heater plate check or checks resolve the possible leak is a no leak condition (e.g. chamber is connected), then the algorithm progresses in accordance with the previously described steps that apply to exiting the second-stage leak evaluation after resolving that no leak is detected. In particular, the leak thresholds may be updated (712, 807), and the algorithm reverts to normal operation or flow control, and reverts back to the first-stage leak evaluation stage to further process the next incoming data streams as the apparatus continues to operate. In this second example embodiment, there is no stepped increases in motor speed to resolve a possible leak condition in the second-stage leak evaluation, rather a heater plate check or checks are implemented to resolve the possible leak as a definite leak condition or no leak condition.

The above first and second example embodiments of the heater plate checks being used in modified leak detection algorithms may be employed when the respiratory apparatus is being operated in therapy mode or drying mode.

### 3.3 Detailed Second Example of a Leak Detection Process-Disinfection Mode

### Disinfection Kit or Assembly

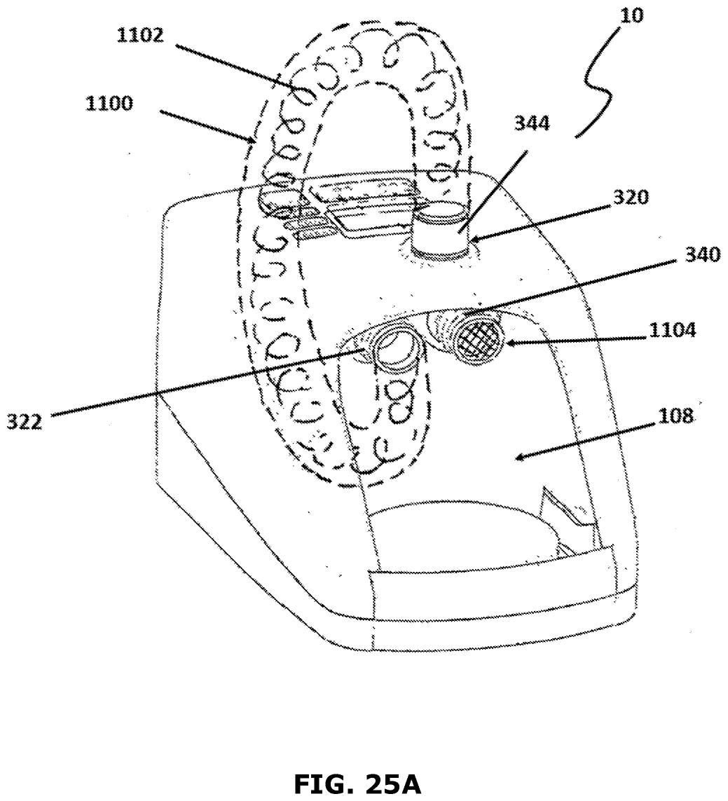

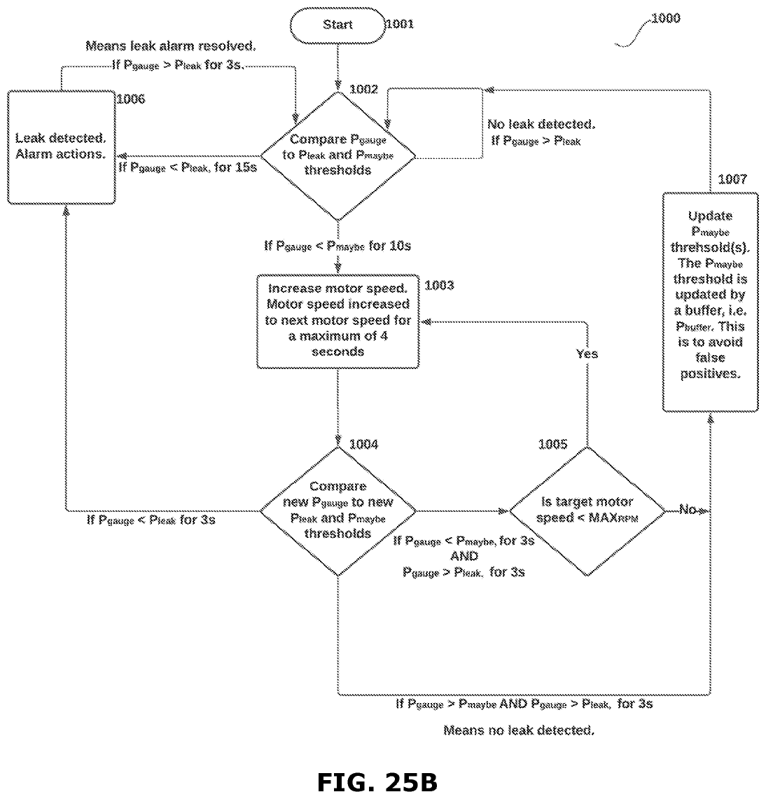

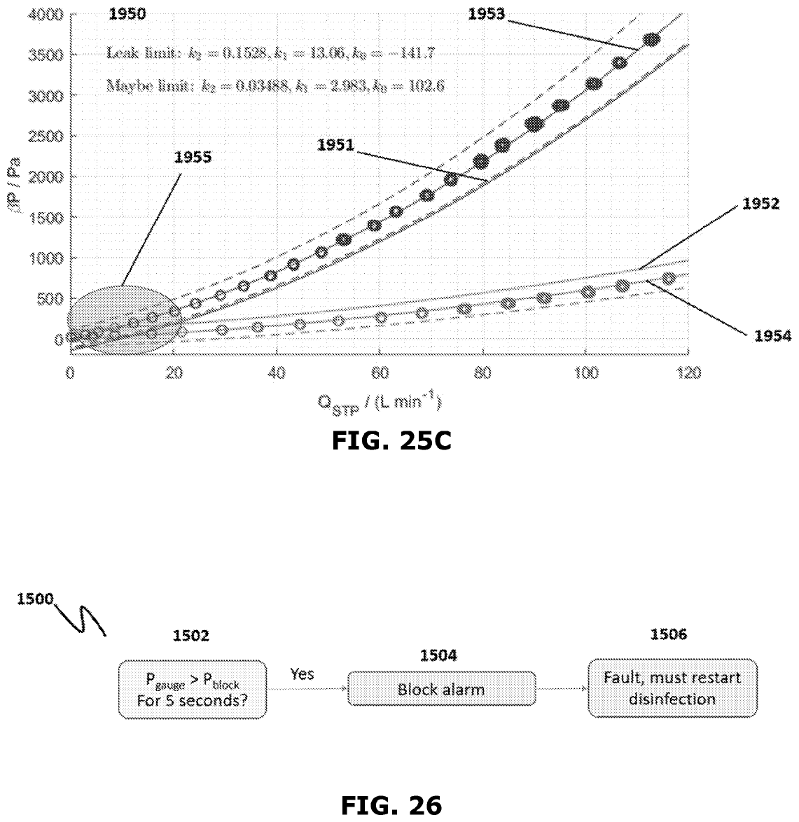

The leak detection algorithm can be configured to operate during other non-therapy modes of the respiratory apparatus. One such other non-therapy mode is a disinfection mode. In brief the respiratory apparatus may have an operable disinfection mode that is configured to disinfect one or more components in the flow path following a therapy session. An example embodiment of the leak detection algorithm operating in the disinfection mode will now be explained with reference to FIGS. 25A-25C. FIG. 25A is a simplified schematic drawing of a respiratory apparatus of the type described with respect to FIGS. 2-21, and like numerals represent like or similar components, for the purpose of the explanation.

In one example embodiment, the disinfection mode of the respiratory apparatus may operate in generally in accordance with the principles, description and/or configuration described in International PCT Application Publication WO2007/069922 filed 15 Dec. 2006, which is hereby incorporated by reference in its entirety.

Referring to FIG. 25A, in this example embodiment, the disinfection mode of the respiratory apparatus 10 is operable to disinfect the elbow conduit 320 (e.g. ‘removable elbow’) in the flow path of the respiratory apparatus. Prior to initiating the disinfection mode, the humidification chamber 300 is removed from the flow path and humidification chamber bay or compartment 108 of the respiratory apparatus. A disinfection kit or assembly is then installed into the flow path. The disinfection kit or assembly comprises disinfection tube or conduit 1100 and a filter or filter assembly 1104. The disinfection tube 1100 can comprise an integral heating element 1102. A first end of the disinfection tube 1100 is connected to the gases outlet port 322 and a second end of the disinfection tube is connected to the patient outlet port 344 or the elbow conduit 320. The filter 1104 or filter assembly or component is inserted into or over the gases inlet port 340 of the elbow conduit. In one configuration, the filter 1104 may comprise a filter cap or cap with a filter, with the filter cap or cap engaging with, in, or over the gases inlet port 340. In one example, the filter is received and retained in the gases inlet port 340 via friction fit. The configuration of the disinfection tube and filter may be the same or similar to that described in WO2007/069922. The filter 1104 is configured to filter 1104 the flow of gases driven through and exiting the elbow conduit 320. For example, the filter 1104 is configured to filter out pathogens and/or particulate matter in the flow of gases generated during the disinfection mode.

In an alternative configuration, the disinfection kit or assembly comprises an Ozone module that may be coupled to the respiratory apparatus. The leak detection method may be configured to detect a dislodgement or misconnection of the ozone module that is part of the disinfection kit. The ozone module may be configured to pump ozone gas through the flow path of the respiratory apparatus in order to disinfect the flow path. The ozone module may be required to be used for a set period of time. The described leak detection method is configured to detect removal of the disinfection module (i.e. Ozone module) prior to the required time, and thereby cause an appropriate alarm.

### Disinfection Mode

The disinfection mode may be selected by the controller of the respiratory apparatus automatically at the end of a therapy session and/or upon detection of the disinfection tube being connected into the flow path, or may be manually selected by a user via the control interface.

Once the disinfection kit or assembly comprising the disinfection tube 1100 and filter 1104 are installed into the flow path, the disinfection mode may be initiated. During the disinfection mode the respiratory apparatus is operated to generate a high temperature flow of gases through the disinfection tube 1100, elbow conduit 320, and out the filter 1104 to atmosphere or the ambient environment for a predetermined time period to disinfect the elbow conduit 320.

In this example embodiment, during disinfection mode, the flow generator is operated at a constant preconfigured or pre-set flow rate. In one configuration of the disinfection mode there is an initial flush stage and a disinfection stage. The initial stage is configured to generate a higher flow rate of gases for a predetermined or minimum time period to flush out any mobile condensate in the elbow conduit 320. Once the flush stage is completed, the disinfection stage commences and operates at a lower flow rate and high temperature to disinfect the elbow conduit 320 for a predetermined or minimum time period.

In one example configuration, the initial flush stage may comprise operating the flow generator at a flow rate of about 30 L/min for between 20 seconds and 5 minutes, or preferably between 30 seconds and 2 minutes. Once the initial flush stage is complete, the disinfection stage or cycle commences which delivers a high temperature flow of gases at a flow rate of about 11 L/min, in this example. In some embodiments, the disinfection tube 1100 is operated to heat the flow of gases generated by the flow generator to a high temperature, e.g. above 70° C., preferably above 85° C., and more preferably between 85° C. and 95° C., during the disinfection stage or cycle.

During the disinfection mode, one or more leaks in the flow path may occur. In one example, the filter cap of the filter 1104 of the disinfection kit or assembly may become dislodged or could be loosened from the gases inlet port 340 of the elbow conduit 320, e.g. due to the apparatus being bumped. In another example, either or both ends of the disinfection tube 1100 may become disconnected or loosened from their respective ports 322 and/or 344, causing a leak.

### Leak Detection Algorithm During Disinfection Mode

The leak detection algorithm of this disclosure may be configured to operate during the disinfection mode to detect one or more types of leaks in the flow path, e.g. removal or disconnection of the filter 1104 or component of the filter assembly (e.g. filter cap) from the elbow conduit 320, and/or a disconnection or loose connection of the disinfection tube to the ports 322 and/or 344.

In one example configuration, the leak detection algorithm may be configured with leak thresholds that are tuned to detect whether the filter 1104 or a filter component is present (i.e. no leak), or removed or not connected to the elbow conduit (i.e. leak condition). If the algorithm detects a leak condition indicating that the filter 1104 or a filter component is not connected, an alarm is raised or triggered. The filter 1104 is an important component to safe operation of the disinfection mode. The disinfection mode causes very hot gases to exit the elbow conduit 320 at port 340. The filter 1104 cools the gases and captures any particulate matter as the flow of gases exit the elbow conduit 320.

As described above, the disinfection stage of the disinfection mode tends to operate a low constant flow rate and in this region it is difficult to discriminate between a leak condition and normal operation, for the reasons previously described. Hence the leak detection algorithm is also able to assist in robustly determining a leak condition (e.g. filter or filter cap off or not connected) in this disinfection mode.

Referring to FIG. 25B, the operation of the leak detection algorithm 1000 for disinfection mode will be described in further detail, by way of example only. The leak detection algorithm 1000 operates in a similar manner to the leak detection algorithm 800 of FIG. 23 and in accordance with the principles of the overview example algorithm 700 described with respect to FIG. 22. In this example, the leak detection algorithm 1000 is configured to detect leaks or leak conditions caused by the removal or dislodgement of the filter 1104 or a filter component, or disconnection or leaks in the connection of the disinfection tube 1100 if the disinfection kit or assembly.

In this example embodiment, the leak detection algorithm 1000 starts after the initial flush stage or cycle of the disinfection mode has completed and the disinfection stage or cycle has commenced with the apparatus generating a high temperature flow of gases at a ‘disinfection flow rate’ (e.g. a constant flow rate of about 11 L/min in this example) through the elbow conduit 320. The leak detection algorithm may operate continuously during the disinfection cycle.

### First-Stage Leak Evaluation

In this embodiment, the leak detection algorithm 1000 starts 1001 by proceeding to the first-stage leak evaluation at step 1002. In this first-stage leak evaluation 1002, the gauge pressure variable (representing the sensed gauge pressure of the flow of gases) is compared to definite and possible leak thresholds to determine the leak state of the apparatus, e.g. leak detected, possible leak, or no leak. In this embodiment, one or each condition may be considered as being satisfied or detected based on respective evaluation criteria being satisfied, as will be explained below. In this embodiment, the various evaluations based on comparing the gauge pressure to the one or more threshold(s), may occur concurrently or in parallel. In other configurations, the evaluations of which leak condition is satisfied may be configured to occur sequentially, or in a particular conditional order.

While operating in the first-stage leak evaluation 1002, the apparatus can be considered as being in normal disinfection operation with no leak detected, i.e. in the no leak state (e.g. filter 1104 or filter cap connected). In this state or stage, the respiratory apparatus continues with normal disinfection operation in accordance with the disinfection flow rate.

In this embodiment, the algorithm retrieves and/or receives input data representing the measured or sensed flow rate of the flow of gases, the measured or sensed gauge pressure variable, and the leak thresholds, for evaluation. The input data may be retrieved in accordance with a particular sampling frequency or continuously as the data becomes available from the sensors and/or main controller and/or memory of the apparatus. In one configuration, the input data may be a moving average based on a moving window of data. The window of data may be determined based on a configuration time period or number of data samples, for example. In one example, the input data may be a 10 second moving average of the sensed flow rate, sensed gauge pressure, and leak thresholds. In another configuration, the input data used by the leak detection algorithm may be the latest instantaneous data for the sensed flow rate, sensed gauge pressure, and leak thresholds.

In this embodiment, at step 1002, one of the evaluations undertaken by the algorithm is to determine a definite leak condition. In this embodiment, to determine a definite leak condition, the algorithm compares the gauge pressure variable to a definite leak threshold Pₗₑₐₖ (which is a pressure threshold in this embodiment) representing a definite leak condition. If the gauge pressure variable P_{gauge} is less than the definite leak threshold Pₗₑₐₖ for a minimum evaluation time, then a leak condition is satisfied and the algorithm raises or generates a leak alarm at 1006. In one configuration, the gauge pressure variable P_{gauge} must be below the definite leak threshold Pₗₑₐₖ for a minimum evaluation time period of 15 seconds, although it will be appreciated this time period may be adjusted or varied in other embodiments.

If the leak detection condition is satisfied based on the evaluation at step 1002, the algorithm moves to the leak alarm or leak detected state or stage at 1006. In the leak detected state 1006, the algorithm may trigger one or more alarm actions, such as generating an audible, visual or tactile leak detection alarm, indicating the filter of the disinfection kit or assembly is off or disconnected from the elbow conduit 320, and/or controlling or halting the flow rate or motor speed, or other control actions.

In this embodiment, the algorithm 1000 in the leak detected state 1006 may be configured to execute one or more alarm actions. In this example embodiment, the algorithm 1000 may execute any one or more of the following example alarm actions:
freezing the current target motor speed at which the leak was detected, such that the motor speed does not change from its present value while in the leak alarm state 1006.
raise or trigger an audible, visual and/or tactile alarm or leak fault notification to the user (e.g. message or notification on display of the respiratory apparatus and/or audible alarm via speaker or audio device of the respiratory apparatus). For example, the user may be prompted to reconnect the filter 1104 or filter cap onto the elbow 320, and restart the apparatus and/or disinfection mode.

Once in the leak detected state 1006, the algorithm 1000 may also be configured to determine or evaluate whether the leak alarm has been resolved, for example whether the filter has been reconnected or installed back into elbow conduit 320 of the apparatus. In this embodiment, the algorithm at 1006 continually evaluates or determines whether the leak has been resolved by comparing the pressure variable P_{gauge} to the definite leak threshold Pₗₑₐₖ or another alternative threshold specific to confirm leak resolution. In this example embodiment, if the gauge pressure variable P_{gauge} is over the Pₗₑₐₖ threshold for a minimum evaluation time period, which in this embodiment is 3 seconds but this could be adjusted in other embodiments, the leak alarm is considered as resolved. If the leak alarm is resolved, the algorithm returns to the first-stage leak evaluation 1002 and normal disinfection mode operation may recommence or restart in accordance with the set disinfection flow rate. If the gauge pressure variable does not exceed the Pₗₑₐₖ threshold for more than the minimum evaluation time period, then the algorithm remains in the leak detected state 1006 and continues to check for resolution of the leak alarm.

In this embodiment, at step 1002, one of the other evaluations undertaken by the algorithm is to determine a possible leak condition. In this embodiment, to determine a possible leak condition, the algorithm compares the gauge pressure variable P_{gauge} to a possible leak threshold P_{maybe} (which is a pressure threshold in this embodiment) representing a possible or potential leak condition. In this embodiment, if the gauge pressure variable P gauge is below the possible leak threshold P_{maybe} for a minimum evaluation time period, a possible leak condition is satisfied and the algorithm moves to the second-stage leak evaluation (which commences at 1003) to resolve the identified possible leak as a definite leak or no leak. In this embodiment, the minimum evaluation time period is 10 seconds, but this may be varied in different embodiments.

If neither of the definite leak or possible leak conditions are met based on the evaluations described above, the algorithm considers no leak is detected, and remains in the first-stage leak evaluation 1002 in normal disinfection mode operation, i.e. no leak condition remains satisfied (e.g. the disinfection filter is in the elbow conduit), and the apparatus continues with normal disinfection cycle.

### Second-Stage Leak Evaluation

If the algorithm detects a possible leak condition at 1002, the algorithm moves to the second-stage leak evaluation, commencing at step 1003. In this embodiment, before entering the second-stage leak evaluation to resolve the possible leak as a definite leak or no leak, the algorithm 1000 is configured to store the measured flow rate (e.g. sensed by a flow rate sensor in the flow path or other sensing configuration) and the sensed pressure variable occurring before entering the second-stage leak evaluation at step 1003. In one configuration, the stored values of the measured flow rate and stored sensed pressure variable may represent or be extracted from moving averages of these measured or sensed variables, as explained above. For example, in one configuration, the algorithm either receives during operation or calculates during operation during moving averages of the measured flow rate and sensed pressure variable, from which it can extract and store values prior to entering the second-stage leak evaluation. By way of example, the stored values of measured flow rate and sensed pressure variable can be used in later algorithm steps involving updating and/or adjusting one or more of the leak threshold limits.

In this embodiment, the second-stage leak evaluation comprises increasing the current target motor speed by a predetermined increment or to a first or next pre-set higher motor speed at 1003. In this embodiment, the algorithm is configured to increase and lock the motor speed at the first higher motor speed for a predetermined or maximum time period. In one configuration, the time period may be 4 seconds, but this may be varied in alternative embodiments. As will be explained below, during the or each iteration to a next higher motor speed, the higher motor speed is maintained for the configured time period (e.g. 4 seconds in this example) while the second-stage leak evaluations 1004 are undertaken to attempt to resolve the possible leak as a definite leak condition or a no leak condition.

Once at the higher motor speed after the increase at 1003, the algorithm 1000 commences with one or more second-stage leak evaluations at 1004. For example, the updated or new sensed pressure variable P_{gauge} at the higher motor speed is compared to new or updated definite Pₗₑₐₖ and possible P_{maybe} thresholds to determine whether the possible leak condition can be resolved as a definite leak condition or a no leak condition. Each condition may again have its own evaluation criteria to satisfy, based on comparing the sensed gauge pressure variable P_{gauge} to one or more of the leak thresholds, as will be described below.

In this embodiment, at step 1004, one of the evaluations undertaken by the algorithm is to resolve the possible leak condition as a definite leak condition. In this embodiment, to determine a possible leak condition, the algorithm compares the updated sensed pressure variable P_{gauge} to an updated definite leak threshold Pₗₑₐₖ. In this embodiment, the new Pₗₑₐₖ threshold is updated or determined based at least partly on the new higher motor speed and/or flow rate of the flow of gases generated at the first higher motor speed. If the gauge pressure variable P_{gauge} is below the Pₗₑₐₖ threshold for a minimum evaluation time period (e.g. 3 seconds or another configurable time period), the possible leak condition is confirmed or resolved as a definite leak condition, and the algorithm progresses to the leak detected state 1006 and generates a leak alarm or initiates one or more alarm actions as described previously.

In this embodiment, one of the other evaluations undertaken by the algorithm is to resolve the possible leak condition as a no leak condition. In this embodiment, to determine a no leak condition, the algorithm compares the updated sensed pressure variable P_{gauge} to updated possible leak threshold P_{maybe} and updated definite leak threshold Pₗₑₐₖ. As explained above, the updated thresholds may be extracted or determined based at least partly on the new higher motor speed and/or flow rate of the flow of gases generated at the first higher motor speed. If the updated sensed gauge pressure variable P_{gauge} is above both the P_{maybe} threshold and above the Pₗₑₐₖ threshold for a minimum evaluation time (e.g. 3 seconds or another configurable time period), then the possible leak is resolved as no leak detected, and the algorithm reverts to the first-stage evaluation 1002 and normal operation and/or flow control.

In this embodiment, upon exiting the second-stage leak evaluation at 1004 (after resolving the possible leak is not a leak) and prior to reverting to step 1002 and normal operation 1001, the algorithm can optionally execute a threshold adjustment process at 1007. In this embodiment, the threshold adjustment process 1007 can be configured to adjust or reduce the P_{maybe} threshold to prevent the algorithm endlessly looping in the same scenario in future checks. In one configuration, the P_{maybe} threshold may be adjusted based on an adjustment or correction function 1007 having one or more variables. By way of example, the variables of the adjustment or correction function may comprise the measured flow rate and sensed pressure variable values stored prior to entering the second-stage leak evaluation at step 1003, as previously discussed. Additionally, the adjustment or correction function may include one or more further variables or constants. In one configuration, the adjustment or correction function may have a constant variable that is dependent on the stored measured flow rate, such that the magnitude of adjustment may vary depending on the stored measured flow rate. In one configuration, the constant variable may be configured to cause the adjustment or correction function to make smaller adjustments to the P_{maybe} threshold at lower flow rates or lower flow rate ranges.

In one embodiment, the threshold adjustment function 1007 may comprise updating the P_{maybe} threshold based at least partly on a buffer value P_{buffer}. In one configuration, the P_{buffer} value may be at least partly dependent on the measured flow rate stored after exiting the first-stage 1002. In another configuration, the P_{buffer} value may be a constant. In one configuration, the P_{maybe} threshold is extracted from a threshold function that is dependent on the measured flow rate and one or more constants. In this example configuration, the threshold adjustment function is configured to adjust the one or more constants of the threshold function based at least partly on the P_{buffer} value. In another example, the threshold adjustment function is configured to adjust the one or more constants of the threshold function based at least partly on the P_{buffer} value, and the P_{maybe} threshold value and measured pressure value stored after existing the first-stage 1002.

In some configurations of the adjustment or correction function 1007, the P_{maybe} threshold at the discrete measured flow rate sensed prior to entering the second-stage leak evaluation at 1003 may be reduced or decremented by a calculated or predetermined amount. In other configurations, the entire P_{maybe} threshold function or threshold curve may be reduced or decremented by a calculated or predetermined amount across the entire operating flow rate range.

Reverting to the second-stage leak evaluations at 1004, if neither of the definite leak or no leak conditions are satisfied based on the evaluations described above, the possible leak condition is considered as unresolved. In this embodiment, if the possible leak condition is unresolved for a predetermined time period based on a timer (e.g. 4 seconds in this example, but could be another configurable time period), then the second-stage leak evaluation process repeats, but at a further higher motor speed.

In this embodiment, if the timer expires and the possible leak condition is unresolved, the algorithm exits 1004 and initiates a motor speed check 1005. In this embodiment, the motor speed check 1005 comprises comparing the current target motor speed against a pre-set or configurable motor speed threshold (MAX_{RPM}), before repeating or looping back to the start 1003 of the second-stage leak evaluation.

In this embodiment, if the current target motor speed is equal to or above the motor speed threshold MAX_{RPM} at the motor speed check 1005, the algorithm exits the second-stage leak evaluation and resolves the possible leak as a no leak condition, and reverts to the first-stage 1002 and normal operation via the threshold adjustment process 1007 previously explained. The motor speed check 1005 provides an additional exit condition for the algorithm, and prevents the algorithm from endlessly looping in the second-stage and/or from increasing the motor speed beyond safety or component thresholds.

If the current target motor speed is below the motor speed threshold MAX_{RPM} at the motor speed check 1005, the algorithm loops back to step 1003, and again increases the first higher motor speed to a second or next higher motor speed, and repeats the evaluations at 1004 again, in an attempt to resolve the possible leak as a definite leak or no leak condition.

As shown, the second-stage comprises continuously or repeatedly incrementing the motor speed by a predetermined amount or to a next predetermined higher motor speed, and then undertaking evaluations at 1004 until the leak condition is satisfied, the no leak condition is satisfied, or the motor speed check fails at 1005. As will be appreciated, depending on the evaluations, the second-stage of the algorithm may complete once, twice or multiple times, prior to exiting back to 1002 (no leak) or 1006 (leak detected).

At step 1003, it will be appreciated that the increments in motor speed may be pre-set or configurable increments or may increment to the next highest motor speed in a pre-set or configurable series or array of preselected discrete higher motor speeds. By way of example only, in one configuration, the motor speed increase at step 1006 is based on incrementing the current motor speed to the next highest motor speed in the series or array comprising motor speeds of 5000 rpm, 6750 rpm, 8500 rpm, 10250 rpm, and 12000 rpm, such that the motor speed increases by 1750 rpm each cycle or loop of the second-stage leak evaluation. In this example, there are five possible discrete motor speed stages in which the pressure variable is compared against the P_{maybe} and Pₗₑₐₖ thresholds, but this may be varied to more or less stages as desired

In this embodiment, the leak detection algorithm 1000 repeats once it returns to the first-stage leak evaluation and normal disinfection operation at 1002. In one configuration, the leak detection algorithm continually operates as it receives updated real-time sensed pressure variable data from the pressure sensor(s). In other configurations, the leak detection algorithm may be configured to operate periodically. As previously discussed, in some configurations, upon the apparatus commencing or re-commencing normal disinfection operation, the leak evaluation comparisons against the leak thresholds May be suspended or delayed for a predetermined delay period after specific events. Such events may include, but are not limited to, commencement of a new disinfection cycle, re-starting of the apparatus, resolution of a prior leak alarm at 1006 (e.g. filter of disinfection kit re-installed or re-connected), or otherwise when normal disinfection operation is resumed at 1002, for example after a leak alarm has been resolved or after exiting the second-stage leak evaluation with no leak detected.

### Leak Thresholds for Disinfection Mode

The leak detection algorithm 1000 for disinfection mode operates in a substantially similar manner to the leak detection algorithm 800 in normal therapy mode or drying mode, as described above. However, the flow path of the apparatus 10 or system is different in disinfection mode as the disinfection kit or assembly components are inserted into the flow path, and the humidification chamber and patient circuit are removed or not present. As such, the leak detection algorithm 1000 relies on different leak thresholds that are specific to the modified flow path characteristics caused by the disinfection kit or assembly (e.g. disinfection tube 1100 and filter 1104) being in the flow path.

Referring to FIG. 25C, an example of the different leak thresholds (e.g. leak curves or functions) for the Pₗₑₐₖ and P_{maybe} thresholds are shown and will be described further (and these are may be compared to the different leak thresholds described in regard to FIG. 24A used in the therapy and drying modes).

Referring to FIG. 25C, a pressure vs flow rate plot 1950 is shown (similar to that of FIG. 24A), to demonstrate the nature of the leak thresholds used in the leak detection algorithm for disinfection mode. As previously explained, the leak being detected in this embodiment relates to the filter component of the disinfection kit being partially or completely removed or disconnected from the flow path of the apparatus and/or the disinfection tube being disconnected or loosely connected in a leaky manner, and the leak thresholds are derived based on the pressure versus flow rate characteristic associated with the flow of gases in the apparatus operating normally with the disinfection kit components property installed, and the apparatus operating with a leak with the filter component off and/or disinfection tube removed or loosely connected in a leaky fashion.

An example Pₗₑₐₖ threshold function is depicted as a threshold curve or line 1951 on pressure vs flow rate graph at 1950. An example P_{maybe} threshold function is depicted as a threshold curve or line 1952 on pressure vs flow rate graph at 1950.

The function or curve or data represented by 1953 is the sensed pressure characteristic of the flow of gases against the operational flow rate range, for a normally operating apparatus in disinfection mode (i.e. no leaks in the disinfection kit and/or flow path). The function or curve or data represented by 1954 is the sensed pressure characteristic of the flow of gases against the operational flow rate range, for an apparatus operating in the disinfection mode with a leak due to the disinfection filter off and/or disinfection tube disconnected or loosely connected.

As shown in the lower flow rate region highlighted at 1955, the pressure vs flow rate curves 1953 and 1954 for normal and leaky operation converge in this region, thereby making it difficult to discriminate a genuine or definite filter off or disconnected disinfection tube leak at lower flow rates, such as the disinfection mode flow rate, which in this example is about 11 L/min or any other suitable flow rate for disinfection mode. The leak detection algorithm of this disclosure provides a means of addressing this issue, by incrementally increasing the motor speed to provide clearer discrimination between the curves for definitively determining a leak (e.g. filter off) or no leak (e.g. filter on) condition.

In this example configuration, the Pₗₑₐₖ threshold 1951 and the P_{maybe} threshold 1952 curves or lines may be derived from the same functions or equations (1) and (2) described above in section 3.2 for the therapy and drying mode implementations. As discussed above, one or more of the constants k₁-k₆ in the equations (1) and (2) will be different for the disinfection mode leak curves compared to the leak curves used in the therapy and drying modes. In particular, the one or more constants k₁-k₆ used in the leak threshold functions are specific to the characteristics of the disinfection mode flow path and/or particular types of leaks being detected in the disinfection mode (e.g. filter off and/or loose or disconnected disinfection tube).

### Heater Plate Check to Confirm Disinfection Mode Set-Up

Optionally, in one embodiment, the controller of the apparatus 10 may be configured to implement one or more of the heater plate checks described in section 3.2 above prior to commencing the disinfection mode. In particular, one or more of the heater plate checks may be executed to check that the chamber is disconnected or off, which is a requirement for the disinfection mode to operate and to allow the disinfection kit to be installed into the flow path. If the heater plate checks indicate that the chamber is still on, then the disinfection mode may be prevented from operating.

### Blockage Detection in Disinfection Mode

Referring to FIG. 26, in one embodiment, when operating in disinfection mode, the apparatus 10 may also be configured to execute or implement a blockage detection algorithm 1500. The blockage detection algorithm 1500 may operate by itself or in parallel with the leak detection algorithm 1000.

The blockage detection algorithm 1500 is configured to detect and trigger an alarm is problematic blockages are detected in the flow path during disinfection mode operation. For example, during disinfection mode, the filter 1104 or disinfection tube 1100 can become completely or partially blocked due to condensate or particulate matter or due to kinking of the disinfection tube.

In this example embodiment, the blockage detection algorithm 1500 is configured at blockage evaluation stage 1502 to compare the incoming data representing the gauge pressure variable P_{gauge} to a blockage threshold P_{block}. If the pressure variable P_{gauge} is greater than the blockage threshold P_{block} for a predetermined evaluation time period, then the blockage detection algorithm triggers one or more alarm or fault actions, as shown at 1504. In this example, the predetermined evaluation time period is 5 seconds, but any other suitable time period may be used. If no blockage is detected, the blockage detection algorithm remains in the evaluation stage 1502 and continues to evaluate the incoming gauge pressure variable data P_{gauge} against the blockage threshold P_{block} to identify any blockage events or conditions.

If a blockage is detected in evaluation stage 1502, the blockage detection algorithm 1500 initiates one or more alarm actions. In this example embodiment, the blockage detection algorithm raises a blockage alarm 1504. The blockage alarm 1504 may be audible, visual (e.g. notification or message on display, and/or tactile). The blockage alarm then needs to be resolved by the user (e.g. by cleaning the disinfection tube or filter or un-kinking the tube or otherwise resolving the cause of the blockage). Once the blockage is cleared, the triggered blockage alarm or fault is resolved or cleared by the user manually restarting the disinfection mode or cycle, as shown at 1506. Once the disinfection mode is restarted, the blockage detection algorithm 1500 reverts to the evaluation stage 1502, and continues to check for any new blockage conditions as the disinfection cycle operates.

In this example, the blockage threshold P_{block} may be derived from a blockage threshold or function that is at least partly dependent on the flow rate of the flow of gases. By way of example, in this embodiment the blockage threshold may be in the form of the following:

$${P}_{block} = \frac{1}{β} ( {k}_{1} {Q}_{stpd , filt}^{2} + {k}_{2} {Q}_{stpd , filt} + {k}_{3} ) \qquad ( 3 )$$

Where Q_{stpd,filt} is the filtered STPD flow rate, k₁-k₃ are constants that are specific to the apparatus and/or flow path characteristics and/or the disinfection mode, and the value β is a function based at least partly on the sensed ambient pressure the apparatus is operating in and the sensed temperature of the flow of gases (e.g. the temperature of the flow of gases at or near the outlet of the elbow conduit or some other suitable location in the flow path). In this example, equation (3) may be quadratic functions.

The blockage detection algorithm described above may also be used in other modes of the respiratory apparatus, including therapy mode and/or drying mode, to detect blockages in the flow path. The blockage threshold P_{block} function or curve that define the blockages may be different and depend on the flow path characteristics and configuration specific to those other modes. By way of example, if the apparatus is operating in therapy mode, the blockage detection algorithm may check and monitor for a blockage based on whether the pressure variable P_{gauge} is above the blockage threshold P_{block} for a predetermined time period (e.g. 5 seconds or 10 seconds or some other evaluation time period), where the P_{block} threshold is extracted from the P_{block} threshold function or curve based at least partly on the current operating flow rate. If a blockage is detected, a blockage alarm is generated as above. The user can then resolve the blockage in the flow path, and restart the therapy mode.

### 3.4 Detailed Third Example of a Leak Detection Process—Leak Flow Rate Thresholds

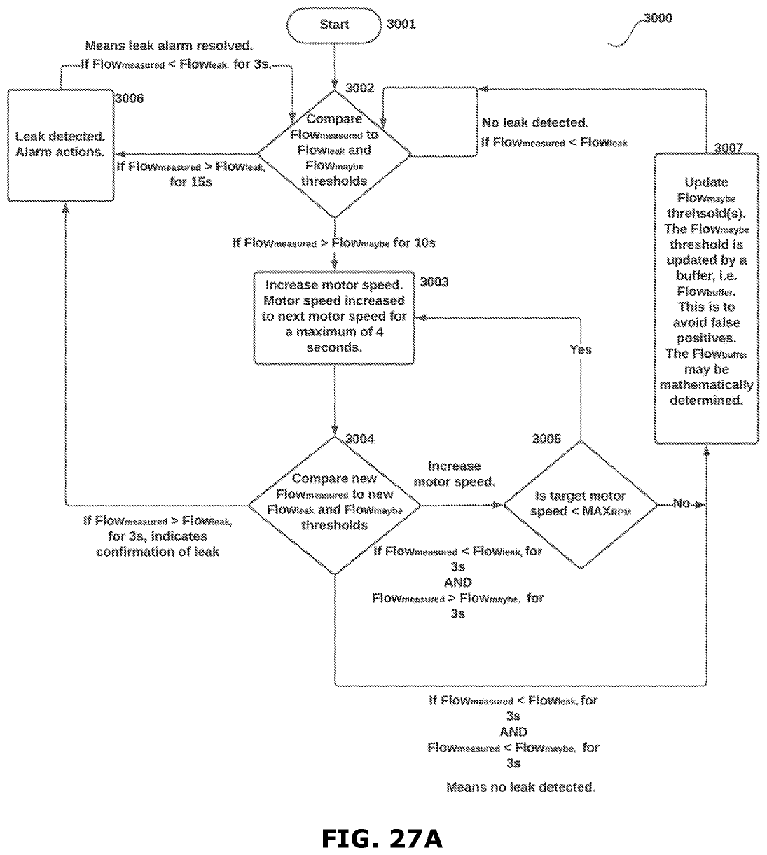

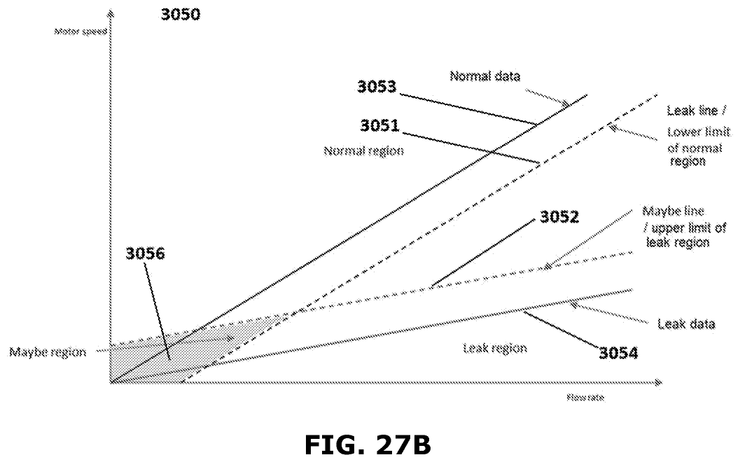

Referring to FIGS. 27A and 27B, an alternative form of two-stage leak detection algorithm 3000 will be described. The leak detection algorithm 3000 is similar to the previously described leak detection algorithms 700, 800, and 1000, except that the leak evaluation stages and decisions are based on comparing measured sensed flow rates or flow rate variable to leak thresholds, rather than comparing a sensed pressure variable to leak thresholds.

It will be appreciated that the leak detection algorithm 3000 may be adapted and configured for detecting leaks in therapy mode and non-therapy modes (e.g. drying mode and disinfection mode) in a similar manner to the previously described algorithms. In particular, the particular leak thresholds used in the evaluations may be customised to the particular type of leak being detection and/or mode of operation of the apparatus 10.

Referring to FIG. 27A, a detailed flow diagram of an embodiment of the leak detection process or algorithm 3000 is shown, by way of example only. This example algorithm 3000 utilises the similar general principles of operation of the earlier algorithms 700, 800, and 1000 previously described and it will be appreciated that the features described in regard to those algorithms may also apply to this algorithm 3000.

This embodiment of the leak detection algorithm 3000 is configured to detect any of the types of leaks in the flow path previously described in regard to the therapy mode and drying mode (e.g. leak condition caused by chamber off), and disinfection mode (e.g. filter off or disconnection of disinfection tube). The leak thresholds employed in the algorithm may be based on the type of leak being detected and/or mode of operation of the apparatus.

In this embodiment, the leak detection algorithm 3000 commences 3001 during normal operation of the apparatus (e.g. whether in therapy mode, drying mode, or disinfection mode). During normal operation, the glow generator of the apparatus generates of flow of gases at a configurable user set flow rate for a therapy session in therapy mode, or a specific pre-set or preconfigured flow rate specific to a non-therapy mode such as drying mode or disinfection mode, as previously described.

In this embodiment, the algorithm 3000 receives a flow rate variable representing or indicative of the flow rate signal or data sensed by a flow rate sensor or sensors that are configured to sense the flow rate of the flow of gases in the flow path. In some configurations, the flow rate variable may be the raw sensed flow rate signal or data. In other configurations, the flow rate variable may be a processed or filtered version of the raw sensed flow rate signal or data. In one example configuration, the flow rate variable may be the filtered STPD flow rate.

In this embodiment, the algorithm 3000 primarily has a first-stage leak evaluation 3002, and a second-stage leak evaluation which comprises the steps and evaluations or determinations at 3003, 3004, and 3005. The first-stage leak evaluation 3002 is able to determine if there is a definite leak, possible leak, or no leak. The second-stage leak evaluation is configured to resolve a possible leak determination from the first-stage as either a definite leak or no leak.

In one embodiment, the algorithm 3000 is suspended or delayed from operating until a predetermined delay period has expired from one or more particular events. These events may include any one or more of the following (depending on mode of operation): commencement of a therapy session, commencement of drying mode, commencement of disinfection cycle, commencement of normal operation or flow therapy control, resolution of a leak alarm or other alarms. In such configurations, for example, the algorithm 3000 does not operate and cannot generate or trigger a leak alarm during the delay period after one or more selected or configured such events. In other embodiments, the algorithm 3000 may be operable continuously and immediately at the start of a therapy session, drying session, disinfection cycle, or when normal flow control resumes after a leak alarm has been resolved.

### First-Stage Leak Evaluation

In this embodiment, after any required delay period has expired, the algorithm 3000 starts 3001 by proceeding to the first-stage leak evaluation at step 3002. In this first-stage leak evaluation 3002, the flow rate variable is compared to definite and possible leak thresholds to determine the leak state of the apparatus, e.g. leak detected, possible leak, or no leak. In this embodiment, one or each condition may be considered as being satisfied or detected based on respective evaluation criteria being satisfied, as will be explained below. In this embodiment, the various evaluations based on comparing the flow rate to the one or more threshold(s), may occur concurrently or in parallel. In other configurations, the evaluations of which leak condition is satisfied may be configured to occur sequentially, or in a particular conditional order.

While operating in the first-stage leak evaluation 3002, the apparatus can be considered as being in normal operation with no leak detected, i.e. in the no leak state. In this state or stage, the respiratory apparatus continues with normal operation and normal flow control in accordance with the set flow rate (depending on the mode of operation, e.g. therapy mode, drying mode, or disinfection mode).

In this embodiment, the algorithm retrieves and/or receives input data representing the measured or sensed flow rate of the flow of gases and the leak thresholds, for evaluation. The input data may be retrieved in accordance with a particular sampling frequency or continuously as the data becomes available from the sensors and/or main controller and/or memory of the apparatus. In one configuration, the input data may be a moving average based on a moving window of data. The window of data may be determined based on a configuration time period or number of data samples, for example. In one example, the input data may be a 10 second moving average of the sensed flow rate and leak thresholds. In another configuration, the input data used by the leak detection algorithm may be the latest instantaneous data for the sensed flow rate and leak thresholds.

In this embodiment, at step 3002, one of the evaluations undertaken by the algorithm is to determine a definite leak condition. In this embodiment, to determine a definite leak condition, the algorithm compares the flow rate variable Flow_{measured} to a definite leak threshold Flowₗₑₐₖ (which is a flow rate threshold in this embodiment) representing a definite leak condition. If the flow rate variable Flow_{measured} is more than the definite leak threshold Flowₗₑₐₖ for a minimum evaluation time, then a leak condition is satisfied and the algorithm raises or generates a leak alarm at 3006. In one configuration, the flow rate variable Flow_{gauge} must be above the definite leak threshold Flowₗₑₐₖ for a minimum evaluation time period of 15 seconds, although it will be appreciated this time period may be adjusted or varied in other embodiments.

If the leak detection condition is satisfied based on the evaluation at step 3002, the algorithm moves to the leak alarm or leak detected state or stage at 3006. In the leak detected state 3006, the algorithm may trigger one or more alarm actions such as, but not limited to, generating an audible, visual or tactile leak detection alarm, and/or controlling or halting the flow rate or motor speed, controlling other apparatus components, and/or other control actions, as previously explained in regard to algorithms 700, 800, and 1000. Any of the alarm actions of the previously described algorithms 700, 800, and 1000 may be employed, depending on the type of leak being detected and/or mode of operation of the apparatus.

Once in the leak detected state 3006, the algorithm 3000 may also be configured to determine or evaluate whether the leak alarm has been resolved, for example whether the chamber has been reconnected or installed back into the apparatus and/or flow path in therapy mode or drying mode, or whether the filter has been reconnected or installed in disinfection mode. In this embodiment, the algorithm at 3006 continually evaluates or determines whether the leak has been resolved by comparing the flow rate variable Flow_{measured} to the definite leak threshold Flowₗₑₐₖ or another alternative threshold specific to confirm leak resolution. In this example embodiment, if the flow rate variable Flow_{measured} is below the Flowₗₑₐₖ threshold for a minimum evaluation time period, which in this embodiment is 3 seconds but this could be adjusted in other embodiments, the leak alarm is considered as resolved. If the leak alarm is resolved, the algorithm returns to the first-stage leak evaluation 3002 and normal operation or normal flow control in accordance with the set flow rate of the applicable mode (therapy mode, drying mode, or disinfection mode). If the flow rate variable does drop below the Flowₗₑₐₖ threshold for more than the minimum evaluation time period, then the algorithm remains in the leak detected state 3006 and continues to check for resolution of the leak alarm.

In this embodiment, at step 3002, one of the other evaluations undertaken by the algorithm is to determine a possible leak condition. In this embodiment, to determine a possible leak condition, the algorithm compares the flow rate variable Flow_{measured} to a possible leak threshold Flow_{maybe} (which is a flow rate threshold in this embodiment) representing a possible or potential leak condition. In this embodiment, if the flow rate variable Flow_{measured} is above the possible leak threshold Flow_{maybe} for a minimum evaluation time period, a possible leak condition is satisfied and the algorithm moves to the second-stage leak evaluation (which commences at 1003) to resolve the identified possible leak as a definite leak or no leak. In this embodiment, the minimum evaluation time period is 10 seconds, but this may be varied in different embodiments.

If neither of the definite leak or possible leak conditions are met based on the evaluations described above, the algorithm considers no leak is detected, and remains in the first-stage leak evaluation 3002 in normal operation, i.e. no leak condition remains satisfied, and the apparatus continues with flow control at normal operation for the mode of operation applicable (e.g. therapy mode, drying mode, disinfection mode).

### Second-Stage Leak Evaluation

If the algorithm detects a possible leak condition at 3002, the algorithm moves to the second-stage leak evaluation, commencing at step 3003. In this embodiment, before entering the second-stage leak evaluation to resolve the possible leak as a definite leak or no leak, the algorithm 3000 is configured to store the measured flow rate (e.g. sensed by a flow rate sensor in the flow path or other sensing configuration) and the motor speed before entering the second-stage leak evaluation at step 3003. In one configuration, the stored values of the measured flow rate and/or motor speed may represent or be extracted from moving averages of this measured or sensed variable, as explained above. For example, in one configuration, the algorithm either receives during operation or calculates during operation a moving average of the measured flow rate variable and/or motor speed, from which it can extract and store values prior to entering the second-stage leak evaluation. By way of example, the stored values of measured flow rate or flow rate variable and/or motor speeds may be used in later algorithm steps involving updating and/or adjusting one or more of the leak threshold limits.

In this embodiment, the second-stage leak evaluation comprises increasing the current target motor speed by a predetermined increment or to a first or next pre-set higher motor speed at 3003. In this embodiment, the algorithm is configured to increase and lock the motor speed at the first higher motor speed for a predetermined or maximum time period. In one configuration, the time period may be 4 seconds, but this may be varied in alternative embodiments. As will be explained below, during the or each iteration to a next higher motor speed, the higher motor speed is maintained for the configured time period (e.g. 4 seconds in this example) while the second-stage leak evaluations 3004 are undertaken to attempt to resolve the possible leak as a definite leak condition or a no leak condition.

Once at the higher motor speed after the increase at 3003, the algorithm 3000 commences with one or more second-stage leak evaluations at 3004. For example, the updated or new flow rate variable Flow_{measured} at the higher motor speed is compared to new or updated definite Flowₗₑₐₖ and possible Flow_{maybe} thresholds to determine whether the possible leak condition can be resolved as a definite leak condition or a no leak condition. Each condition may again have its own evaluation criteria to satisfy, based on comparing the flow rate variable Flow_{measured} to one or more of the leak thresholds, as will be described below.

In this embodiment, at step 3004, one of the evaluations undertaken by the algorithm is to resolve the possible leak condition as a definite leak condition. In this embodiment, to determine a possible leak condition, the algorithm compares the updated flow rate variable Flow_{measured} to an updated definite leak threshold Flowₗₑₐₖ. In this embodiment, the new Flowₗₑₐₖ threshold is updated or determined based at least partly on the new higher motor speed of the flow generator. If the flow rate variable Flow_{measured} is above the Flowₗₑₐₖ threshold for a minimum evaluation time period (e.g. 3 seconds or another configurable time period), the possible leak condition is confirmed or resolved as a definite leak condition, and the algorithm progresses to the leak detected state 3006 and generates a leak alarm or initiates one or more alarm actions as described previously.

In this embodiment, one of the other evaluations undertaken by the algorithm is to resolve the possible leak condition as a no leak condition. In this embodiment, to determine a no leak condition, the algorithm compares the updated flow rate variable Flow_{measured} to updated possible leak threshold Flow_{maybe} and updated definite leak threshold Flowₗₑₐₖ. As explained above, the updated thresholds may be extracted or determined based at least partly on the new higher motor speed of the flow generator. If the updated flow rate variable Flow_{measured} is below the Flow_{maybe} threshold and below the Pₗₑₐₖ threshold for a minimum evaluation time (e.g. 3 seconds or another configurable time period), then the possible leak is resolved as no leak detected, and the algorithm reverts to the first-stage evaluation 3002 and normal operation and/or flow control for the current mode of operation.

In this embodiment, upon exiting the second-stage leak evaluation at 3004 (after resolving the possible leak is not a leak) and prior to reverting to step 3002 and normal operation 3001, the algorithm can optionally execute a threshold adjustment process at 3007. In this embodiment, the threshold adjustment process 3007 can be configured to adjust or reduce the Flow_{maybe} threshold to prevent the algorithm endlessly looping in the same scenario in future checks. In one configuration, the Flow_{maybe} threshold may be adjusted based on an adjustment or correction function 3007 having one or more variables.

By way of example, the variables of the adjustment or correction function may comprise the stored flow rate variable and/or motor speed stored prior to entering the second-stage leak evaluation at step 3003, as previously discussed. Additionally, the adjustment or correction function may include one or more further variables or constants. In one configuration, the adjustment or correction function may have a constant variable that is dependent on the stored flow rate variable, such that the magnitude of adjustment may vary depending on the stored flow rate variable. In one configuration, the constant variable may be configured to cause the adjustment or correction function to make smaller adjustments to the Flow_{maybe} threshold at lower flow rates or lower flow rate ranges.

In one embodiment, the threshold adjustment function 3007 may comprise updating the Flow_{maybe} threshold based at least partly on a buffer value Flow_{buffer}. In one configuration, the Flow_{buffer} value may be at least partly dependent on the flow rate variable and/or motor speed stored after exiting the first-stage 3002. In another configuration, the Flow_{buffer} value may be a constant. In one configuration, the Flow_{maybe} threshold is extracted from a threshold function that is dependent on the motor speed and one or more constants. In this example configuration, the threshold adjustment function is configured to adjust the one or more constants of the threshold function based at least partly on the Flow_{buffer} value. In another example, the threshold adjustment function is configured to adjust the one or more constants of the threshold function based at least partly on the Flow_{buffer} value, and the flow rate variable and motor speed value stored after existing the first-stage 3002.

In some configurations of the adjustment or correction function 3007, the Flow_{maybe} threshold at the discrete motor speed sensed prior to entering the second-stage leak evaluation at 3003 may be increased by a calculated or predetermined amount. In other configurations, the entire Flow_{maybe} threshold function or threshold curve may be increased by a calculated or predetermined amount across the entire operating flow rate or motor speed range.

Reverting to the second-stage leak evaluations at 3004, if neither of the definite leak or no leak conditions are satisfied based on the evaluations described above, the possible leak condition is considered as unresolved. In this embodiment, if the possible leak condition is unresolved for a predetermined time period based on a timer (e.g. 4 seconds in this example, but could be another configurable time period), then the second-stage leak evaluation process repeats, but at a further higher motor speed.

In this embodiment, if the timer expires and the possible leak condition is unresolved, the algorithm exits 3004 and initiates a motor speed check 3005. In this embodiment, the motor speed check 3005 comprises comparing the current target motor speed against a pre-set or configurable motor speed threshold (MAX_{RPM}), before repeating or looping back to the start 3003 of the second-stage leak evaluation.

In this embodiment, if the current target motor speed is equal to or above the motor speed threshold MAX_{RPM} at the motor speed check 3005, the algorithm exits the second-stage leak evaluation and resolves the possible leak as a no leak condition, and reverts to the first-stage 3002 and normal operation via the threshold adjustment process 3007 previously explained. The motor speed check 3005 provides an additional exit condition for the algorithm, and prevents the algorithm from endlessly looping in the second-stage and/or from increasing the motor speed beyond safety or component thresholds.

If the current target motor speed is below the motor speed threshold MAX_{RPM} at the motor speed check 3005, the algorithm loops back to step 3003, and again increases the first higher motor speed to a second or next higher motor speed, and repeats the evaluations at 3004 again, in an attempt to resolve the possible leak as a definite leak or no leak condition.

As shown, the second-stage comprises continuously or repeatedly incrementing the motor speed by a predetermined amount or to a next predetermined higher motor speed, and then undertaking evaluations at 3004 until the leak condition is satisfied, the no leak condition is satisfied, or the motor speed check fails at 3005. As will be appreciated, depending on the evaluations, the second-stage of the algorithm may complete once, twice or multiple times, prior to exiting back to 3002 (no leak) or 3006 (leak detected).

At step 3003, it will be appreciated that the increments in motor speed may be pre-set or configurable increments or may increment to the next highest motor speed in a pre-set or configurable series or array of preselected discrete higher motor speeds. By way of example only, in one configuration, the motor speed increase at step 806 is based on incrementing the current motor speed to the next highest motor speed in the series or array comprising motor speeds of 5000 rpm, 6750 rpm, 8500 rpm, 10250 rpm, and 12000 rpm, such that the motor speed increases by 1750 rpm each cycle or loop of the second-stage leak evaluation. In this example, there are five possible discrete motor speed stages in which the flow rate variable is compared against the Flow_{maybe} and Flowₗₑₐₖ thresholds, but this may be varied to more or less stages as desired.

In this embodiment, the leak detection algorithm 3000 repeats once it returns to the first-stage leak evaluation and normal operation at 3002. In one configuration, the leak detection algorithm continually operates as it receives updated real-time flow rate variable data from the controller and/or flow rate sensor(s). In other configurations, the leak detection algorithm may be configured to operate periodically. As previously discussed, in some configurations, upon the apparatus commencing or re-commencing normal operation, the leak evaluation comparisons against the leak thresholds may be suspended or delayed for a predetermined delay period after specific events. Such events may include, but are not limited to, commencement of a new therapy session or new user set flow rate, commencement of a drying mode session, commencement of a disinfection cycle, resolution of a prior leak alarm at 3006, or otherwise when normal operation or flow control is resumed at 3002, for example after a leak alarm has been resolved or after exiting the second-stage leak evaluation with no leak detected.

### Leak Thresholds

An example of the leak thresholds associated with the leak detection algorithm 3000 will now be further explained. As discussed, the definite leak threshold Flowₗₑₐₖ and possible leak threshold Flow_{maybe} for each leak evaluation comparison may be based on the motor speed operating at the time of the evaluation comparison. In one configuration, the Flowₗₑₐₖ and Flow_{maybe}, thresholds may be dependent on or a function of at least sensed motor speed of the flow generator, and optionally one or more other variables or operating characteristics of the apparatus and/or flow of gases. The Flowₗₑₐₖ and Flow_{maybe} thresholds may be functions, threshold curves or lines, or provided in the form of a look-up table. The thresholds may be extracted from the function, threshold curves or lines, or look-up tables based on at least the current sensed motor speed of the flow generator, and any other optional variables, at each stage of the leak detection algorithm.

Referring to FIG. 27B, a motor speed vs flow rate plot 3050 is shown, to demonstrate the nature of the leak thresholds used in the leak detection algorithm 3000. As previously explained, the leak thresholds are derived based on the motor speed versus flow rate characteristic associated with the flow of gases in the apparatus operating normally with no leak (e.g. chamber on in therapy or drying modes, or filter on in disinfection mode), and the apparatus operating with a leak (e.g. with the chamber off in therapy or drying modes, or filter off in disinfection mode).

An example Flowₗₑₐₖ threshold function is depicted as a threshold curve or line 3051 on motor speed vs flow rate graph at 3050. An example Flow_{maybe} threshold function is depicted as a threshold curve or line 3052 on motor speed vs flow rate graph at 3050. As shown, in this embodiment, the motor speed and flow rate characteristic has a linear (e.g. straight-line) relationship. The thresholds lines 3051 and 3052 will vary depending on the mode of operation e.g. therapy mode and drying mode may have similar threshold lines, while the disinfection mode will have a different threshold line.

The function or curve or line or data represented by 3053 is the sensed motor speed of the flow generator against the operational flow rate range, for a normally operating apparatus with no leak (although this line or characteristic will depend on the mode of operation). The function or curve or line or data represented by 3054 is the sensed motor speed of the flow generator against the operational flow rate range, for an apparatus operating with a leak (e.g. due to the chamber being off or filter being off depending on the mode of operation). The leak data line 3054 will vary depending on mode of operation. As shown in the lower flow rate region highlighted at 3056, the motor speed vs flow rate lines 3053 and 3054 for normal and leaky operation converge in this region, thereby making it difficult to discriminate a genuine or definite leak condition (e.g. chamber off or filter off) at lower flow rates. The leak detection algorithm of this disclosure provides a means of addressing this issue, by incrementally increasing the motor speed to provide clearer discrimination between the curves for definitively determining a leak condition or a no leak condition.

The heater plate check processes and configurations and alternatives described in section 3.2 may also be applied to the algorithm 3000, in alternative embodiments.

### 4. Example Embodiments of Single-Threshold Leak Detection Process

In another embodiment, a single-threshold leak detection process or algorithm may be implemented by the controller of the apparatus. In this single-threshold leak detection process, the algorithm is configured to either compare the incoming sensed pressure variable representing the sensed pressure characteristic of the flow of gases in the flow path of the apparatus or the incoming flow rate variable to a single leak threshold. In this embodiment, the leak threshold may be a leak pressure threshold for embodiments using the incoming sensed pressure variable or a leak flow rate threshold for embodiments using the incoming flow rate variable. The single leak threshold may be represented by a threshold curve, function, equation, model or look-up table, which defines the thresholds over the entire or at least a portion of the operating flow rate range of the apparatus, similar to the leak thresholds discussed in regard to the previous embodiments. As with the previous embodiments, the leak threshold used in the evaluations to determine whether there is a possible leak condition, definite leak condition, or no leak condition, is primarily dependent on or a function of the measured flow rate and/or motor speed operating at the time of the evaluation, and optionally one or more other variables representing operating characteristics of the apparatus and/or flow of gases.

The single-threshold leak detection method or process is also a two-stage or dual-stage process, similar to the previous embodiments. In this embodiment, when the apparatus is operating normally, the leak detection algorithm operates in a first-stage leak evaluation, and checks or compares the sensed pressure variable or flow rate variable against the leak threshold associated with the current operating conditions (e.g. flow rate and/or motor speed for example). Based on the comparison, the leak detection algorithm will either determine that a no leak condition is satisfied, or that a possible leak condition is satisfied. In one embodiment, if the sensed pressure variable is above the leak pressure threshold or the flow rate variable is below the leak flow rate threshold, the algorithm considers there to be no leak (i.e. no leak condition satisfied), and continues normal operation and continues in the first-stage leak evaluation checks. If the sensed pressure variable is below the leak pressure threshold or the flow rate variable is above the leak flow rate threshold, the algorithm considers there is a possible leak, i.e. a possible leak condition is satisfied.

If a possible leak condition is satisfied from the first-stage leak evaluation, the algorithm moves to a second-stage leak evaluation to resolve the possible leak as either a no leak condition or a definite leak condition. In this second-stage leak evaluation, the algorithm is configured to increase the operating flow rate and/or motor speed of the apparatus to a higher flow rate and/or motor speed. The increase in flow rate and/or motor speed may be a predetermined increment, a dynamically determined increment, or an increment determined by a function that is dependent on one or more variables such as the current operating conditions of the apparatus (e.g. flow rate and/or motor speed). At the higher flow rate and/or motor speed, the algorithm is configured to check or compare the updated or new sensed pressure variable or new flow rate variable to the updated respective leak threshold associated with the higher flow rate and/or motor speed. If the new sensed pressure variable is higher than the new leak pressure threshold or the new flow rate variable is below the new leak flow rate threshold, the possible leak is resolved as a no leak condition, and the algorithm reverts to the first-stage leak evaluation and normal operation. If the new sensed pressure variable is lower than the new leak pressure threshold or the new flow rate variable is above the new leak flow rate threshold, the possible leak is resolved or confirmed as a definite leak condition, and the algorithm moves to a leak detected state or condition, may execute one or more alarm actions, as with the previous embodiments.

In this embodiment, the leak detection algorithm is a dual-stage approach of an initial leak assessment at the normal operating conditions, and then moves to a second confirmatory leak assessment at a higher operating condition (e.g. higher flow rate and/or motor speed) where discriminating between a leak and no leak conditions is easier or can be achieved with a higher level of confidence based on a single leak threshold function.

### 5. Terminology

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The scope of the present disclosure is not intended to be limited by the specific disclosures of embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A respiratory apparatus that is configured to provide a flow of gases to a user for respiratory therapy comprising:
a flow generator that is operable to generate a flow of gases, wherein the flow generator is motor-driven;
a flow path for the flow of gases through the respiratory apparatus to a gases outlet of the respiratory apparatus;
a humidifier that is operable to heat and humidify the flow of gases, the humidifier comprising a removable humidification chamber in the flow path;
a pressure sensor configured to generate a pressure variable representing a sensed pressure characteristic of the flow of gases in the flow path; and
a controller that is operatively connected to the flow generator and operable to control a flow rate of the flow of gases by controlling a motor speed of the flow generator, wherein the controller is configured during operation to execute a leak detection process for detecting leaks in the flow path by:
initiating a first-stage leak evaluation comprising comparing the pressure variable to a first leak threshold, the first leak threshold representing a possible leak condition; and
initiating a second-stage leak evaluation if the pressure variable is below the first leak threshold thereby indicating a possible leak, the second-stage leak evaluation comprising:
increasing the motor speed of the flow generator from a current motor speed to a higher motor speed,
comparing a new pressure variable at the higher motor speed to the first leak threshold and a second leak threshold, the second leak threshold representing a definite leak condition, and then:
generating a leak alarm and exiting the second-stage leak evaluation if the new pressure variable is below the second leak threshold thereby confirming the possible leak as a definite leak; or
exiting the second-stage leak evaluation without generating an alarm if the new pressure variable is above the first leak threshold and second leak threshold thereby resolving the possible leak as no leak; or
repeating the second-stage leak evaluation at a further higher motor speed or speeds until the possible leak is confirmed as a leak or resolved as no leak.

2. The respiratory apparatus of claim 1 wherein the first-stage leak evaluation comprises comparing the pressure variable to the first leak threshold and second leak threshold, and wherein the controller is configured to initiate the second-stage leak evaluation if the pressure variable is below the first leak threshold and above the second leak threshold thereby indicating a possible leak.

3. The respiratory apparatus of claim 1 wherein the first-stage leak evaluation comprises comparing the pressure variable to the second leak threshold, and wherein the controller is configured to generate a leak alarm if the pressure variable is below the second leak threshold.

4. (canceled)

5. The respiratory apparatus of claim 1 wherein exit conditions comprise generating a leak alarm or initiating a second-stage leak evaluation to resolve a possible leak as a definite leak or no leak.

6. The respiratory apparatus of claim 1 wherein, in the second-stage leak evaluation, increasing the motor speed of the flow generator comprises increasing the motor speed by a set increment to a first motor speed or to a second motor speed from a predetermined series or array of higher motor speeds, wherein the second motor speed is higher than the first motor speed.

7. The respiratory apparatus of claim 6 wherein, in the second-stage leak evaluation, the controller is configured to hold the flow generator at the higher motor speed for a predetermined time period while comparing the new pressure variable to the first leak threshold and/or the second leak threshold to resolve the possible leak as a definite leak or no leak.

8. The respiratory apparatus of claim wherein the controller is configured to repeat the second-stage leak evaluation at a second higher motor speed if the possible leak is not confirmed as a leak or resolved as no leak at the second higher motor speed within a predetermined time period.

9. The respiratory apparatus of claim 1 wherein the controller is configured to exit the second-stage leak evaluation depending on a comparison of the current motor speed to a motor speed threshold.

10. The respiratory apparatus of claim 9 wherein the controller is configured to exit the second-stage leak evaluation if the current motor speed operating during the second-stage leak evaluation not below a motor speed threshold.

11. The respiratory apparatus of claim 1 wherein the controller is configured to adjust the first leak threshold if the second-stage leak evaluation is exited without generating an alarm.

12. The respiratory apparatus of claim 11 wherein the controller is configured to adjust the first leak threshold by reducing the first leak threshold associated with the flow rate and/or motor speed operating during the first-stage leak evaluation.

13. The respiratory apparatus of claim 1 wherein values of the first leak threshold and the second leak threshold are at least partly dependent on or a function of the flow rate and/or motor speed operating during comparison.

14. The respiratory apparatus of claim 1 wherein the first leak threshold and the second leak threshold are extracted from respective pressure-flow characteristic curves and/or representative look-up tables designating the first leak threshold and the second leak threshold for a range of flow rates and/or motor speeds.

15. (canceled)

16. (canceled)

17. (canceled)

18. The respiratory apparatus of claim 1 wherein the controller is configured to initiate one or more alarm actions when generating a leak alarm.

19. The respiratory apparatus of claim 18 wherein the one or more alarm actions comprise freezing the flow rate and/or motor speed of the flow generator to current operating settings.

20. The respiratory apparatus of claim 18 wherein the one or more alarm actions comprise generating a notification or indication of the leak on a display of the respiratory apparatus.

21. (canceled)

22. (canceled)

23. The respiratory apparatus of claim 1 wherein the controller is operable to detect one or more different types of leaks in the flow path of the respiratory apparatus, each different type of leak having its own respective first and second leak thresholds.

24. (canceled)

25. The respiratory apparatus of claim 1 wherein, after an alarm is generated, the controller is configured to disable the alarm and revert to normal operation if the pressure variable rises above the second leak threshold or an alternative resolution leak threshold for a minimum time period.

26. (canceled)

27. (canceled)

28. The respiratory apparatus of claim 1 wherein the humidifier of the respiratory apparatus further comprises a heater plate that is operable to heat the removable humidification chamber, and wherein the controller is further configured to execute a heater plate check process to further confirm or validate a definite leak identified in the second-stage leak evaluation before generating the leak alarm.

29. The respiratory apparatus of claim 28 wherein the heater plate check process comprises applying a power or temperature process to the heater plate and evaluating heating rate or and/or cooling rate of the heater plate against one or more thresholds based on a temperature sensor of or associated with the heater plate to thereby determine presence or absence of a humidification chamber being in thermal contact with the heater plate, the absence of the humidification chamber validating a definite leak condition.

30. (canceled)

31. (canceled)

32. (canceled)

33. (canceled)

34. (canceled)

35. (canceled)
